SEQUENCE LISTING (119 sequences)

SEQ ID NO: 1 - protein, 329 aa, Homo sapiens
ASTKGPSVFPLAPSSKSTSGGTAALGCLVEDYFPEPVTVSWNSGALTSGVHTFPAVLQSSGLYSLSSVVTVPSSSLGTQTYICNVNHKPSNTKVDEKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 3 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 4 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 6 - protein, 226 aa, synthetic construct
  REGION 1..226: note = human IgG1 Fc-region derived Fc-region polypeptide with the mutations L234A, L235A
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 7 - protein, 226 aa, synthetic construct
  REGION 1..226: note = human IgG1 Fc-region derived Fc-region polypeptide with Y349C, T366S, L368A and Y407V mutations
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 8 - protein, 226 aa, synthetic construct
  REGION 1..226: note = human IgG1 Fc-region derived Fc-region polypeptide with S354C, T366W mutations
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - protein, 226 aa, synthetic construct
  REGION 1..226: note = human IgG1 Fc-region derived Fc-region polypeptide with L234A, L235A mutations and Y349C, T366S, L368A, Y407V mutations
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 10 - protein, 226 aa, synthetic construct
  REGION 1..226: note = human IgG1 Fc-region derived Fc-region polypeptide with a L234A, L235A and S354C, T366W mutations
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 11 - protein, 226 aa, synthetic construct
  REGION 1..226: note = human IgG1 Fc-region derived Fc-region polypeptide with a P329G mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 226 aa, synthetic construct
  REGION 1..226: note = human IgG1 Fc-region derived Fc-region polypeptide with L234A, L235A mutations and P329G mutation
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 13 - protein, 226 aa, synthetic construct
  REGION 1..226: note = human IgG1 Fc-region derived Fc-region polypeptide with a P329G mutation and Y349C, T366S, L368A, Y407V mutations
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 14 - protein, 226 aa, synthetic construct
  REGION 1..226: note = human IgG1 Fc-region derived Fc-region polypeptide with a P329G mutation and S354C, T366W mutation
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 226 aa, synthetic construct
  REGION 1..226: note = human IgG1 Fc-region derived Fc-region polypeptide with L234A, L235A, P329G and Y349C, T366S, L368A, Y407V mutations
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 16 - protein, 226 aa, synthetic construct
  REGION 1..226: note = human IgG1 Fc-region derived Fc-region polypeptide with L234A, L235A, P329G mutations and S354C, T366W mutations
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 229 aa, synthetic construct
  REGION 1..229: note = human IgG4 Fc-region derived Fc-region polypeptide with S228P and L235E mutations
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 229 aa, synthetic construct
  REGION 1..229: note = human IgG4 Fc-region derived Fc-region polypeptide with S228P, L235E mutations and P329G mutation
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 229 aa, synthetic construct
  REGION 1..229: note = human IgG4 Fc-region derived Fc-region polypeptide with S354C, T366W mutations
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 229 aa, synthetic construct
  REGION 1..229: note = human IgG4 Fc-region derived Fc-region polypeptide with Y349C, T366S, L368A, Y407V mutations
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 22 - protein, 229 aa, synthetic construct
  REGION 1..229: note = human IgG4 Fc-region derived Fc-region polypeptide with a S228P, L235E and S354C, T366W mutations
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 229 aa, synthetic construct
  REGION 1..229: note = human IgG4 Fc-region derived Fc-region polypeptide with a S228P, L235E and Y349C, T366S, L368A, Y407V mutations
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 24 - protein, 229 aa, synthetic construct
  REGION 1..229: note = human IgG4 Fc-region derived Fc-region polypeptide with a P329G mutation
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 229 aa, synthetic construct
  REGION 1..229: note = human IgG4 Fc-region derived Fc-region polypeptide with a P239G and Y349C, T366S, L368A, Y407V mutations
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 26 - protein, 229 aa, synthetic construct
  REGION 1..229: note = human IgG4 Fc-region derived Fc-region polypeptide with a P329G and S354C, T366W mutations
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 229 aa, synthetic construct
  REGION 1..229: note = human IgG4 Fc-region derived Fc-region polypeptide with a S228P, L235E, P329G and Y349C, T366S, L368A, Y407V mutations
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVCTLPPSQEEMTKNQVSLSCAVKGFYPSDIAVEWESNGQPENNYKTTPPVLDSDGSFFLVSRLTVDKSRWQEGNVFSCSVMHEALHNHYTQKSLSLSLGK

SEQ ID NO: 28 - protein, 229 aa, synthetic construct
  REGION 1..229: note = human IgG4 Fc-region derived Fc-region polypeptide with a S228P, L235E, P329G and S354C, T366W mutations
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPCQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 107 aa, Homo sapiens [sequence omitted]

SEQ ID NO: 30 - protein, 106 aa, Homo sapiens
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 31 - protein, 20 aa, synthetic construct
  REGION 1..20: note = peptidic linker
GGGGSGGGGSGGGGSGGGGS

SEQ ID NO: 32 - protein, 32 aa, synthetic construct
  REGION 1..32: note = peptidic linker
GGGGSGGGGSGGGGSGGGGSGGGGSGGGGSGG

SEQ ID NO: 33 - DNA, 34 bp, synthetic construct
  misc_feature 1..34: note = L3
  source 1..34: mol_type = other DNA
ataacttcgtataaagtctcctatacgaagttat

SEQ ID NO: 34 - DNA, 34 bp, synthetic construct
  misc_feature 1..34: note = 2L
  source 1..34: mol_type = other DNA
ataacttcgtatagcatacattatacgaagttat

SEQ ID NO: 35 - DNA, 34 bp, synthetic construct
  misc_feature 1..34: note = loxFas
  source 1..34: mol_type = other DNA
acaacttcgtatatacctttctatacgaagttgt

SEQ ID NO: 36 - DNA, 129 bp, Simian virus 40
  source 1..129: mol_type = other DNA
aacttgtttattgcagcttataatggttacaaataaagcaatagcatcacaaatttcacaaataaagcatttttttcaccattctagttgtggtttgtccaaactcatcaatgtatcttatcatgtctg

SEQ ID NO: 37 - DNA, 225 bp, Bos taurus
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 38 - DNA, 73 bp, Homo sapiens
  source 1..73: mol_type = other DNA
caggataatatatggtagggttcatagccagagtaacctttttttttaatttttattttattttatttttgag

SEQ ID NO: 39 - DNA, 288 bp, Simian virus 40
  source 1..288: mol_type = other DNA
agtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcg

SEQ ID NO: 40 - protein, 199 aa, Streptomyces alboniger
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

SEQ ID NO: 41 - DNA, 906 bp, Streptomyces alboniger
  source 1..906: mol_type = other DNA
gtcgacatcgcggccgaaccggtcgtgagcccctgggacatcgccgccctccagatcctcgtggaggaggcgggcggcgtctgcaccgacctgctcggcggctcgccccagcgcgggacgggtgccctgtccgccaacccggagctgcaccggctcgccgtggaggccctcgccgcccccgcggccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgg

SEQ ID NO: 42 - DNA, 608 bp, synthetic construct
  source 1..608: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacgtcagatc

SEQ ID NO: 43 - DNA, 696 bp, synthetic construct
  source 1..696: mol_type = other DNA
gttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattagcatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctccgtttagtgaacgtcagatctagctctgggagaggagcccagcactagaagtcggcggtgtttccattcggtgatcagcactgaacacagaggaagcttgccgccacc

SEQ ID NO: 44 - DNA, 2125 bp, Human cytomegalovirus
  source 1..2125: mol_type = genomic DNA
ctgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacggtttaaacgccgccacc

SEQ ID NO: 45 - protein, 42 aa, Homo sapiens [sequence omitted]

SEQ ID NO: 46 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 47 - protein, 297 aa, Homo sapiens
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 48 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 49 - protein, 230 aa, Homo sapiens
MEPLRLLILLFVTELSGAHNTTVFQGVAGQSLQVSCPYDSMKHWGRRKAWCRQLGEKGPCQRVVSTHNLWLLSFLRRWNGSTAITDDTLGGTLTITLRNLQPHDAGLYQCQSLHGSEADTLRKVLVEVLADPLDHRDAGDLWFPGESESFEDAHVEHSISRSLLEGEIPFPPTSILLLLACIFLIKILAASALWAAAWHGQKPGTHPPSELDCGHDPGYQLQTLPGLRDT

SEQ ID NO: 50 - protein, 441 aa, synthetic construct
  REGION 1..441: note = anti-pTau antibody HC
EVQVLESGGGLVQPGGSLRLSCAVSGFSLSSNAINWVRQAPGKGLEWVGYIAVSGNTYYASWAKGRFTISRDNSKNTVYLQMNSLRAEDTAVYYCGKSNIWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 51 - protein, 218 aa, synthetic construct
  REGION 1..218: note = anti-pTau antibody LC
AQVMTQSPSSLSASVGDRVTITCRSSQSVRTNRLAWFQQKPGQPPKRLIYSASTLDYGVPSRFSGSGSGTEFTLTISSLQPEDFATYYCLGYFDSSADIVAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 449 aa, synthetic construct
  REGION 1..449: note = anti-Her3 antibody HC
QVQLVQSGAEVKKPGASVKVSCKASGYTFRSSYISWVRQAPGQGLEWMGWIYAGTGSPSYNQKLQGRVTMTTDTSTSTAYMELRSLRSDDTAVYYCARHRDYYSNSLTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 220 aa, synthetic construct
  REGION 1..220: note = anti-Her3 antibody LC
DIVMTQSPDSLAVSLGERATINCKSSQSVLNSGNQKNYLTWYQQKPGQPPKLLIYWASTRESGVPDRFSGSGSGTDFTLTISSLQAEDVAVYYCQSDYSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 119 aa, synthetic construct
  REGION 1..119: note = anti-CD20 antibody VH
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 55 - protein, 112 aa, synthetic construct
  REGION 1..112: note = anti-CD20 antibody VL
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 56 - protein, 116 aa, synthetic construct
  REGION 1..116: note = humanized anti-alpha synuclein acntibody 9E4 VH
EVQLVESGGGLVQPGGSLRLSCAASGFTFSNYGMSWVRQAPGKGLEWVASISSGGGSTYYPDNVKGRFTISRDDAKNSLYLQMNSLRAEDTAVYYCARGGAGIDYWGQGTLVTVSS

SEQ ID NO: 57 - protein, 113 aa, synthetic construct
  REGION 1..113: note = humanized anti-alpha synuclein acntibody 9E4 VL
DIQMTQSPSSLSASVGDRVTITCKSIQTLLYSSNQKNYLAWFQQKPGKAPKLLIYWASIRKSGVPSRFSGSGSGTDFTLTISSLQPEDLATYYCQQYYSYPLTFGGGTKLEIK

SEQ ID NO: 58 - protein, 115 aa, Mus musculus
QVQLQQSGAELAKPGTSVQMSCKASGYTFTNYWMNWIKARPGQGLEWIGATNPNNGYTDYNQRFKDKAILTADKSSNTAYMHLSSLTSEDSAVYFCASGGHLAYWGQGTVVTVSA

SEQ ID NO: 59 - protein, 112 aa, Mus musculus
DVVMTQIPLYLSVSPGDQASISCRSSQSLFHSKGNTYLHWYLQKPGQSPKLLINRVSNRFSGVPDRFSGSGSGTDFTLKISGVEAEDLGVYFCSQSAHVPWTFGGGTKLEIR

SEQ ID NO: 60 - protein, 115 aa, Mus musculus
VQLQQSGPELVKPGTSVKISCKASGYSFTSYYIHWVKQSPGQGLEWIGWIYPGSGNTKYSEKFKGKATLTADTSSSTAYMQLSSLTSEDSAVYFCARDGCYGFAYWGQGTLVTVS

SEQ ID NO: 61 - protein, 111 aa, Mus musculus
DIVLTQSPASLAVSLGQRATISCKASQSVDYDGDSYMNWYQQKPGQPPKFLICAASNLESGIPARFSGSGSGTDFTLNIHPVEEEDAATYYCQQSNEDPFTFGSGTKLEIK

SEQ ID NO: 62 - protein, 115 aa, Oryctolagus cuniculus
QSVEESGGRLVTPGTPLTLTCTVSGFSINSYAMIWVRQAPGEGLEWIGVIYPSGNTYYANWAKGRFTVSRTSTTVDLKITSPTTEDTATYFCARRDGTDKTFNIWGPGTLVTVSL

SEQ ID NO: 63 - protein, 112 aa, Oryctolagus cuniculus
QVLTQTPSPVSAAVGGTVTINCQASQNVYGDNYLAWYQQKPGQPPKLLIYEASKLASGVPPRFSGSGSGTQFTLTISDVQCDDAATYYCQGEFLCTTSDCFTFGGGTGVVVR

SEQ ID NO: 64 - protein, 119 aa, Oryctolagus cuniculus
QSVEESGGRLVTPGTPLTLTCTVSGIDLSRYAMSWVRQAPGKGLEWIGVINSSGATYYASWAKGRFTISETSTTVELKITSPTTEDTATYFCARWTYDDYGDFQGFNIWGPGTLVTVSL

SEQ ID NO: 65 - protein, 111 aa, Oryctolagus cuniculus
AVLTQTPSPVSAAVGGTVTISCQSSQSVYNNNDLAWYQQKPGQPPKLLIYRASKLASGVPSRFKGSGSGTQFTLTISGVQCDDAATYYCLGGYDDDADMGAFGGGTEVVVK

SEQ ID NO: 66 - protein, 111 aa, synthetic construct
  REGION 1..111: note = heavy chain variable domain
QSLEESGGRLVTPGTPLTLTCTVSGIDLSRDTMIWVRQAPGEGLEWIGSIYTDSGNTWYASWVKGRFTISKTSSTTVDLRITSPTTEDTATYFCARNFSVWGPGTLVTVSL

SEQ ID NO: 67 - protein, 112 aa, synthetic construct
  REGION 1..112: note = light chain variable domain
QVLTQTPSPVSAAVGGTVTINCQASQSVYNSDRLAWFQQMRGQPPKLLIYDVSKLASGVPSRFSGSGSGTQFTLTISDVQCDDAATYYCLGGYDCSSAECNVFGGGTEVVVK

SEQ ID NO: 68 - protein, 122 aa, synthetic construct
  REGION 1..122: note = 299-023 VH humanization variant_DASG
QSMQESGPGLVKPSQTLSLTCTVSGFSLSSYAMSWIRQHPGKGLEWIGYIWSGGSTDYASWAKSRVTISKTSTTVSLKLSSVTAADTAVYYCARRYGTSYPDYGDASGFDPWGQGTLVTVSS

SEQ ID NO: 69 - protein, 4 aa, synthetic construct
AAAA

SEQ ID NO: 70 - protein, 4 aa, synthetic construct
AAAA

SEQ ID NO: 71 - protein, 4 aa, synthetic construct
AAAA

SEQ ID NO: 72 - protein, 110 aa, synthetic construct
  REGION 1..110: note = 299-009 VL humanization variant_NYA
AIQLTQSPSSLSASVGDRVTITCRASQSISSYLAWYQQKPGKAPKLLIYRASTLASGVPSRFSGSGSGTDFTLTISSLQPEDFATYYCQQNYASSNVDNTFGGGTKVEIK

SEQ ID NO: 73 - protein, 4 aa, synthetic construct
AAAA

SEQ ID NO: 74 - protein, 4 aa, synthetic construct
AAAA

SEQ ID NO: 75 - protein, 4 aa, synthetic construct
AAAA

SEQ ID NO: 76 - protein, 126 aa, Homo sapiens [sequence omitted]

SEQ ID NO: 77 - protein, 10 aa, synthetic construct
GFTFSSYAMS

SEQ ID NO: 78 - protein, 17 aa, synthetic construct
AINASGTRTYYADSVKG

SEQ ID NO: 79 - protein, 17 aa, synthetic construct
GKGNTHKPYGYVRYFDV

SEQ ID NO: 80 - protein, 108 aa, Homo sapiens [sequence omitted]

SEQ ID NO: 81 - protein, 12 aa, synthetic construct
RASQSVSSSYLA

SEQ ID NO: 82 - protein, 7 aa, synthetic construct
GASSRAT

SEQ ID NO: 83 - protein, 9 aa, synthetic construct
LQIYNMPIT

SEQ ID NO: 84 - protein, 122 aa, synthetic construct
QVELVESGGGLVQPGGSLRLSCAASGFTFSSYAMSWVRQAPGKGLEWVSAINATGTRTYYADSVKGRFTISRDNSKNTLYLQMNSLRAEDTAVYYCARGKGSSGYVRYFDVWGQGTLVTVSS

SEQ ID NO: 85 - protein, 10 aa, synthetic construct
GFTFSSYAMS

SEQ ID NO: 86 - protein, 17 aa, synthetic construct
AINATGTRTYYADSVKG

SEQ ID NO: 87 - protein, 13 aa, synthetic construct
GKGSSGYVRYFDV

SEQ ID NO: 88 - protein, 122 aa, synthetic construct
QVELVESGGGLVQPGGSLRLSCAASGFTFSSYAMSWVRQAPGKGLEWVSAIQASGTRTYYADSVKGRFTISRDNSKNTLYLQMNSLRAEDTAVYYCARGKGSSGYVRYFDVWGQGTLVTVSS

SEQ ID NO: 89 - protein, 17 aa, synthetic construct
AIQASGTRTYYADSVKG

SEQ ID NO: 90 - protein, 108 aa, synthetic construct
DIVLTQSPATLSLSPGERATLSCRASQSVSSSYLAWYQQKPGQAPRLLIYGASSRATGVPARFSGSGSGTDFTLTISSLEPEDFATYYCLQIYNHPITFGQGTKVEIK

SEQ ID NO: 91 - protein, 9 aa, synthetic construct
LQIYNHPIT

SEQ ID NO: 92 - protein, 122 aa, synthetic construct
EVQLVQSGAEVKKPGESLKISCKGSGYSFTTYWIGWVRQMPGKGLEWMGIIYPGDSDTRYSPSFQGQVTISADKSISTAYLQWSSLKASDTAMYYCARAGHYDGGHLGMDVWGQGTTVTVSS

SEQ ID NO: 93 - protein, 108 aa, synthetic construct
EIVMTQSPGTLSLSPGERATLSCRASQSVSSSYLAWYQQKPGQAPRLLIYGASNRATGIPDRFSGSGSGTDFTLTISRLEPEDFAVYYCQQDDSAPYTFGGGTKVEIK

SEQ ID NO: 94 - protein, 123 aa, synthetic construct
QVQLVQSGAEVKKPGASVKVSCKASGYTFTSYYIHWVRQAPGQGLEWMGIINPSGGSTSYAQKFQGRVTMTRDTSTSTVYMELSSLRSEDTAVYYCAREADDSSGYPLGLDVWGQGTMVTVSS

SEQ ID NO: 95 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQVNSLPPTFGGGTKVEIK

SEQ ID NO: 96 - protein, 116 aa, synthetic construct
EVKLEESGGGLVQPGGSMKLSCAASGFTFSDAWMGWVRQSPEKGLEWVAEIRDKVKNHATYYAESVKGRFTISRDDSKSTVYLQMNTLRADDTGIYYCRLGVFDYWGQGTTLTVSS

SEQ ID NO: 97 - protein, 112 aa, synthetic construct
DVVMTQTPLTLSVTIGQPASISCKSSQSLLYSNGKTFLSWLLQRPGQSPKRLIYLVSKLDSGVPDRFAGSGSGTDFTLKISRLEADDLGIYYCMQGTHFPLTFGAGTKLELK

SEQ ID NO: 98 - protein, 116 aa, synthetic construct
QVQLQQSGAELVKPGASVKLSCKASGYTVSRYWMHWVKQRPGRGLEWIGRIDPNSGGTKYNEKFKTKATLTVDKPSSTAYMQVSSLTSEDSAVYYCVLTGTDFDYWGQGTTLTVSS

SEQ ID NO: 99 - protein, 112 aa, synthetic construct
DVVMTQTPLSLPVSLGDQASFSCRSSQSLVHSNGNTYLHWFLQKPGQSPKLLIFKVSNRVSGVPDRFSGSGSGTDFTLKISRVEAEDLGVYFCSQSTHVPLTFGAGTKLELK

SEQ ID NO: 100 - protein, 123 aa, synthetic construct
QVQLQQSGPELVKPGASLKISCKASGYAFSSSWMNWVKQRPGKGLEWIGRIYPGDGDTNYNGEFRVRATLTADTSSTTAYMQLSSLTSEDSAVYFCARLLRNQPGESYAMDYWGQGASVTVSS

SEQ ID NO: 101 - protein, 112 aa, synthetic construct
DVVMTQTPLSLPVSLGDQASISCRSSQSLVHSNGYTYLHWYLQKPGQSPKLLIYKVSNRFSGVPDRFSGSGSGTDFTLKISRVEADDLGVYFCSQSTRVPYTFGGGTKLEIK

SEQ ID NO: 102 - protein, 115 aa, synthetic construct
EVQLVESGGGLVQPGGSLRLSCAASGFNLSYSSMHWVRQAPGKGLEWVASIYSSYGSTSYADSVKGRFTISADTSKNTAYLQMNSLRAEDTAVYYCARHYALDYWGQGTLVTVSS

SEQ ID NO: 103 - protein, 109 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQYSWYGYPITFGQGTKVEIK

SEQ ID NO: 104 - protein, 115 aa, synthetic construct
EVQLVESGGGLVQPGGSLRLSCAASGFNLSSSYMHWVRQAPGKGLEWVASIYPYYGYTSYADSVKGRFTISADTSKNTAYLQMNSLRAEDTAVYYCARHYAMDYWGQGTLVTVSS

SEQ ID NO: 105 - protein, 109 aa, synthetic construct
DIQMTQSPSSLSASVGDRVTITCRASQSVSSAVAWYQQKPGKAPKLLIYSASSLYSGVPSRFSGSRSGTDFTLTISSLQPEDFATYYCQQGYYASAPITFGQGTKVEIK

SEQ ID NO: 106 - protein, 123 aa, synthetic construct
QVQLVQSGAEVKKPGASVKVSCKASGYAFSSQWMNWVRQAPGQRLEWIGRIYPGGGDTNYAGKFQGRVTITADTSASTAYMELSSLRSEDTAVYYCARLLRNQPGESYAMDYWGQGTLVTVSS

SEQ ID NO: 107 - protein, 112 aa, synthetic construct
DVVMTQSPDSLAVSLGERATINCRSSQSLVHSNRYTYLHWYQQKPGQSPKLLIYKVSNRFSGVPDRFSGSGSGTDFTLKISRVEAEDVGVYYCSQSTRVPYTFGQGTKLEIK

SEQ ID NO: 108 - protein, 116 aa, synthetic construct
QVQLQQPGAELVKPGASVKLSCKASGYTFTSYWMHWVKQSPGRGLEWIGRSDPTTGGTNYNEKFKTKATLTVDKPSSTAYMQLSSLTSDDSAVYYCVRTSGTGDYWGQGTSLTVSS

SEQ ID NO: 109 - protein, 112 aa, synthetic construct
DVVMTQTPLSLPVSLGDQASISCRSSQSLVHNNGNTFLHWYLQKPGQSPKLLIYKVSNRFSGVPDRFSGSGSGTDFTLKISRVEAEDLGVYFCSQTTHVPPTFGGGTKLEIK

SEQ ID NO: 110 - protein, 123 aa, synthetic construct
EVQLVQSGAEVKKPGESLKISCKGSGYSFTSYWIAWVRQMPGKGLEWMGTIYPGDADARYSPSFQGQVTISADKSISTAYLQWSSLKASDTAMYFCARQRTFYYDSSDYFDYWGQGTLVTVSS

SEQ ID NO: 111 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYFCQQADAFPRTFGQGTKLEIK

SEQ ID NO: 112 - protein, 125 aa, synthetic construct
EVQLVQSGAEVKKPGESLKISCKGSGFSFNTYWIGWVRQMPGKGLEWMGIIYPGDQDIRYSPSFQGQVTISADKSISTAYLQWSSLKASDTAMYYCARYGRYIYGYGGYHGMDVWGQGTTVTVSS

SEQ ID NO: 113 - protein, 107 aa, synthetic construct
DIQMTQSPSSLSASVGDRVTITCRASQAIRDDLGWYQQKPGKAPKLLIYAASSLQSGVPSRFSGSGSGTDFTLTISSLQPEDFATYYCLQNYNYPHTFGQGTKLEIK

SEQ ID NO: 114 - protein, 119 aa, synthetic construct
EVQLVQSGAEVKKPGESLKISCKGSGYTFTDYNIHWVRQMPGKGLEWMGYIYPKNGGTGYTQKFKSQVTISVDNSISTAYLQWSSLKASDTAMYYCARRTARASWFAFWGQGTLVTVSS

SEQ ID NO: 115 - protein, 113 aa, synthetic construct
DIVMTQSPATLSVSPGERATLSCKSSQSLLYSSNQKNYLAWYQQKPGQAPRVLIYWASTRESGIPARFSGSGSGTEFTLTISSLQSEDFAVYYCQQYYNYPFTFGQGTKLEIK

SEQ ID NO: 116 - protein, 116 aa, Mus musculus
EVQLQQSGPDLVKPGASVKMSCKASGYTFTDYNIHWVKQSHGKTLEWIGYINPNTGGTYYNQKFKGKATMTVNKSSSTAYMELRSLTSEDSAVYYCVATRWDGVNWAQGTLVTVSA

SEQ ID NO: 117 - protein, 112 aa, Mus musculus
DVVMTQTPLSLPVSLGDQAFISCRSSQNLVHSNGNTYLHWYLQKPGQSPKLLIYIVSNRFSGVPDRFSGSGSGTDFTLEISRVEAEDLGVYFCSQSTHVPLTFGAGTKLELK

SEQ ID NO: 118 - protein, 119 aa, synthetic construct
QVQLVQSGAEVKKPGASVKVSCKASGYTFTGYHMSWVRQAPGQGLEWMGVINPVSGNTVYAQKFQGRVTMTRDTSISTAYMELSRLRSEDTAVYYCARIPSYTYAFDYWGQGTLVTVSS

SEQ ID NO: 119 - protein, 107 aa, synthetic construct
DIQMTQSPSSLSASVGDRVTITCRASQDISNYLAWYQQKPGKAPKLLIYRASSLQSGVPSRFSGSGSGTDFTLTISSLQPEDFATYYCQQHGHSPTTFGQGTKVEIK

(19) United States
(12) Patent Application Publication
(10) Pub. No.: US 2025/0051429 A1
(43) Pub. Date: Feb. 13, 2025

(54) ANTI-A-BETA PROTEIN ANTIBODIES, METHODS AND USES THEREOF

(71) Applicant: Hoffmann-La Roche Inc., Little Falls, NJ (US)

(72) Inventors: Per-Ola FRESKGARD, Norrköping (SE); Guy GEORGES, Habach (DE); Sabine IMHOF-JUNG, Krailling (DE); Markus NEUBAUER, Iffeldorf (DE); Jens NIEWOEHNER, Neuried (DE); Petra RUEGER, Penzberg (DE)

(21) Appl. No.: 18/797,664

(22) Filed: Aug. 8, 2024

(30) Foreign Application Priority Data
Aug. 9, 2023 (EP) 23190645.4

(51) Int. Cl. C07K 16/18; A61K 39/00

(52) U.S. Cl. CPC C07K 16/18; A61K 2039/505; C07K 2317/94

(57) ABSTRACT

Herein is reported an antibody that binds to human A-beta protein, wherein the antibody comprises a heavy chain variable domain (VH) and a light chain variable domain comprising CDRs selected from (1) CDRs of SEQ ID NO: 85, 86, 87, 81, 82 and 83; or (2) CDRs of SEQ ID NO: 85, 89, 87, 81, 82 and 83; or (3) CDRs of SEQ ID NO: 85, 86, 87, 81, 82 and 91; or (4) CDRs of SEQ ID NO: 85, 89, 87, 81, 82 and 91.

### CROSS-REFERENCE TO RELATED APPLICATIONS

This application claims priority from European Patent Application No. 23190645.4 filed on Aug. 9, 2023. The European Patent Application is hereby expressly incorporated by reference herein in its entirety.

The present invention relates to antibodies against the human A-beta protein (anti-A-beta protein antibodies), methods for their production, pharmaceutical compositions containing these antibodies, and uses thereof. The antibodies according to the current invention shown improved technical and biological properties compared to known anti-A-beta protein antibodies.

### INCORPORATION BY REFERENCE OF SEQUENCE LISTING

The instant application contains a Sequence Listing which has been submitted electronically in XML file format and is hereby incorporated by reference in its entirety. Said XML copy, created on Jul. 7, 2024, is named P38749-WO.xml and is 140,686 bytes in size.

### BACKGROUND OF THE INVENTION

About 70% of all cases of dementia are due to Alzheimer's disease (AD) that is associated with selective damage of brain regions and neural circuits critical for cognition. AD is characterized by neurofibrillary tangles in particular in pyramidal neurons of the hippocampus and numerous amyloid plaques containing mostly a dense core of amyloid deposits and defused halos.

The extracellular neuritic plaques contain large amounts of a pre-dominantly fibrillar peptide termed “amyloid”, “amyloid β”, “A-beta”, “Aβ4”, “β-A4” or “Aβ”; see Selkoe, Ann. Rev. Cell Biol. 10 (1994) 373-403; Koo, Proc. Natl. Acad. Sci. USA 96 (1999) 9989-9990; U.S. Pat. No. 4,666,829; Glenner BBRC 12 (1984) 1131). This amyloid is derived from “Alzheimer precursor protein/P-amyloid precursor protein” (APP). APPs are integral membrane glycoproteins (see Sisodia, Proc. Natl. Acad. Sci. USA 89 (1992) 6075) and are endoproteolytically cleaved within the AP sequence by a plasma membrane protease, α-secretase (see Sisodia (1992), Joe. cit.). Furthermore, further secretase activity, in particular β-secretase and γ-secretase activity leads to the extracellular release of amyloid-β (Aβ) comprising either 39 amino acids (Aβ39), 40 amino acids (Aβ40), 41 amino acids (Aβ41), 42 amino acids (Aβ42) or 43 amino acids (Aβ43) (see Sinha, Proc. Natl. Acad. Sci. 96 (1999) 11094-1053; Price, Science 282 (1998) 1078-1083; WO 00/72880 or Hardy, TINS 20 (1997) 154).

It is of note that the A-beta protein has several naturally occurring forms, whereby the human forms are referred to as Aβ39, Aβ40, Aβ41, Aβ42 and Aβ43 as mentioned above. The most prominent form, Aβ42, has the amino acid sequence (starting from the N-terminus): DAEFRHDSGYEVHHQKLVFFAEDVGSNKGAIIGLMVGGV VIA (SEQ ID NO: 45). In Aβ41, Aβ40, Aβ39, the C-terminal amino acids A, IA and VIA are missing, respectively. In Aβ43 an additional threonine residue is comprised at the C-terminus of the above depicted sequence.

The time required to nucleate Aβ40 fibrils was shown to be significantly longer than that to nucleate Aβ42 fibrils (see e.g. Lansbury, Jr., P. T. and Harper, J. D., Ann. Rev. Biochem. 66 (1997) 385-407). As reviewed in Wagner (J. Clin. Invest. 104 (1999) 1239-1332) Aβ42 is more frequently found associated with neuritic plaques and is considered to be more fibrillogenic in vitro. It was also suggested that Aβ42 serves as a “seed” in the nucleation-dependent polymerization of ordered non-crystalline AR peptides (see e.g. Jarrett, Cell 93 (1993) 1055-1058). Modified APP processing and/or the generation of extracellular plaques containing proteinaceous depositions are not only known from Alzheimer's pathology but also from subjects suffering from other neurological and/or neurodegenerative disorders. These disorders comprise, inter alia, Down's syndrome, hereditary cerebral hemorrhage with amyloidosis Dutch type, Parkinson's disease, ALS (amyotrophic lateral sclerosis), Creutzfeldt Jacob disease, HIV-related dementia and motor neuropathy.

Until now, only limited medical intervention schemes for amyloid-related diseases have been described. For example, cholinesterase inhibitors like galantamine, rivastigmine or donepezil have been discussed as being beneficial in Alzheimer's patients with only mild to moderate disease. However, adverse events have been reported due to cholinergic action of these drugs. While these cholinergic-enhancing treatments do produce some symptomatic benefit, therapeutic response is not satisfactory for the majority of patients treated. It has been estimated that significant cognitive improvement occurs in only about 5% of treated patients and there is little evidence that treatment significantly alters the course of this progressive disease.

Consequently, there remains a tremendous clinical need for more effective treatments and in particular those which may arrest or delay progression of the disease.

In addition, NMDA-receptor antagonists, like memantine, have been employed. However, adverse events have been reported due to the pharmacological activity. Further, such a treatment with these NMDA-receptor antagonists can merely be considered as a symptomatic approach and not a disease-modifying one.

In addition, immunomodulation approaches for the treatment of amyloid-related disorders have been proposed. WO 99/27944 discloses conjugates that comprise parts of the A-beta peptide and carrier molecules whereby said carrier molecule should enhance an immune response. Another active immunization approach is mentioned in WO 00/72880, wherein also A-beta fragments are employed to induce an immune response.

Further, passive immunization approaches with general anti-A-beta antibodies have been proposed in WO 99/27944 or WO 01/62801 and specific humanized antibodies directed against portions of A-beta have been described in WO 02/46237, WO 02/088306 and WO 02/088307. WO 00/77178 describes antibodies binding a transition state adopted by β-amyloid during hydrolysis. WO 03/070760 discloses antibody molecules that recognize two discontinuous amino acid sequences on the A-beta peptide.

WO 2014/033074 relates to blood-brain-barrier shuttles that bind receptors on the blood brain barrier and methods of using the same. Blood-brain-barrier drug delivery of IgG fusion proteins with a transferrin receptor monoclonal antibody have been reported by Pardridge, W., et al. (Exp. Opin. Drug Deliv. 12 (2015) 207-222). Yu, Y. J. et al. (Sci. Translat. Med. 6 (2014) 261ra154-261ra154). They reported that therapeutic bispecific antibodies cross the blood-brain-barrier in non-human primates. The disaggregation of amyloid plaque in brain of Alzheimer's disease transgenic mice with daily subcutaneous administration of a tetravalent bispecific antibody that targets the transferrin receptor and the Abeta amyloid peptide was reported by Sumbria, R. K., et al. (Mol. Pharm. 10 (2013) 3507-3513). Niewoehner, J., et al. (Neuron 81 (2014) 49-609 reported an increased brain penetration and potency of a therapeutic antibody using a monovalent molecular shuttle.

WO 2016/207240 reported anti-transferrin receptor antibodies with designed off-rates for the human transferrin receptor and their use as blood-brain-barrier shuttle module

WO 2017/055542 reported trivalent, bispecific antibodies against human CD20 and human transferrin receptor, methods for their production, pharmaceutical compositions containing these antibodies, and uses thereof.

WO 2007/068429 reported antibodies against amyloid beta with glycosylation in the variable region. The purified antibody molecule preparation being characterized in that at least one antigen binding site comprises a glycosylated asparagine (Asn) in the variable region of the heavy chain (VH). WO 2007/068429 reported a mixture of antibodies comprising one or two glycosylated antigen binding sites with a glycosylated asparagine (Asn) in the variable region of the heavy chain, i.e. mixtures of isoforms of antibodies that comprise a glycosylated Asn in the variable region of the heavy chain (VH).

WO 2017/055540 reported trivalent, bispecific antibodies against human A-beta and human transferrin receptor, methods for their production, pharmaceutical compositions containing these antibodies, and uses thereof.

EP 2 368 907 reported anti-Abeta antibodies and their use.

Edwards, E., et al., reported strategies to control therapeutic antibody glycosylation during bioprocessing (Biotechnol. Bioeng. 119 (2022) 1343-1358).

### SUMMARY OF THE INVENTION

Herein are reported antibodies against the human A-beta protein (anti-A-beta protein antibodies), methods for their production, pharmaceutical compositions containing these antibodies, and uses thereof.

The antibodies according to the current invention are variants of the anti-A-beta antibody gantenerumab. They have improved technical and biological properties compared to their parent antibody. The improvements encompass, amongst other things, improved production properties, such as an improved production titer, improved production yield and improved process robustness.

The current invention is based, at least in part, on the finding that by introducing a specific mutation in the heavy chain CDR2 and by shortening the heavy chain CDR3 the properties of gantenerumab have been improved. Without being bound by this theory, it is assumed that the introduced modification resulted in the improved properties by reducing glyco-occupation heterogeneity in the Fab and by removing a de-amidation hotspot and thereby reducing aggregation tendency of the parent antibody gantenerumab.

The current invention is based, at least in part, on the finding that the light chain of the parent antibody gantenerumab does not require modification in order to retain binding affinity and specificity when combined with the modified heavy chain according to the current invention.

The anti-A-beta protein antibodies according to the current invention have substantial and up to 100% glyco-occupation of the glycosylation site in the heavy chain CDR2.

The anti-A-beta protein antibodies according to the current invention have, amongst other things, improved properties in terms of target binding, developability properties, IHC/plaque binding and PK behavior.

The current invention encompasses at least the following embodiments:
1. An antibody that binds to human A-beta protein, wherein the antibody has one or more of the following properties
   specifically binds to a human A-beta protein of SEQ ID NO: 45;
   has a glycosylation site in the heavy chain CDR2 that has a glyco-occupancy of at least 95% as determined by CE-SDS;
   is free of species with any non-glycosylated glycosylation site;
   has an EC50 value for the human A-beta protein of SEQ ID NO: 45 of 0.5 nM or less;
   is binding to A-beta plaques in vitro and not binding to non-A-beta protein molecules in brain samples at a staining concentration of up to and including 1 μg/mL;
   is binding to A-beta plaques in vivo more strongly than gantenerumab;
   has a relative occupancy of plaques in vivo in the cortex and hippocampus of more than 0.2 determine in a one-way Anova;
   has a thermal stability (DLS T_{agg}/DLS Tₘ) of more than 68° C.
2. An antibody that binds to human A-beta protein, wherein the antibody comprises a heavy chain variable domain (VH) and a light chain variable domain comprising CDRs selected from
   (1)
   (a) a CDR-H1 comprising the amino acid sequence of SEQ ID NO: 85;
   (b) a CDR-H2 comprising the amino acid sequence of SEQ ID NO: 86;
   (c) a CDR-H3 comprising the amino acid sequence of SEQ ID NO: 87;
   (d) a CDR-L1 comprising the amino acid sequence of SEQ ID NO: 81;
   (e) a CDR-L2 comprising the amino acid sequence of SEQ ID NO: 82; and
   (f) a CDR-L3 comprising the amino acid sequence of SEQ ID NO: 83;
   or
   (2)
   (a) a CDR-H1 comprising the amino acid sequence of SEQ ID NO: 85;
   (b) a CDR-H2 comprising the amino acid sequence of SEQ ID NO: 89;
   (c) a CDR-H3 comprising the amino acid sequence of SEQ ID NO: 87;
   (d) a CDR-L1 comprising the amino acid sequence of SEQ ID NO: 81;
   (e) a CDR-L2 comprising the amino acid sequence of SEQ ID NO: 82; and
   (f) a CDR-L3 comprising the amino acid sequence of SEQ ID NO: 83;
   or
   (3)
   (a) a CDR-H1 comprising the amino acid sequence of SEQ ID NO: 85;
   (b) a CDR-H2 comprising the amino acid sequence of SEQ ID NO: 86;
   (c) a CDR-H3 comprising the amino acid sequence of SEQ ID NO: 87;
   (d) a CDR-L1 comprising the amino acid sequence of SEQ ID NO: 81;
   (e) a CDR-L2 comprising the amino acid sequence of SEQ ID NO: 82; and
   (f) a CDR-L3 comprising the amino acid sequence of SEQ ID NO: 91;
   or
   (4)
   (a) a CDR-H1 comprising the amino acid sequence of SEQ ID NO: 85;
   (b) a CDR-H2 comprising the amino acid sequence of SEQ ID NO: 89;
   (c) a CDR-H3 comprising the amino acid sequence of SEQ ID NO: 87;
   (d) a CDR-L1 comprising the amino acid sequence of SEQ ID NO: 81;
   (e) a CDR-L2 comprising the amino acid sequence of SEQ ID NO: 82; and
   (f) a CDR-L3 comprising the amino acid sequence of SEQ ID NO: 91.
3. The antibody according to embodiment 2, wherein the antibody has one or more of the following properties
   specifically binds to a human A-beta protein of SEQ ID NO: 45;
   has a glycosylation site in the heavy chain CDR2 that has a glyco-occupancy of at least 95% as determined by CE-SDS;
   is free of species with any non-glycosylated glycosylation site;
   has an EC50 value for the human A-beta protein of SEQ ID NO: 45 of 0.5 nM or less;
   is binding to A-beta plaques in vitro and not binding to non-A-beta protein molecules in brain samples at a staining concentration of up to and including 1 μg/mL;
   is binding to A-beta plaques in vivo more strongly than gantenerumab;
   has a relative occupancy in vivo of plaques in the cortex and hippocampus of more than 0.2 determine in a one-way Anova;
   has a Thermal stability (DLS T_{agg}/DLS Tₘ) of more than 68° C.
4. The antibody according to any one of embodiments 1 to 3, wherein the antibody is a monoclonal antibody.
5. The antibody according to any one of embodiments 1 to 4, wherein the antibody is a humanized or chimeric antibody.
6. The antibody according to any one of embodiments 1 to 5, wherein the antibody is an antibody fragment that binds to human A-beta protein.
7. The antibody according to any one of embodiments 1 to 6, wherein the antibody comprises
   (a) a VH sequence having at least 95% sequence identity to the amino acid sequence of SEQ ID NO: 84;
   (b) a VL sequence having at least 95% sequence identity to the amino acid sequence of SEQ ID NO: 80; or
   (c) a VH sequence as defined in (a) and a VL sequence as defined in (b).
8. The antibody according to any one of embodiments 1 to 7, wherein the antibody comprises a VH sequence of SEQ ID NO: 84 and a VL sequence of SEQ ID NO: 80.
8a. The antibody according to any one of embodiments 1 to 7, wherein the antibody comprises a VH sequence of SEQ ID NO: 84 with a pyroglutamic acid (pE) residue instead of a glutamine (Q) residue as first N-terminal amino acid residue and a VL sequence of SEQ ID NO: 80.
9. An antibody that specifically binds to human A-beta protein comprising a VH sequence of SEQ ID NO: 84 and a VL sequence of SEQ ID NO: 80.
10. The antibody according to any one of embodiments 1 to 9, wherein the antibody is
   a) a full-length antibody of the human subclass IgG1,
   b) a full-length antibody of the human subclass IgG4,
   c) a full-length antibody of the human subclass IgG1 with the mutations L234A, L235A and P329G,
   d) a full-length antibody of the human subclass IgG1 with the mutations L234A, L235A and P329G in both heavy chains and the mutations T366W and S354C in one heavy chain and the mutations T366S, L368A, Y407V and Y349C in the respective other heavy chain,
   e) a full-length antibody of the human subclass IgG1 with the mutations L234A, L235A and P329G in both heavy chains and the mutations T366W and Y349C in one heavy chain and the mutations T366S, L368A, Y407V and S354C in the respective other heavy chain,
   f) a full-length antibody of the human subclass IgG4 with the mutations T366W and S354C in one heavy chain and the mutations T366S, L368A, Y407V and Y349C in the respective other heavy chain,
   g) a full-length antibody of the human subclass IgG4 with the mutations T366W and Y349C in one heavy chain and the mutations T366S, L368A, Y407V and S354C in the respective other heavy chain,
   h) a full-length antibody of the human subclass IgG1 with the mutations L234A, L235A, P329G, 1253A, H310A and H435A in both heavy chains and the mutations T366W and S354C in one heavy chain and the mutations T366S, L368A, Y407V and Y349C in the respective other heavy chain,
   i) a full-length antibody of the human subclass IgG1 with the mutations L234A, L235A, P329G, 1253A, H310A and H435A in both heavy chains and the mutations T366W and Y349C in one heavy chain and the mutations T366S, L368A, Y407V and S354C in the respective other heavy chain,
   j) a full-length antibody of the human subclass IgG1 with the mutations L234A, L235A, P329G, M252Y, S254T and T256E in both heavy chains and the mutations T366W and S354C in one heavy chain and the mutations T366S, L368A, Y407V and Y349C in the respective other heavy chain,
   k) a full-length antibody of the human subclass IgG1 with the mutations L234A, L235A, P329G, M252Y, S254T and T256E in both heavy chains and the mutations T366W and Y349C in one heavy chain and the mutations T366S, L368A, Y407V and S354C in the respective other heavy chain, or
   l) a full-length antibody of the human subclass IgG1 with the mutations L234A, L235A, P329G, H310A, H433A and Y436A in both heavy chains and the mutations i) T366W, and ii) S354C or Y349C, in one heavy chain and the mutations i) T366S, L368A, and Y407V, and ii) Y349C or S354C, in the respective other heavy chain, or
   m) one of a) to 1) without the C-terminal lysine residue.
10a. The antibody according to embodiment 10, wherein the antibody mediates uptake of Abeta coated beads in vitro by iPSC-derived microglia.
10b. The antibody according to embodiment 10a, wherein the antibody has an Fc-region of c), d), e), h), i), j), k) or 1).
11. The antibody according to any one of embodiments 1 to 10b, wherein the antibody binds to human A-beta protein of SEQ ID NO: 45 with an affinity of 0.4 nM or less as determined/measured by surface plasmon resonance.
12. The antibody according to any one of embodiments 1 to 11, wherein the antibody is a full-length antibody of the human subclass IgG1.
13. The antibody according to any one of embodiments 1 to 12, wherein the antibody comprises i) a heavy chain comprising a heavy chain variable domain of SEQ ID NO: 84 and a heavy chain constant region of SEQ ID NO: 01, and ii) a light chain comprising a light chain variable domain of SEQ ID NO: 80 and a light chain kappa constant domain of SEQ ID NO: 29.
13a. The antibody according to any one of claims 1 to 12, wherein the antibody comprises a heavy chain comprising a heavy chain variable domain of SEQ ID NO: 84 with a pyroglutamic acid (pE) residue instead of a glutamine (Q) residue as first N-terminal amino acid residue and a heavy chain constant region of SEQ ID NO: 01 and a light chain comprising a light chain variable domain of SEQ ID NO: 80 and a light chain kappa constant domain of SEQ ID NO: 29.
14. The antibody according to any one of embodiments 1 to 13a, wherein the antibody is a multispecific antibody.
15. The antibody according to any one of embodiments 1 to 14, wherein the antibody is a multispecific antibody comprising at least one binding site binding to the human A-beta protein and at least one binding site not binding to the human A-beta protein/a second non-human A-beta protein target.
16. The antibody according to any one of embodiments 14 to 15, wherein the at least one binding site not binding to the human A-beta protein is binding to epidermal growth factor receptor (EGFR), human epidermal growth factor receptor 2 (HER2), alpha-synuclein, CD20, amyloid precursor protein (APP), glucocerebrosidase, human transferrin receptor 1, or human TREM2 protein.
17. The antibody according to any one of embodiments 14 to 16, wherein the multispecific antibody binds both
   i) human A-beta protein and human transferrin receptor, or
   ii) human A-beta protein and human TREM2 protein, or
   iii) human A-beta protein and human CD20 protein, or
   iv) human A-beta protein and human alpha-synuclein protein, or
   v) human A-beta protein and human phospho-tau protein, or
   vi) human A-beta protein and human glucocerebrosidase.
18. The antibody according to any one of embodiments 1 to 17, wherein the antibody is a bispecific antibody comprising
   i) a first binding site comprising a heavy chain variable domain of SEQ ID NO: 84 and a light chain variable domain of SEQ ID NO: 80, and
   ii) a second binding site selected from
      a) a heavy chain variable domain of SEQ ID NO: 68 and a light chain variable domain of SEQ ID NO: 72, or
      b) a heavy chain variable domain of SEQ ID NO: 54 and a light chain variable domain of SEQ ID NO: 55, or
      c) a heavy chain variable domain of SEQ ID NO: 56 and a light chain variable domain of SEQ ID NO: 57, or
      d) a heavy chain variable domain of SEQ ID NO: 58 and a light chain variable domain of SEQ ID NO: 59, or
      e) a heavy chain variable domain of SEQ ID NO: 60 and a light chain variable domain of SEQ ID NO: 61, or
      f) a heavy chain variable domain of SEQ ID NO: 62 and a light chain variable domain of SEQ ID NO: 63, or
      g) a heavy chain variable domain of SEQ ID NO: 64 and a light chain variable domain of SEQ ID NO: 65, or
      h) a heavy chain variable domain of SEQ ID NO: 66 and a light chain variable domain of SEQ ID NO: 67, or
      i) a heavy chain variable domain of SEQ ID NO: 92 and a light chain variable domain of SEQ ID NO: 93, or
      j) a heavy chain variable domain of SEQ ID NO: 94 and a light chain variable domain of SEQ ID NO: 95, or
      k) a heavy chain variable domain of SEQ ID NO: 96 and a light chain variable domain of SEQ ID NO: 97, or
      l) a heavy chain variable domain of SEQ ID NO: 98 and a light chain variable domain of SEQ ID NO: 99, or
      m) a heavy chain variable domain of SEQ ID NO: 100 and a light chain variable domain of SEQ ID NO: 101, or
      n) a heavy chain variable domain of SEQ ID NO: 102 and a light chain variable domain of SEQ ID NO: 103, or
      o) a heavy chain variable domain of SEQ ID NO: 104 and a light chain variable domain of SEQ ID NO: 105, or
      p) a heavy chain variable domain of SEQ ID NO: 106 and a light chain variable domain of SEQ ID NO: 107, or
      q) a heavy chain variable domain of SEQ ID NO: 108 and a light chain variable domain of SEQ ID NO: 109, or
      r) a heavy chain variable domain of SEQ ID NO: 110 and a light chain variable domain of SEQ ID NO: 111, or
      s) a heavy chain variable domain of SEQ ID NO: 112 and a light chain variable domain of SEQ ID NO: 113, or
      t) a heavy chain variable domain of SEQ ID NO: 114 and a light chain variable domain of SEQ ID NO: 115, or
      u) a heavy chain variable domain of SEQ ID NO: 116 and a light chain variable domain of SEQ ID NO: 117, or
      v) a heavy chain variable domain of SEQ ID NO: 118 and a light chain variable domain of SEQ ID NO: 119.
18a. The antibody according to any one of embodiments 1 to 18, wherein the antibody specifically binds to human A-beta protein, or wherein the antibody specifically binds to human A-beta protein and human TREM2.
18b. The antibody according to any one of embodiments 1 to 18a, wherein the antibody is a bivalent antibody.
18c. The antibody according to any one of embodiments 14 to 18b, wherein the antibody is a bivalent or trivalent or tetravalent antibody.
18d. The antibody according to any one of embodiments 14 to 18c, wherein the antibody is a bivalent, bispecific antibody.
18e. The antibody according to embodiment 18d, wherein the antibody is a bivalent, bispecific antibody in CrossMab format.
18f. The antibody according to any one of embodiments 14 to 18e, wherein the antibody binds to human A-beta protein and human TREM2 protein.
18g. The antibody according to any one of embodiments 14 to 18e, wherein the antibody binds to human A-beta protein and human transferrin receptor 1.
18h. The antibody according to any one of embodiments 14 to 18c, wherein the antibody is a trivalent, bispecific antibody.
18i. The antibody according to embodiment 18h, wherein the antibody is a trivalent, bispecific antibody in BS-format or TCB-format
18j. The antibody according to any one of embodiments 14 to 18c and 18g to 18h, wherein the antibody binds to human A-beta protein and human transferrin receptor 1.
18k. The antibody according to any one of embodiments 14 to 18c, wherein the antibody is a tetravalent, bispecific antibody.
18l. The antibody according to embodiment 18k, wherein the antibody is a tetravalent, bispecific antibody in 2+2-format.
18m. The antibody according to any one of embodiments 14 to 18c and 18k to 18l, wherein the antibody binds to human A-beta protein and human TREM2 protein.
18n. The antibody according to any one of embodiments 14 to 18c and 18k to 18l, wherein the antibody binds to human A-beta protein and human transferrin receptor 1.
18o. The antibody according to any one of embodiments 14 to 18c, wherein the antibody is a trivalent, trispecific antibody, wherein the antibody is binding to human A-beta protein, human transferrin receptor 1 and a third non-human A-beta and non-human transferrin receptor 1 target.
18p. The antibody according to any one of embodiments 14 to 18c and 18o, wherein the antibody is a trivalent, trispecific antibody, wherein the antibody is binding to human A-beta protein, human transferrin receptor 1 and human TREM2.
18q. The antibody according to any one of embodiments 18o to 18p, wherein the antibody is a trivalent, trispecific antibody in BS-format.
18r. The antibody according to any one of embodiments 18o to 18p, wherein the antibody is a trivalent, trispecific antibody in TCB format.
19. An immunoconjugate comprising the antibody according to any one of embodiments 1 to 18r and a cytotoxic agent.
20. An isolated nucleic acid molecule encoding the antibody according to any of embodiments 1 to 18r.
21. A composition of isolated nucleic acid molecules wherein each of the isolated nucleic acid molecules of the composition encodes one chain of the antibody according to any one of embodiments 1 to 18r.
22. A host cell comprising the nucleic acid molecule of embodiment 20 or the composition of nucleic acid molecules of embodiment 21.
23. A method of producing an antibody that binds to human A-beta protein comprising culturing the host cell of embodiment 22 in a cultivation medium under conditions suitable for the expression of the antibody.
24. The method according to embodiment 23, further comprising the step of recovering the antibody from the host cell or/and the cultivation medium.
25. The method according to any one of embodiments 23 to 24, further comprising the step of purifying the antibody after recovery from the cell or/and the cultivation medium using one or more chromatography steps.
26. An antibody produced by a method according to any one of embodiments 23 to 25.
27. A pharmaceutical composition comprising the antibody according to any one of embodiments 1 to 18r or an immunoconjugate of embodiment 19 and a pharmaceutically acceptable carrier.
28. The pharmaceutical composition of embodiment 27, further comprising an additional therapeutic agent.
28a. The pharmaceutical composition of embodiment 28, wherein the additional therapeutic agent is an anti-TREM2 antibody.
28b. The pharmaceutical composition according to any one of embodiments 27 to 28a, wherein the pharmaceutical composition is for treatment of Alzheimer's disease.
28c. The pharmaceutical composition according to any one of embodiments 27 to 28a, wherein the pharmaceutical composition is for treatment of Parkinson's disease.
29. The antibody according to any one of embodiments 1 to 18r or the immunoconjugate according to embodiment 19 or the pharmaceutical composition of any one of embodiments 27 to 28c for use as a medicament.
29a. The antibody according to embodiment 29, wherein the medicament is for treatment of Alzheimer's disease.
29b. The antibody according to embodiment 29, wherein the medicament is for treatment of Parkinson's disease.
30. The antibody according to any one of embodiments 1 to 18r or the immunoconjugate according to embodiment 19 or the pharmaceutical composition of any one of embodiments 27 to 28c for use in treating a disease in an individual having a disease associated with amyloidogenesis in the brain.
31. The antibody according to any one of embodiments 1 to 18r or the immunoconjugate according to embodiment 19 or the pharmaceutical composition of any one of embodiments 27 to 28c for use in treating a disease in an individual having a disease associated with amyloid plaque formation in the brain.
32. The antibody according to any one of embodiments 1 to 18r or the immunoconjugate according to embodiment 19 or the pharmaceutical composition of any one of embodiments 27 to 28c for use in treating an amyloid disease or disorder in the brain.
33. The antibody according to any one of embodiments 30 to 32, wherein the disease is selected from the group consisting of dementia, Alzheimer's disease, motor neuropathy, Down's syndrome, Creutzfeldt Jacob disease, hereditary cerebral hemorrhage with amyloidosis Dutch type, Parkinson's disease, HIV-related dementia, ALS or neuronal disorders related to aging.
34. The antibody according to any one of embodiments 30 to 32, wherein the disease is Alzheimer's disease.
34a. The antibody according to any one of embodiments 30 to 32, wherein the disease is Parkinson's disease.
35. The antibody according to any one of embodiments 1 to 18r or the immunoconjugate according to embodiment 19 or the pharmaceutical composition of any one of embodiments 27 to 28c for use in inhibiting the formation of amyloid plaques in the brain.
36. The antibody according to any one of embodiments 1 to 18r or the immunoconjugate according to embodiment 19 or the pharmaceutical composition of any one of embodiments 27 to 28c for use in slowing down the formation of amyloid plaques in the brain.
37. The antibody according to any one of embodiments 1 to 18r or the immunoconjugate according to embodiment 19 or the pharmaceutical composition of any one of embodiments 27 to 28c for use in the prevention of amyloidogenesis in the brain.
38. The antibody according to any one of embodiments 1 to 18r or the immunoconjugate according to embodiment 19 or the pharmaceutical composition of any one of embodiments 27 to 28c for use in the prevention of amyloid plaque formation in the brain.
39. The antibody according to any one of embodiments 1 to 18r or the immunoconjugate according to embodiment 19 or the pharmaceutical composition of any one of embodiments 27 to 28c for use in reducing amyloid plaques in the brain
40. The antibody according to any one of embodiments 1 to 18r or the immunoconjugate according to embodiment 19 or the pharmaceutical composition of any one of embodiments 27 to 28c for use in removing amyloid plaques in the brain.
41. The antibody according to any one of embodiments 1 to 18r or the immunoconjugate according to embodiment 19 or the pharmaceutical composition of any one of embodiments 27 to 28c for use in disintegrating amyloid plaques in the brain.
42. The antibody according to any one of embodiments 1 to 18r or the immunoconjugate according to embodiment 19 or the pharmaceutical composition of any one of embodiments 27 to 28c for use in inhibiting the formation of amyloid plaques in the brain.
43. The antibody according to any one of embodiments 1 to 18r or the immunoconjugate according to embodiment 19 or the pharmaceutical composition of any one of embodiments 27 to 28c for use in disintegrating amyloid plaques in the brain.
44. Use of the antibody according to any one of embodiments 1 to 18r or the immunoconjugate according to embodiment 19 or the pharmaceutical composition of any one of embodiments 27 to 28c for the manufacture of a medicament.
45. The use according to embodiment 44, wherein the medicament is for treating a disease associated with amyloidogenesis in the brain in an individual.
46. The use according to embodiment 44, wherein the medicament is for treating a disease associated with amyloid plaque formation in the brain in an individual.
47. The use according to embodiment 44, wherein the medicament is for treating an amyloid disease or disorder in the brain.
48. The use according to embodiment 47, wherein the amyloid disease is selected from the group consisting of dementia, Alzheimer's disease, motor neuropathy, Down's syndrome, Creutzfeldt Jacob disease, hereditary cerebral hemorrhage with amyloidosis Dutch type, Parkinson's disease, HIV-related dementia, ALS or neuronal disorders related to aging.
49. The use according to any one of embodiments 47 or 48, wherein the medicament is for treatment of Alzheimer's disease.
49a. The use according to any one of embodiments 47 or 48, wherein the medicament is for treatment of Parkinson's disease.
50. The use according to embodiment 44, wherein the medicament is for inhibiting the formation of amyloid plaques in the brain.
51. The use according to embodiment 44, wherein the medicament is for slowing down the formation of amyloid plaques in the brain.
52. The use according to embodiment 44, wherein the medicament is for preventing amyloidogenesis in the brain.
53. The use according to embodiment 44, wherein the medicament is for preventing amyloid plaque formation in the brain.
54. The use according to embodiment 44, wherein the medicament is for reducing amyloid plaques in the brain 55. The use according to embodiment 44, wherein the medicament is for removing amyloid plaques in the brain.
56. The use according to embodiment 44, wherein the medicament is for disintegrating amyloid plaques in the brain.
57. The use according to embodiment 44, wherein the medicament is for inhibiting the formation of amyloid plaques in the brain.
58. The use according to embodiment 44, wherein the medicament is for disintegrating amyloid plaques in the brain.
59. Use of the antibody according to any one of embodiments 1 to 18r or the immunoconjugate according to embodiment 19 or the pharmaceutical composition of any one of embodiments 27 to 28c for the treatment of a disease in an individual having a disease associated with amyloidogenesis in the brain.
60. Use of the antibody according to any one of embodiments 1 to 18r or the immunoconjugate according to embodiment 19 or the pharmaceutical composition of any one of embodiments 27 to 28c for the treatment of a disease in an individual having a disease associated with amyloid-plaque formation in the brain.
61. Use of the antibody according to any one of embodiments 1 to 18r or the immunoconjugate according to embodiment 19 or the pharmaceutical composition of any one of embodiments 27 to 28c for treating an amyloid disease or disorder in the brain.
62. Use of the antibody according to embodiment 61, wherein the amyloid disease is selected from the group consisting of dementia, Alzheimer's disease, motor neuropathy, Down's syndrome, Creutzfeldt Jacob disease, hereditary cerebral hemorrhage with amyloidosis Dutch type, Parkinson's disease, HIV-related dementia, ALS or neuronal disorders related to aging.
63. Use of the antibody according to any one of embodiments 61 to 62, wherein the medicament is for treatment of Alzheimer's disease.
63a. Use of the antibody according to any one of embodiments 61 to 62, wherein the medicament is for treatment of Parkinson's disease.
64. Use of the antibody according to any one of embodiments 1 to 18r or the immunoconjugate according to embodiment 19 or the pharmaceutical composition of any one of embodiments 27 to 28c for inhibiting the formation of amyloid plaques in the brain.
65. Use of the antibody according to any one of embodiments 1 to 18r or the immunoconjugate according to embodiment 19 or the pharmaceutical composition of any one of embodiments 27 to 28c for slowing down the formation of amyloid plaques in the brain.
66. Use of the antibody according to any one of embodiments 1 to 18r or the immunoconjugate according to embodiment 19 or the pharmaceutical composition of any one of embodiments 27 to 28c for the prevention of amyloidogenesis in the brain.
67. Use of the antibody according to any one of embodiments 1 to 18r or the immunoconjugate according to embodiment 19 or the pharmaceutical composition of any one of embodiments 27 to 28c for the prevention of amyloid plaque formation in the brain.
68. Use of the antibody according to any one of embodiments 1 to 18r or the immunoconjugate according to embodiment 19 or the pharmaceutical composition of any one of embodiments 27 to 28c for reducing amyloid plaques in the brain
69. Use of the antibody according to any one of embodiments 1 to 18r or the immunoconjugate according to embodiment 19 or the pharmaceutical composition of any one of embodiments 27 to 28c for removing amyloid plaques in the brain.
70. Use of the antibody according to any one of embodiments 1 to 18r or the immunoconjugate according to embodiment 19 or the pharmaceutical composition of any one of embodiments 27 to 28c for disintegrating amyloid plaques in the brain.
71. Use of the antibody according to any one of embodiments 1 to 18r or the immunoconjugate according to embodiment 19 or the pharmaceutical composition of any one of embodiments 27 to 28c for inhibiting the formation of amyloid plaques in the brain.
72. Use of the antibody according to any one of embodiments 1 to 18r or the immunoconjugate according to embodiment 19 or the pharmaceutical composition of any one of embodiments 27 to 28c for disintegrating amyloid plaques in the brain.
73. A method of treating an individual having amyloid plaques in the brain comprising administering to the individual an effective amount of the antibody according to any one of embodiments 1 to 18r or the immunoconjugate according to embodiment 19 or the pharmaceutical composition of any one of embodiments 27 to 28c.
74. A method of treating an individual having amyloidogenesis in the brain comprising administering to the individual an effective amount of the antibody according to any one of embodiments 1 to 18r or the immunoconjugate according to embodiment 19 or the pharmaceutical composition of any one of embodiments 27 to 28c.
75. A method of treating an individual predicted to have or develop amyloid plaque formation in the brain comprising administering to the individual an effective amount of the antibody according to any one of embodiments 1 to 18r or the immunoconjugate according to embodiment 19 or the pharmaceutical composition of any one of embodiments 27 to 28c.
76. The method according to any one of embodiments 73 to 75 further comprising administering an additional therapeutic agent to the individual.
76a. The method according to embodiment 76, wherein the additional therapeutic agent is an anti-TREM2 antibody.
77. A method of treating a disease associated with amyloidogenesis in an individual comprising administering to the individual an effective amount of the antibody according to any one of embodiments 1 to 18r or the immunoconjugate according to embodiment 19 or the pharmaceutical composition of any one of embodiments 27 to 28c to treat a disease associated with amyloidogenesis.
78. A method of treating a disease associated with amyloid-plaque formation in the brain in an individual comprising administering to the individual an effective amount of the antibody according to any one of embodiments 1 to 18r or the immunoconjugate according to embodiment 19 or the pharmaceutical composition of any one of embodiments 27 to 28c to treat a disease associated with amyloid plaque formation.
79. A method of treating an amyloid disease or disorder in the brain in an individual comprising administering to the individual an effective amount of the antibody according to any one of embodiments 1 to 18r or the immunoconjugate according to embodiment 19 or the pharmaceutical composition of any one of embodiments 27 to 28c to treat a disease or disorder in the brain.
80. The method of treating according to embodiment 79, wherein the amyloid disease or disorder in the brain is selected from the group consisting of dementia, Alzheimer's disease, motor neuropathy, Down's syndrome, Creutzfeldt Jacob disease, hereditary cerebral hemorrhage with amyloidosis Dutch type, Parkinson's disease, HIV-related dementia, ALS or neuronal disorders related to aging.
81. The method of treating according to any one of embodiments 79 to 80, wherein the disease is Alzheimer's disease.
81a. The method of treating according to any one of embodiments 79 to 80, wherein the disease is Parkinson's disease.
82. A method of treating a disease associated with amyloidogenesis in an individual comprising administering to the individual an effective amount of the antibody according to any one of embodiments 1 to 18r or the immunoconjugate according to embodiment 19 or the pharmaceutical composition of any one of embodiments 27 to 28c to treat a disease associated with amyloidogenesis.

In addition to the various embodiments depicted and claimed, the disclosed subject matter is also directed to other embodiments having other combinations of the features disclosed or claimed herein. As such, the particular features presented herein can be combined with each other in other manners within the scope of the disclosed subject matter such that the disclosed subject matter includes any suitable combination of the features disclosed herein. The foregoing description of specific embodiments of the disclosed subject matter has been presented for purposes of illustration and description. It is not intended to be exhaustive or to limit the disclosed subject matter to those embodiments disclosed.

### DETAILED DESCRIPTION OF THE INVENTION

The current invention is based, at least in part, on the finding that by introducing a specific mutation in the heavy chain CDR2 and by shortening the heavy chain CDR3 the properties of the antibody gantenerumab have been improved. Without being bound by this theory, it is assumed that the introduced modification resulted in the improved properties by reducing glyco-occupation heterogeneity in the Fab and by removing a de-amidation hotspot and thereby reducing aggregation tendency of the parent antibody gantenerumab.

The current invention is based, at least in part, on the finding that the light chain of the parent antibody gantenerumab does not require modification in order to restore binding affinity and specificity when combined with the modified heavy chain according to the current invention.

### Definitions

Unless otherwise defined herein, scientific and technical terms used in connection with the current invention shall have the meanings that are commonly understood by those of ordinary skill in the art. Further, unless otherwise required by context, singular terms shall include pluralities and plural terms shall include the singular.

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, MD (1991) and is referred to as “numbering according to Kabat” herein. Specifically, the Kabat numbering system (see pages 647-660) of Kabat, et al., Sequences of Proteins of Immunological Interest, 5th ed., Public Health Service, National Institutes of Health, Bethesda, MD (1991) is used for the light chain constant domain CL of kappa and lambda isotype, and the Kabat EU index numbering system (see pages 661-723) is used for the constant heavy chain domains (CHI, Hinge, CH2 and CH3, which is herein further clarified by referring to “numbering according to Kabat EU index” in this case).

The knobs into holes dimerization modules and their use in antibody engineering are described in Carter P.; Ridgway J. B. B.; Presta L. G.: Immunotechnology, Volume 2, Number 1, February 1996, pp. 73-73(1).

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991).

Useful methods and techniques for carrying out the current invention are described in e.g. Ausubel, F. M. (ed.), Current Protocols in Molecular Biology, Volumes I to III (1997); Glover, N. D., and Hames, B. D., ed., DNA Cloning: A Practical Approach, Volumes I and 11 (1985), Oxford University Press; Freshney, R. I. (ed.), Animal Cell Culture—a practical approach, IRL Press Limited (1986); Watson, J. D., et al., Recombinant DNA, Second Edition, CHSL Press (1992); Winnacker, E. L., From Genes to Clones; N.Y., VCH Publishers (1987); Celis, J., ed., Cell Biology, Second Edition, Academic Press (1998); Freshney, R. I., Culture of Animal Cells: A Manual of Basic Technique, second edition, Alan R. Liss, Inc., N.Y. (1987).

The use of recombinant DNA technology enables the generation of derivatives of a nucleic acid. Such derivatives can, for example, be modified in individual or several nucleotide positions by substitution, alteration, exchange, deletion or insertion. The modification or derivatization can, for example, be carried out by means of site directed mutagenesis. Such modifications can easily be carried out by a person skilled in the art (see e.g. Sambrook, J., et al., Molecular Cloning: A laboratory manual (1999) Cold Spring Harbor Laboratory Press, New York, USA; Hames, B. D., and Higgins, S. G., Nucleic acid hybridization—a practical approach (1985) IRL Press, Oxford, England).

It must be noted that as used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. It is also to be noted that the terms “comprising”, “including”, and “having” can be used interchangeably.

The term “about” denotes a range of +/−20% of the thereafter following numerical value. In one embodiment the term about denotes a range of +/−10% of the thereafter-following numerical value. In one embodiment the term about denotes a range of +/−5% of the thereafter-following numerical value.

The term “amyloid plaque” denotes aggregates of misfolded proteins that form in the spaces between nerve cells. These abnormally configured proteins are thought to play a central role in Alzheimer's disease. The amyloid plaques first develop in the areas of the brain concerned with memory and other cognitive functions.

The term “determine” as used herein encompasses also the terms measure and analyze.

The term “comprising” also includes the term “consisting of”.

The term “anti-(human) A-beta protein antibody” refers to an antibody that is capable of binding the human A-beta protein with sufficient affinity such that the antibody is useful as a diagnostic and/or therapeutic agent in targeting human A-beta protein.

It is of note that the human A-beta protein has several naturally occurring forms, whereby the human forms are referred to as Aβ39, Aβ40, Aβ41, Aβ42 and Aβ43. The most prominent form, Aβ42, has the amino acid sequence of SEQ ID NO: 45. In Aβ41, Aβ40, Aβ39, the C-terminal amino acids A, IA and VIA are missing, respectively. In Aβ43 an additional threonine residue is comprised at the C-terminus of SEQ ID NO: 45. In one preferred embodiment, the antibody according to the invention specifically binds to the human A-beta protein that has the amino acid sequence of SEQ ID NO: 45.

The “central nervous system” or “CNS” refers to the complex of nerve tissues that control bodily function, and includes the brain and spinal cord.

The term “antibody” is used herein in a broad sense and encompasses various antibody structures, including but not limited to monoclonal antibodies, monospecific antibodies, multispecific antibodies (e.g. bispecific antibodies, trispecific antibodies) and fragments thereof so long as they exhibit the desired human A-beta protein binding activity.

As detailed above, the modified antibody according to the invention may be a bi- or multispecific antibody. In certain embodiments, the antibody according to the invention is a bispecific antibody in one of the following formats:
full-length antibody with domain exchange (CrossMab-format):
a multispecific IgG antibody comprising a first Fab fragment and a second Fab fragment, wherein in the first Fab fragment
a) only the CH1 and CL domains are replaced by each other (i.e. the light chain of the first Fab fragment comprises a VL and a CH1 domain and the heavy chain of the first Fab fragment comprises a VH and a CL domain);
b) only the VH and VL domains are replaced by each other (i.e. the light chain of the first Fab fragment comprises a VH and a CL domain and the heavy chain of the first Fab fragment comprises a VL and a CH1 domain); or
c) the CH1 and CL domains are replaced by each other and the VH and VL domains are replaced by each other (i.e. the light chain of the first Fab fragment comprises a VH and a CH1 domain and the heavy chain of the first Fab fragment comprises a VL and a CL domain); and
wherein the second Fab fragment comprises a light chain comprising a VL and a CL domain, and a heavy chain comprising a VH and a CH1 domain; wherein the full-length antibody with domain exchange comprises a first heavy chain including a CH3 domain and a second heavy chain including a CH3 domain, wherein both CH3 domains are engineered in a complementary manner by respective amino acid substitutions, in order to support heterodimerization of the first heavy chain and the modified second heavy chain;
full-length antibody with additional heavy chain C-terminal binding site (BS-format):
a multispecific IgG antibody comprising
a) one full-length antibody comprising two pairs each of a full-length antibody light chain and a full-length antibody heavy chain, wherein the binding sites formed by each of the pairs of the full-length heavy chain and the full-length light chain specifically bind to a first antigen or one is binding to a first antigen and the other is binding to a second antigen, and
b) one additional Fab fragment, wherein the additional Fab fragment is fused to the C-terminus of one of the heavy chains of the full-length antibody,
wherein the binding site of the additional Fab fragment specifically binds to a second antigen or in case the full-length antibody is binding to a first and a second antigen the additional Fab is binding to a third antigen,
wherein the additional Fab fragment specifically binding to the second antigen i) comprises a domain crossover such that a) the light chain variable domain (VL) and the heavy chain variable domain (VH) are replaced by each other, or b) the light chain constant domain (CL) and the heavy chain constant domain (CH1) are replaced by each other, or ii) is a single chain Fab fragment,
wherein the full-length antibody comprises a first heavy chain including a CH3 domain and a second heavy chain including a CH3 domain, wherein both CH3 domains are engineered in a complementary manner by respective amino acid substitutions, in order to support heterodimerization of the first heavy chain and the modified second heavy chain;
full-length antibody with two additional heavy chain C-terminal binding site (2+2-format):
a multispecific IgG antibody comprising
   a) one full-length antibody comprising two pairs each of a full-length antibody light chain and a full-length antibody heavy chain, wherein the binding sites formed by each of the pairs of the full-length heavy chain and the full-length light chain specifically bind to a first antigen, and
   b) two additional Fab fragment, wherein to each C-terminus of the heavy chains of the full-length antibody one additional Fab fragment is fused, wherein the binding sites of the additional Fab fragment specifically bind to a second antigen;
full-length antibody with additional heavy chain N-terminal site (TCB-format): a multispecific IgG antibody comprising
   a) one full-length antibody comprising two pairs each of a full-length antibody light chain and a full-length antibody heavy chain, wherein the binding sites formed by each of the pairs of the full-length heavy chain and the full-length light chain specifically bind to a first antigen or one is binding to a first antigen and the other is binding to a second antigen, and
   b) one additional Fab fragment, wherein the additional Fab fragment is inserted between one of the Fab fragments of the full-length antibody and the Fc-region, wherein the binding site of the additional Fab fragment specifically binds to a second antigen or in case the full-length antibody is binding to a first and a second antigen the additional Fab is binding to a third antigen,
wherein the additional Fab fragment specifically binding to the second antigen comprises a domain crossover such that a) the light chain variable domain (VL) and the heavy chain variable domain (VH) are replaced by each other, or b) the light chain constant domain (CL) and the heavy chain constant domain (CHI) are replaced by each other;
the one-armed single chain format (=one-armed single chain antibody): antibody comprising a first binding site that specifically binds to a first epitope or antigen and a second binding site that specifically binds to a second epitope or antigen, whereby the individual chains are as follows
   light chain (variable light chain domain+light chain kappa constant domain)
   combined light/heavy chain (variable light chain domain+light chain constant domain+peptidic linker+variable heavy chain domain+CH1+Hinge+CH2+CH3 with knob mutation)
   heavy chain (variable heavy chain domain+CH1+Hinge+CH2+CH3 with hole mutation);
the two-armed single chain format (=two-armed single chain antibody): antibody comprising a first binding site that specifically binds to a first epitope or antigen and a second binding site that specifically binds to a second epitope or antigen, whereby the individual chains are as follows
   combined light/heavy chain 1 (variable light chain domain+light chain constant domain+peptidic linker+variable heavy chain domain+CH1+Hinge+CH2+CH3 with hole mutation)
   combined light/heavy chain 2 (variable light chain domain+light chain constant domain+peptidic linker+variable heavy chain domain+CH1+Hinge+CH2+CH3 with knob mutation);
the common light chain bispecific format (=common light chain bispecific antibody):
antibody comprising a first binding site that specifically binds to a first epitope or antigen and a second binding site that specifically binds to a second epitope or antigen, whereby the individual chains are as follows
   light chain (variable light chain domain+light chain constant domain)
   heavy chain 1 (variable heavy chain domain+CH1+Hinge+CH2+CH3 with hole mutation)
   heavy chain 2 (variable heavy chain domain+CH1+Hinge+CH2+CH3 with knob mutation).

As used herein the term “replaced by each other” with respect to corresponding heavy and light chain domains refers to the aforementioned domain crossovers. As such, when CH1 and CL domains are “replaced by each other” it is referred to the domain crossover mentioned under item (i) and the resulting heavy and light chain domain sequence. Accordingly, when VH and VL are “replaced by each other” it is referred to the domain crossover mentioned under item (ii); and when the CH1 and CL domains are “replaced by each other” and the VH and VL domains are “replaced by each other” it is referred to the domain crossover mentioned under item (iii).

An “antibody fragment” refers to a molecule other than an intact antibody that comprises a portion of an intact antibody and that binds the same antigen to which the intact antibody binds. Examples of antibody fragments include but are not limited to Fv, Fab, Fab′, Fab′-SH, F(ab′)2; diabodies; linear antibodies; single-chain antibody molecules (e.g. scFv); DutaFab (bispecific Fabs) and multispecific antibodies formed from antibody fragments.

“Affinity” refers to the strength of the sum of all non-covalent interactions between a single binding site of a molecule (e.g., an antibody) and its binding partner (e.g., an antigen). Unless indicated otherwise, as used herein, “binding affinity” refers to intrinsic binding affinity that reflects a 1:1 interaction between members of a binding pair (e.g., antibody and antigen). Affinity can be measured by common methods known in the art, including those described herein.

The term “antibody-dependent cellular cytotoxicity (ADCC)” is a function mediated by Fc receptor binding and refers to lysis of target cells by an antibody as reported herein in the presence of effector cells. ADCC can be measured by the treatment of a preparation of CD19 expressing erythroid cells (e.g. K562 cells expressing recombinant human CD19) with an antibody according to the current invention in the presence of effector cells such as freshly isolated PBMC (peripheral blood mononuclear cells) or purified effector cells from buffy coats, like monocytes or NK (natural killer) cells. Target cells are labeled with 51Cr and subsequently incubated with the antibody. The labeled cells are incubated with effector cells and the supernatant is analyzed for released 51Cr. Controls include the incubation of the target endothelial cells with effector cells but without the antibody. The capacity of the antibody to induce the initial steps mediating ADCC is investigated by measuring their binding to Fcγ receptors expressing cells, such as cells, recombinantly expressing FcγRI and/or FcγRIIA or NK cells (expressing essentially FcγRIIIA).

A “multispecific antibody” denotes an antibody that has binding specificities for at least two different epitopes on the same antigen or two different antigens. Multispecific antibodies can be prepared as full-length antibodies or antibody fragments (e.g. F(ab′)2 bispecific antibodies or DutaFabs) or combinations thereof (e.g. full-length antibody plus additional/fused one or more Fv, scFv or Fab fragments). Engineered antibodies with two, three or more (e.g. four) functional antigen binding sites have also been reported (see, e.g., US 2002/0004587 A1).

The term “binding (to an antigen)” denotes the binding of an antibody to its cognate antigen. Binding can be determined in an in vitro assay. In certain embodiments, binding is determined in a binding assay in which the antibody is bound to a surface and binding of the antigen to the antibody is measured by Surface Plasmon Resonance (SPR). The affinity of the binding is defined by the terms ka (rate constant for the association of the antibody from the antibody/antigen complex), kd (dissociation constant), and KD (kd/ka). Thus, binding means a specific and detectable interaction between the antibody and its cognate antigen, e.g. a binding affinity (KD) of TE-4 M or less. “Specifically binding” means a binding affinity (KD) of TE-8 M or less, in some embodiments of 1E-13 to TE-8 M, in some embodiments of 1E-13 to TE-9 M.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called and μ, respectively.

The term “cytotoxic agent” as used herein refers to a substance that inhibits or prevents a cellular function and/or causes cell death or destruction. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

The term “complement-dependent cytotoxicity (CDC)” refers to lysis of cells induced by the antibody according to the current invention in the presence of complement. CDC can be measured by the treatment of CD19 expressing human endothelial cells with an antibody according to the current invention in the presence of complement. The cells can be labeled with calcein. CDC is found if the antibody induces lysis of 20% or more of the target cells at a concentration of 30 μg/ml. Binding to the complement factor C1q can be measured in an ELISA. In such an assay, in principle, an ELISA plate is coated with concentration ranges of the antibody according to the current invention, to which purified human C1q or human serum is added. C1q binding is detected by an antibody directed against C1q followed by a peroxidase-labeled conjugate. Detection of binding (maximal binding Bmax) is measured as optical density at 405 nm (OD405) for peroxidase substrate ABTS® (2,2′-azino-di-[3-ethylbenzthiazoline-6-sulfonate (6)]).

“Effector functions” refer to those biological activities attributable to the Fc-region of an antibody, which vary with the antibody class. Examples of antibody effector functions include C1q binding and complement dependent cytotoxicity (CDC); Fc receptor binding; antibody-dependent cell-mediated cytotoxicity (ADCC); phagocytosis; down regulation of cell surface receptors (e.g. B-cell receptor); and B-cell activation.

Fc receptor binding dependent effector functions can be mediated by the interaction of the Fc-region of an antibody with Fc receptors (FcRs), which are specialized cell surface receptors on hematopoietic cells. Fc receptors belong to the immunoglobulin superfamily, and have been shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC) (see e.g. Van de Winkel, J. G. and Anderson, C. L., J. Leukoc. Biol. 49 (1991) 511-524). FcRs are defined by their specificity for immunoglobulin isotypes: Fc receptors for IgG antibodies are referred to as FcγR. Fc receptor binding is described e.g. in Ravetch, J. V. and Kinet, J. P., Annu. Rev. Immunol. 9 (1991) 457-492; Capel, P. J., et al., Immunomethods 4 (1994) 25-34; de Haas, M., et al., J. Lab. Clin. Med. 126 (1995) 330-341; and Gessner, J. E., et al., Ann. Hematol. 76 (1998) 231-248.

Cross-linking of receptors for the Fc-region of IgG antibodies (FcγR) triggers a wide variety of effector functions including phagocytosis, antibody-dependent cellular cytotoxicity, and release of inflammatory mediators, as well as immune complex clearance and regulation of antibody production. In humans, three classes of FcγR have been characterized, which are:
FcγRI (CD64) binds monomeric IgG with high affinity and is expressed on macrophages, monocytes, neutrophils and eosinophils. Modification in the Fc-region IgG at least at one of the amino acid residues E233-G236, P238, D265, N297, A327 and P329 (numbering according to EU index of Kabat) reduce binding to FcγRI. IgG2 residues at positions 233-236, substituted into IgG1 and IgG4, reduced binding to FcγRI by 103-fold and eliminated the human monocyte response to antibody-sensitized red blood cells (Armour, K. L., et al., Eur. J. Immunol. 29 (1999) 2613-2624);
FcγRII (CD32) binds complexed IgG with medium to low affinity and is widely expressed. This receptor can be divided into two sub-types, FcγRIIA and FcγRIIB. FcγRIIA is found on many cells involved in killing (e.g. macrophages, monocytes, neutrophils) and seems able to activate the killing process. FcγRIIB seems to play a role in inhibitory processes and is found on B cells, macrophages and on mast cells and eosinophils. On B-cells, it seems to function to suppress further immunoglobulin production and isotype switching to, for example, the IgE class. On macrophages, FcγRIIB acts to inhibit phagocytosis as mediated through FcγRIIA. On eosinophils and mast cells, the B-form may help to suppress activation of these cells through IgE binding to its separate receptor. Reduced binding for FcγRIIA is found e.g. for antibodies comprising an IgG Fc-region with mutations at least at one of the amino acid residues E233-G236, P238, D265, N297, A327, P329, D270, Q295, A327, R292, and K414 (numbering according to EU index of Kabat);
FcγRIII (CD16) binds IgG with medium to low affinity and exists as two types. FcγRIIIA is found on NK cells, macrophages, eosinophils and some monocytes and T cells and mediates ADCC. FcγRIIIB is highly expressed on neutrophils. Reduced binding to FcγRIIIA is found e.g. for antibodies comprising an IgG Fc-region with mutation at least at one of the amino acid residues E233-G236, P238, D265, N297, A327, P329, D270, Q295, A327, S239, E269, E293, Y296, V303, A327, K338 and D376 (numbering according to EU index of Kabat).

Mapping of the binding sites on human IgG1 for Fc receptors, the above mentioned mutation sites and methods for measuring binding to FcγRI and FcγRIIA are described in Shields, R. L., et al. J. Biol. Chem. 276 (2001) 6591-6604.

An “effective amount” of an agent, e.g., a pharmaceutical formulation, refers to an amount effective, at dosages and for periods of time necessary, to achieve the desired therapeutic or prophylactic result.

The term “Fc receptor” as used herein refers to activation receptors characterized by the presence of a cytoplasmic ITAM sequence associated with the receptor (see e.g. Ravetch, J. V. and Bolland, S., Annu. Rev. Immunol. 19 (2001) 275-290). Such receptors are FcγRI, FcγRIIA and FcγRIIIA. The term “no binding of FcγR” denotes that at an antibody concentration of 10 μg/ml the binding of the antibody to NK cells is 10% or less of the binding found for anti-OX40L antibody LC.001 as reported in WO 2006/029879.

While IgG4 shows reduced FcR binding, antibodies of other IgG subclasses show strong binding. However Pro238, Asp265, Asp270, Asn297 (loss of Fc carbohydrate), Pro329 and 234, 235, 236 and 237 Ile253, Ser254, Lys288, Thr307, Gln311, Asn434, and His435 are residues which provide if altered also reduce FcR binding (Shields, R. L., et al. J. Biol. Chem. 276 (2001) 6591-6604; Lund, J., et al., FASEB J. 9 (1995) 115-119; Morgan, A., et al., Immunology 86 (1995) 319-324; and EP 0 307 434). In certain embodiments, the antibody according to the invention is of IgG1 or IgG2 subclass and comprises the mutation PVA236, GLPSS331, L234A/L235A or P329G/L234A/L235A. In certain embodiments, the antibody as reported herein is of IgG4 subclass and comprises the mutation L235E. In certain embodiments, the antibody according to the invention further comprises the mutation S228P.

The term “Fc-region polypeptide (of human origin)” denotes the C-terminal region of an immunoglobulin heavy chain (of human origin) that contains at least a part of the hinge region, the CH2 domain and the CH3 domain. In certain embodiments, a human IgG heavy chain Fc-region polypeptide extends from Cys226, or from Pro230, to the carboxyl-terminus of the heavy chain. In one preferred embodiment, the Fc-region polypeptide comprises the amino acid sequence of SEQ ID NO: 05 or is a variant thereof. However, the C-terminal lysine (Lys447) of an Fc-region polypeptide or a complete antibody heavy chain may be present or not.

An “Fc-region of an antibody” is a term well known to the skilled artisan and defined on the basis of papain cleavage of antibodies. In certain embodiments, the Fc-region is a human Fc-region. An “Fc-region (of human origin)” comprises two heavy chain Fc-region polypeptides (of human origin), which are covalently linked to each other via the hinge region cysteine residues forming inter-chain disulfide bonds.

The antibody according to the current invention comprise as Fc-region, in certain embodiments, an Fc-region derived from human origin. In certain embodiments, the Fc-region comprises all parts of the human constant region. The Fc-region of an antibody is directly involved in complement activation, C1q binding, C3 activation and Fc receptor binding. While the influence of an antibody on the complement system is dependent on certain conditions, binding to C1q is caused by defined binding sites in the Fc-region. Such binding sites are known in the state of the art and described e.g. by Lukas, T. J., et al., J. Immunol. 127 (1981) 2555-2560; Brunhouse, R., and Cebra, J. J., Mol. Immunol. 16 (1979) 907-917; Burton, D. R., et al., Nature 288 (1980) 338-344; Thommesen, J. E., et al., Mol. Immunol. 37 (2000) 995-1004; Idusogie, E. E., et al., J. Immunol. 164 (2000) 4178-4184; Hezareh, M., et al., J. Virol. 75 (2001) 12161-12168; Morgan, A., et al., Immunology 86 (1995) 319-324; and EP 0 307 434. Such binding sites are e.g. L234, L235, D270, N297, E318, K320, K322, P331 and P329 (numbering according to EU index of Kabat; Unless otherwise specified herein, numbering of amino acid residues in the Fc-region or constant region is according to the EU numbering system, also called the EU index, as described in Kabat, E. A. et al., Sequences of Proteins of Immunological Interest, 5th ed., Public Health Service, National Institutes of Health, Bethesda, MD (1991), NIH Publication 91-3242). Antibodies of subclass IgG1, IgG2 and IgG3 usually show complement activation, C1q binding and C3 activation, whereas IgG4 do not activate the complement system, do not bind C1q and do not activate C3.

The term “FcRn” denotes the human neonatal Fc-receptor. FcRn functions to salvage IgG from the lysosomal degradation pathway, resulting in reduced clearance and increased half-life. The FcRn is a heterodimeric protein consisting of two polypeptides: a 50 kDa class I major histocompatibility complex-like protein (α-FcRn) and a 15 kDa 02-microglobulin (02m). FcRn binds with high affinity to the CH2-CH3 portion of the Fc-region of IgG. The interaction between IgG and FcRn is strictly pH dependent and occurs in a 1:2 stoichiometry, with one IgG binding to two FcRn molecules via its two heavy chains (Huber, A. H., et al., J. Mol. Biol. 230 (1993) 1077-1083). FcRn binding occurs in the endosome at acidic pH (pH<6.5) and IgG is released at the neutral cell surface (pH of about 7.4). The pH-sensitive nature of the interaction facilitates the FcRn-mediated protection of IgGs pinocytosed into cells from intracellular degradation by binding to the receptor within the acidic environment of endosomes. FcRn then facilitates the recycling of IgG to the cell surface and subsequent release into the blood stream upon exposure of the FcRn-IgG complex to the neutral pH environment outside the cell.

The term “FcRn binding portion of an Fc-region” denotes the part of an antibody heavy chain polypeptide that extends approximately from EU position 243 to EU position 261 and approximately from EU position 275 to EU position 293 and approximately from EU position 302 to EU position 319 and approximately from EU position 336 to EU position 348 and approximately from EU position 367 to EU position 393 and EU position 408 and approximately from EU position 424 to EU position 440. In one embodiment one or more of the following amino acid residues according to the EU numbering of Kabat are altered F243, P244, P245 P, K246, P247, K248, D249, T250, L251, M252, 1253, S254, R255, T256, P257, E258, V259, T260, C261, F275, N276, W277, Y278, V279, D280, V282, E283, V284, H285, N286, A287, K288, T289, K290, P291, R292, E293, V302, V303, S304, V305, L306, T307, V308, L309, H310, Q311, D312, W313, L314, N315, G316, K317, E318, Y319, 1336, S337, K338, A339, K340, G341, Q342, P343, R344, E345, P346, Q347, V348, C367, V369, F372, Y373, P374, S375, D376, 1377, A378, V379, E380, W381, E382, S383, N384, G385, Q386, P387, E388, N389, Y391, T393, S408, S424, C425, S426, V427, M428, H429, E430, A431, L432, H433, N434, H435, Y436, T437, Q438, K439, and S440 (EU numbering).

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The term “full-length antibody” denotes an antibody having a structure substantially similar to a native antibody structure. A full-length antibody comprises i) two full-length antibody light chains each comprising a variable domain and a constant domain, and ii) two full-length antibody heavy chains each comprising a variable domain, a first constant domain, a hinge region, a second constant domain and a third constant domain. A full-length antibody may comprise further domains, such as e.g. additional scFv or a scFab or a domain-exchanged Fab conjugated to one or more of the chains of the full-length antibody, preferably to the C-terminus of one or more heavy chains.

The terms “host cell” and “host cell line” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical to a parent cell, but may contain mutations. Mutant progeny that have the same function or biological activity as screened or selected for in the originally transformed cell are included herein.

A “human consensus framework” is a framework that represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat, E. A. et al., Sequences of Proteins of Immunological Interest, 5th ed., Bethesda MD (1991), NIH Publication 91-3242, Vols. 1-3. In certain embodiments, for the VL, the subgroup is subgroup kappa I as in Kabat et al., supra. In certain embodiments, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR”, as used herein, refers to each of the regions of an antibody variable domain comprising the amino acid residue stretches which are hypervariable in sequence (“complementarity determining regions” or “CDRs”) and/or form structurally defined loops (“hypervariable loops”), and/or contain the antigen-contacting residues (“antigen contacts”). Generally, antibodies comprise six HVRs; three in the VH (H1, H2, H3), and three in the VL (L1, L2, L3).

HVRs include
(a) hypervariable loops occurring at amino acid residues 26-32 (L1), 50-52 (L2), 91-96 (L3), 26-32 (H1), 53-55 (H2), and 96-101 (H3) (Chothia, C. and Lesk, A. M., J. Mol. Biol. 196 (1987) 901-917);
(b) CDRs occurring at amino acid residues 24-34 (Li), 50-56 (L2), 89-97 (L3), 31-35b (H1), 50-65 (H2), and 95-102 (H3) (Kabat, E. A. et al., Sequences of Proteins of Immunological Interest, 5th ed. Public Health Service, National Institutes of Health, Bethesda, MD (1991), NIH Publication 91-3242.);
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

The term “derived from” denotes that an amino acid sequence is derived from a parent amino acid sequence by introducing alterations at at least one position. Thus, a derived amino acid sequence differs from the corresponding parent amino acid sequence at at least one corresponding position (numbering according to Kabat EU index for antibody Fc-regions). In certain embodiments, an amino acid sequence derived from a parent amino acid sequence differs by one to fifteen amino acid residues at corresponding positions. In certain embodiments, an amino acid sequence derived from a parent amino acid sequence differs by one to ten amino acid residues at corresponding positions. In certain embodiments, an amino acid sequence derived from a parent amino acid sequence differs by one to six amino acid residues at corresponding positions. Likewise, a derived amino acid sequence has a high amino acid sequence identity to its parent amino acid sequence. In certain embodiments, an amino acid sequence derived from a parent amino acid sequence has 80% or more amino acid sequence identity. In certain embodiments, an amino acid sequence derived from a parent amino acid sequence has 90% or more amino acid sequence identity. In certain embodiments, an amino acid sequence derived from a parent amino acid sequence has 95% or more amino acid sequence identity. In one preferred embodiment, an amino acid sequence derived from a parent amino acid sequence has 98% or more amino acid sequence identity.

The term “(human) Fc-region polypeptide” denotes an amino acid sequence that is identical to a “native” or “wild-type” (human) Fc-region polypeptide. The term “variant (human) Fc-region polypeptide” denotes an amino acid sequence, which is derived from a “native” or “wild-type” (human) Fc-region polypeptide by virtue of at least one “amino acid alteration”. A “variant (human) Fc-region” is consisting of two Fc-region polypeptides, whereby both can be variant (human) Fc-region polypeptides or one is a (human) Fc-region polypeptide and the other is a variant (human) Fc-region polypeptide.

In certain embodiments, the human Fc-region polypeptide has the amino acid sequence of a human IgG1 Fc-region polypeptide or is a variant thereof, or of a human IgG2 Fc-region polypeptide or is a variant thereof, or of a human IgG3 Fc-region polypeptide or is a variant thereof, or of a human IgG4 Fc-region polypeptide or is a variant thereof. In certain embodiments, the Fc-region polypeptide is derived from an Fc-region polypeptide of SEQ ID NO: 05 or SEQ ID NO: 17 and has at least one amino acid mutation compared to the Fc-region polypeptide of SEQ ID NO: 05 or SEQ ID NO: 17. In certain embodiments, the Fc-region polypeptide comprises/has from about one to about ten amino acid mutations. In certain embodiments, the Fc-region polypeptide comprises/has from about one to about five amino acid mutations.

In certain embodiments, the Fc-region polypeptide has the amino acid sequence of a human IgG1 Fc-region polypeptide with the PGLALA mutations and the knob-mutation (SEQ ID NO: 12), wherein optionally the C-terminal lysine residue is deleted. In certain embodiments, the Fc-region polypeptide has the amino acid sequence of a human IgG1 Fc-region with the PGLALA mutations and the knob-cys or hole-cys mutations (SEQ ID NO: 16 or SEQ ID NO: 15), wherein optionally the C-terminal lysine residue is deleted.

In certain embodiments, the Fc-region polypeptide has at least about 80% sequence homology with a human Fc-region polypeptide of SEQ ID NO: 05 or SEQ ID NO: 17. In certain embodiments, the Fc-region polypeptide has at least about 90% sequence homology with a human Fc-region polypeptide of SEQ ID NO: 05 or SEQ ID NO: 17. In certain embodiments, the Fc-region polypeptide has at least about 95% sequence homology with a human Fc-region polypeptide of SEQ ID NO: 05 or SEQ ID NO: 17. In one preferred embodiment, the Fc-region polypeptide has at least about 97.5% homology with a human Fc-region polypeptide of SEQ ID NO: 05 or SEQ ID NO: 17.

In certain embodiments, the Fc-region polypeptide has at least about 80% sequence identity with a human Fc-region polypeptide of SEQ ID NO: 05 or SEQ ID NO: 17. In certain embodiments, the Fc-region polypeptide has at least about 90% sequence identity with a human Fc-region polypeptide of SEQ ID NO: 05 or SEQ ID NO: 17. In certain embodiments, the Fc-region polypeptide has at least about 95% sequence identity with a human Fc-region polypeptide of SEQ ID NO: 05 or SEQ ID NO: 17. In one preferred embodiment, the Fc-region polypeptide has at least about 97.5% identity with a human Fc-region polypeptide of SEQ ID NO: 05 or SEQ ID NO: 17. In one preferred embodiment, the C-terminal lysine residue is deleted.

A variant Fc-region polypeptide derived from a parent (human) Fc-region polypeptide of SEQ ID NO: 05 or SEQ ID NO: 17 is further defined by the amino acid alterations that are contained compared to the parent or wild-type sequence.

Thus, for example, the term P329G denotes an Fc-region polypeptide derived from a (human) Fc-region polypeptide with the mutation of proline to glycine at amino acid position 329 relative to the human Fc-region polypeptide of SEQ ID NO: 05 or SEQ ID NO: 17 (numbering according to Kabat).

A human IgG1 Fc-region polypeptide has the following amino acid sequence:

DKTHTCPPCPAPELLGGPSVFLFPPKPKDTLMISRTPEVTCVVVDVSHEDPE VKFNWYVDGVEVHNAKTKPREEQYNSTYRVVSVLTVLHQDWLNGKEYK CKVSNKALPAPIEKTISKAKGQPREPQVYTLPPSRDELTKNQVSLTCLVKGF YPSDIAVEWESNGQPENNYKTTPPVLDSDGSFFLYSKLTVDKSRWQQGNV FSCSVMHEALHNHYTQKSLSLSPG (SEQ ID NO: 05), optionally with an additional lysine residue (K) added to the C-terminus.

A human IgG1 Fc-region derived Fc-region polypeptide with the mutations L234A, L235A (LALA mutations) has the following amino acid sequence:

DKTHTCPPCPAPEAAGGPSVFLFPPKPKDTLMISRTPEVTCVVVDVSHEDPE VKFNWYVDGVEVHNAKTKPREEQYNSTYRVVSVLTVLHQDWLNGKEYK CKVSNKALPAPIEKTISKAKGQPREPQVYTLPPSRDELTKNQVSLTCLVKGF YPSDIAVEWESNGQPENNYKTTPPVLDSDGSFFLYSKLTVDKSRWQQGNV FSCSVMHEALHNHYTQKSLSLSPG (SEQ ID NO: 06), optionally with an additional lysine residue (K) added to the C-terminus.

A human IgG1 Fc-region derived Fc-region polypeptide with Y349C, T366S, L368A and Y407V mutations (knob-cys-mutations) has the following amino acid sequence:

DKTHTCPPCPAPELLGGPSVFLFPPKPKDTLMISRTPEVTCVVVDVSHEDPE VKFNWYVDGVEVHNAKTKPREEQYNSTYRVVSVLTVLHQDWLNGKEYK CKVSNKALPAPIEKTISKAKGQPREPQVCTLPPSRDELTKNQVSLSCAVKGF YPSDIAVEWESNGQPENNYKTTPPVLDSDGSFFLVSKLTVDKSRWQQGNV FSCSVMHEALHNHYTQKSLSLSPG (SEQ ID NO: 07), optionally with an additional lysine residue (K) added to the C-terminus.

A human IgG1 Fc-region derived Fc-region polypeptide with S354C, T366W (hole-cys-mutations) mutations has the following amino acid sequence:

DKTHTCPPCPAPELLGGPSVFLFPPKPKDTLMISRTPEVTCVVVDVSHEDPE VKFNWYVDGVEVHNAKTKPREEQYNSTYRVVSVLTVLHQDWLNGKEYK CKVSNKALPAPIEKTISKAKGQPREPQVYTLPPCRDELTKNQVSLWCLVKG FYPSDIAVEWESNGQPENNYKTTPPVLDSDGSFFLYSKLTVDKSRWQQGN VFSCSVMHEALHNHYTQKSLSLSPG (SEQ ID NO: 08), optionally with an additional lysine residue (K) added to the C-terminus.

A human IgG1 Fc-region derived Fc-region polypeptide with L234A, L235A mutations and Y349C, T366S, L368A, Y407V mutations has the following amino acid sequence:

DKTHTCPPCPAPEAAGGPSVFLFPPKPKDTLMISRTPEVTCVVVDVSHEDPE VKFNWYVDGVEVHNAKTKPREEQYNSTYRVVSVLTVLHQDWLNGKEYK CKVSNKALPAPIEKTISKAKGQPREPQVCTLPPSRDELTKNQVSLSCAVKGF YPSDIAVEWESNGQPENNYKTTPPVLDSDGSFFLVSKLTVDKSRWQQGNV FSCSVMHEALHNHYTQKSLSLSPG (SEQ ID NO: 09), optionally with an additional lysine residue (K) added to the C-terminus.

A human IgG1 Fc-region derived Fc-region polypeptide with a L234A, L235A and S354C, T366W mutations has the following amino acid sequence:

DKTHTCPPCPAPEAAGGPSVFLFPPKPKDTLMISRTPEVTCVVVDVSHEDPE VKFNWYVDGVEVHNAKTKPREEQYNSTYRVVSVLTVLHQDWLNGKEYK CKVSNKALPAPIEKTISKAKGQPREPQVYTLPPCRDELTKNQVSLWCLVKG FYPSDIAVEWESNGQPENNYKTTPPVLDSDGSFFLYSKLTVDKSRWQQGN VFSCSVMHEALHNHYTQKSLSLSPG (SEQ ID NO: 10), optionally with an additional lysine residue (K) added to the C-terminus.

A human IgG1 Fc-region derived Fc-region polypeptide with a P329G mutation (PG mutation) has the following amino acid sequence:

DKTHTCPPCPAPELLGGPSVFLFPPKPKDTLMISRTPEVTCVVVDVSHEDPE VKFNWYVDGVEVHNAKTKPREEQYNSTYRVVSVLTVLHQDWLNGKEYK CKVSNKALGAPIEKTISKAKGQPREPQVYTLPPSRDELTKNQVSLTCLVKG FYPSDIAVEWESNGQPENNYKTTPPVLDSDGSFFLYSKLTVDKSRWQQGN VFSCSVMHEALHNHYTQKSLSLSPG (SEQ ID NO: 11), optionally with an additional lysine residue (K) added to the C-terminus.

A human IgG1 Fc-region derived Fc-region polypeptide with L234A, L235A mutations and P329G mutation (LALAPG mutations) has the following amino acid sequence:

DKTHTCPPCPAPEAAGGPSVFLFPPKPKDTLMISRTPEVTCVVVDVSHEDPE VKFNWYVDGVEVHNAKTKPREEQYNSTYRVVSVLTVLHQDWLNGKEYK CKVSNKALGAPIEKTISKAKGQPREPQVYTLPPSRDELTKNQVSLTCLVKG FYPSDIAVEWESNGQPENNYKTTPPVLDSDGSFFLYSKLTVDKSRWQQGN VFSCSVMHEALHNHYTQKSLSLSPG (SEQ ID NO: 12), optionally with an additional lysine residue (K) added to the C-terminus.

A human IgG1 Fc-region derived Fc-region polypeptide with a P329G mutation and Y349C, T366S, L368A, Y407V mutations has the following amino acid sequence:

DKTHTCPPCPAPELLGGPSVFLFPPKPKDTLMISRTPEVTCVVVDVSHEDPE VKFNWYVDGVEVHNAKTKPREEQYNSTYRVVSVLTVLHQDWLNGKEYK CKVSNKALGAPIEKTISKAKGQPREPQVCTLPPSRDELTKNQVSLSCAVKG FYPSDIAVEWESNGQPENNYKTTPPVLDSDGSFFLVSKLTVDKSRWQQGN VFSCSVMHEALHNHYTQKSLSLSPG (SEQ ID NO: 13), optionally with an additional lysine residue (K) added to the C-terminus.

A human IgG1 Fc-region derived Fc-region polypeptide with a P329G mutation and S354C, T366W mutation has the following amino acid sequence:

DKTHTCPPCPAPELLGGPSVFLFPPKPKDTLMISRTPEVTCVVVDVSHEDPE VKFNWYVDGVEVHNAKTKPREEQYNSTYRVVSVLTVLHQDWLNGKEYK CKVSNKALGAPIEKTISKAKGQPREPQVYTLPPCRDELTKNQVSLWCLVKG FYPSDIAVEWESNGQPENNYKTTPPVLDSDGSFFLYSKLTVDKSRWQQGN VFSCSVMHEALHNHYTQKSLSLSPG (SEQ ID NO: 14), optionally with an additional lysine residue (K) added to the C-terminus.

A human IgG1 Fc-region derived Fc-region polypeptide with L234A, L235A, P329G and Y349C, T366S, L368A, Y407V mutations has the following amino acid sequence:

DKTHTCPPCPAPEAAGGPSVFLFPPKPKDTLMISRTPEVTCVVVDVSHEDPE VKFNWYVDGVEVHNAKTKPREEQYNSTYRVVSVLTVLHQDWLNGKEYK CKVSNKALGAPIEKTISKAKGQPREPQVCTLPPSRDELTKNQVSLSCAVKG FYPSDIAVEWESNGQPENNYKTTPPVLDSDGSFFLVSKLTVDKSRWQQGN VFSCSVMHEALHNHYTQKSLSLSPG (SEQ ID NO: 15), optionally with an additional lysine residue (K) added to the C-terminus.

A human IgG1 Fc-region derived Fc-region polypeptide with L234A, L235A, P329G mutations and S354C, T366W mutations has the following amino acid sequence:

DKTHTCPPCPAPEAAGGPSVFLFPPKPKDTLMISRTPEVTCVVVDVSHEDPE VKFNWYVDGVEVHNAKTKPREEQYNSTYRVVSVLTVLHQDWLNGKEYK CKVSNKALGAPIEKTISKAKGQPREPQVYTLPPCRDELTKNQVSLWCLVKG FYPSDIAVEWESNGQPENNYKTTPPVLDSDGSFFLYSKLTVDKSRWQQGN VFSCSVMHEALHNHYTQKSLSLSPG (SEQ ID NO: 16), optionally with an additional lysine residue (K) added to the C-terminus.

A human IgG4 Fc-region polypeptide has the following amino acid sequence:

(SEQ ID NO: 17)
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

A human IgG4 Fc-region derived Fc-region polypeptide with S228P and L235E mutations has the following amino acid sequence:

(SEQ ID NO: 18)
ESKYGPPCPPCPAPEFEGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

A human IgG4 Fc-region derived Fc-region polypeptide with S228P, L235E mutations and P329G mutation has the following amino acid sequence:

(SEQ ID NO: 19)
ESKYGPPCPPCPAPEFEGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLGSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

A human IgG4 Fc-region derived Fc-region polypeptide with S354C, T366W mutations has the following amino acid sequence:

(SEQ ID NO: 20)
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPCQEEMTKNQVSL
WCLVKGFYPSDIAVEWESNGQPENNYKTTPPVLDSDGSFFLYSRLTVDK
SRWQEGNVFSCSVMHEALHNHYTQKSLSLSLGK.

A human IgG4 Fc-region derived Fc-region polypeptide with Y349C, T366S, L368A, Y407V mutations has the following amino acid sequence:

(SEQ ID NO: 21)
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVCTLPPSQEEMTKNQVSL
SCAVKGFYPSDIAVEWESNGQPENNYKTTPPVLDSDGSFFLVSRLTVDK
SRWQEGNVFSCSVMHEALHNHYTQKSLSLSLGK.

A human IgG4 Fc-region derived Fc-region polypeptide with a S228P, L235E and S354C, T366W mutations has the following amino acid sequence:

(SEQ ID NO: 22)
ESKYGPPCPPCPAPEFEGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVYTLPPCQEEMTKNQVSL
WCLVKGFYPSDIAVEWESNGQPENNYKTTPPVLDSDGSFFLYSRLTVDK
SRWQEGNVFSCSVMHEALHNHYTQKSLSLSLGK.

A human IgG4 Fc-region derived Fc-region polypeptide with a S228P, L235E and Y349C, T366S, L368A, Y407V mutations has the following amino acid sequence:

(SEQ ID NO: 23)
ESKYGPPCPPCPAPEFEGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLPSSIEKTISKAKGQPREPQVCTLPPSQEEMTKNQVSL
SCAVKGFYPSDIAVEWESNGQPENNYKTTPPVLDSDGSFFLVSRLTVDK
SRWQEGNVFSCSVMHEALHNHYTQKSLSLSLGK.

A human IgG4 Fc-region derived Fc-region polypeptide with a P329G mutation has the following amino acid sequence:

(SEQ ID NO: 24)
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLGSSIEKTISKAKGQPREPQVYTLPPSQEEMTKNQVSL
TCLVKGFYPSDIAVEWESNGQPENNYKTTPPVLDSDGSFFLYSRLTVDK
SRWQEGNVFSCSVMHEALHNHYTQKSLSLSLGK.

A human IgG4 Fc-region derived Fc-region polypeptide with a P329G and Y349C, T366S, L368A, Y407V mutations has the following amino acid sequence:

(SEQ ID NO: 25)
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLGSSIEKTISKAKGQPREPQVCTLPPSQEEMTKNQVSL
SCAVKGFYPSDIAVEWESNGQPENNYKTTPPVLDSDGSFFLVSRLTVDK
SRWQEGNVFSCSVMHEALHNHYTQKSLSLSLGK.

A human IgG4 Fc-region derived Fc-region polypeptide with a P329G and S354C, T366W mutations has the following amino acid sequence:

(SEQ ID NO: 26)
ESKYGPPCPSCPAPEFLGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLGSSIEKTISKAKGQPREPQVYTLPPCQEEMTKNQVSL
WCLVKGFYPSDIAVEWESNGQPENNYKTTPPVLDSDGSFFLYSRLTVDK
SRWQEGNVFSCSVMHEALHNHYTQKSLSLSLGK.

A human IgG4 Fc-region derived Fc-region polypeptide with a S228P, L235E, P329G and Y349C, T366S, L368A, Y407V mutations has the following amino acid sequence:

(SEQ ID NO: 27)
ESKYGPPCPPCPAPEFEGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLGSSIEKTISKAKGQPREPQVCTLPPSQEEMTKNQVSL
SCAVKGFYPSDIAVEWESNGQPENNYKTTPPVLDSDGSFFLVSRLTVDK
SRWQEGNVFSCSVMHEALHNHYTQKSLSLSLGK

A human IgG4 Fc-region derived Fc-region polypeptide with a S228P, L235E, P329G and S354C, T366W mutations has the following amino acid sequence:

(SEQ ID NO: 28)
ESKYGPPCPPCPAPEFEGGPSVFLFPPKPKDTLMISRTPEVTCVVVDVS
QEDPEVQFNWYVDGVEVHNAKTKPREEQFNSTYRVVSVLTVLHQDWLNG
KEYKCKVSNKGLGSSIEKTISKAKGQPREPQVYTLPPCQEEMTKNQVSL
WCLVKGFYPSDIAVEWESNGQPENNYKTTPPVLDSDGSFFLYSRLTVDK
SRWQEGNVFSCSVMHEALHNHYTQKSLSLSLGK

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., the CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “isolated” antibody is one that has been separated from a component of its natural environment. In certain embodiments, an antibody is purified to greater than 95% or 99% purity as determined by, for example, electrophoretic (e.g., SDS-PAGE, isoelectric focusing (IEF), capillary electrophoresis) or chromatographic (e.g., size exclusion chromatography or ion exchange or reverse phase HPLC) analytical methods. For review of methods for assessment of antibody purity, see, e.g., Flatman, S. et al., J. Chrom. B 848 (2007) 79-87.

An “isolated” nucleic acid refers to a nucleic acid molecule that has been separated from a component of its natural environment.

An “isolated nucleic acid encoding an anti-human A-beta protein antibody” denotes to one or more nucleic acid molecules encoding the antibody heavy and light chains (or fragments thereof), including such nucleic acid molecule(s) in a single plasmid or separate plasmids.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g. cows, sheep, cats, dogs, and horses), primates (e.g., humans and non-human primates such as monkeys), rabbits, and rodents (e.g., mice and rats). In certain embodiments, the individual or subject is a human.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 Daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S.

Copyright Registration No. TXU510087. The ALIGN-2 program is publicly available from Genentech, Inc., South San Francisco, California, or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no components which are unacceptably toxic to a subject to which the formulation would be administered.

A “pharmaceutically acceptable carrier” refers to an ingredient in a pharmaceutical formulation, other than an active ingredient, which is non-toxic to a subject., A pharmaceutically acceptable carrier includes, but is not limited to, a buffer, excipient, stabilizer, or preservative.

The term “plasmid”, as used herein, refers to a nucleic acid molecule capable of propagating another nucleic acid that they comprise. The term includes the plasmid as a self-replicating nucleic acid structure as well as the plasmid incorporated into the genome of a host cell into which it has been introduced. Certain plasmids are capable of directing the expression of nucleic acids that they comprise. Such plasmids are referred to herein as “expression plasmid”.

The term “recombinant antibody”, as used herein, denotes all antibodies (chimeric, humanized and human) that are prepared, expressed, created or isolated by recombinant means. This includes antibodies isolated from a host cell such as a NSO, HEK, BHK or CHO cell or from an animal (e.g. a mouse) that is transgenic for human immunoglobulin genes or antibodies expressed using a recombinant expression plasmid transfected into a host cell. The amino acid sequences of the VH and VL regions of the recombinant antibodies are sequences that, while derived from and related to human germline VH and VL sequences, may not naturally exist within the human antibody germline repertoire in vivo.

As used herein, “treatment” (and grammatical variations thereof such as “treat” or “treating”) refers to a clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, decreasing the rate of disease progression, amelioration or palliation of the disease state, and remission or improved prognosis. In certain embodiments, an antibody according to the current invention is used to delay development of a disease or to slow the progression of a disease.

The term “valent” as used within the current application denotes the presence of a specified number of binding sites in a (antibody) molecule. As such, the terms “bivalent”, “trivalent” and “tetravalent” denote the presence of two binding sites, three binding sites, and four binding sites, respectively, in a (antibody) molecule.

The term “variable region” or “variable domain” refers to the domain of an antibody heavy or light chain that is involved in binding of the antibody to its antigen. The variable domains of the heavy chain and light chain (VH and VL, respectively) of an antibody generally have similar structures, with each domain comprising four framework regions (FRs) and three hypervariable regions (HVRs) (see, e.g., Kindt, T. J. et al. Kuby Immunology, 6th ed., W.H. Freeman and Co., N.Y. (2007), page 91). A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano, S. et al., J. Immunol. 150 (1993) 880-887; Clackson, T. et al., Nature 352 (1991) 624-628). A (cognate) pair of an antibody heavy chain variable domain and an antibody light chain variable domain form a binding site.

The term “variant” denotes molecules that have an amino acid sequence that differs from the amino acid sequence of a respective parent molecule. Typically, such molecules have one or more alterations (mutations), insertions, or deletions. In certain embodiments, the antibody according to the current invention comprises at least a portion of an Fc-region, which is not naturally occurring. Such molecules have less than 100% sequence identity with the parent antibody. In certain embodiments, the variant antibody has an amino acid sequence that has from about 75% to less than 100% amino acid sequence identity with the amino acid sequence of the parent antibody, especially from about 80% to less than 100%, especially from about 85% to less than 100%, especially from about 90% to less than 100%, and especially from about 95% to less than 100%. In one preferred embodiment, the parent antibody and the variant antibody differ by one (a single), two, three, five, seven or ten amino acid residue(s).

### The Amyloid Hypothesis

Amyloid plaques form when pieces of protein, called beta-amyloid, aggregate. The beta-amyloid is produced when a much larger protein referred to as the amyloid precursor protein (APP) is broken down. APP is composed of 771 amino acids and is cleaved by two enzymes to produce beta-amyloid. The large protein is first cut by beta-secretase and then by gamma-secretase, producing beta-amyloid pieces that may be made up of 38, 40 or 42 amino acids. The beta-amyloid composed of 42 amino acids is chemically “stickier” than the other lengths and therefore is more likely to form plaques. Research has shown that three genetic abnormalities that are associated with early stage Alzheimer's disease each change the function of gamma-secretase in a way that leads to an increased production of Aβ42.

How beta-amyloid causes toxic damage to nerve cells is not quite clear, but some research suggests that it may split into fragments and release free radicals, which then attack neurons. Another theory is that the beta-amyloid forms tiny holes in neuronal membranes, which leads to an unregulated influx of calcium that can cause neuronal death. Regardless of the exact pathological process through which beta-amyloid causes neuronal damage, the result is that neurons die.

Plaques are formed that are made up of a mixture of these degenerating neurons and the beta-amyloid aggregates. These plaques cannot be broken down and removed by the body, so they gradually accumulate in the brain. The accumulation of this amyloid leads to amyloidosis, which is thought to contribute to a number of neurodegenerative diseases.

Amyloid plaques form one of the two defining features of Alzheimer's disease, the other being neurofibrillary tangles. Beta-amyloid is also thought to be responsible for the formation of these tangles, which again damage neurons and cause the symptoms of dementia. Technically, a person may present with all of the characteristics of Alzheimer's disease but if a brain biopsy or positron emission tomography does not reveal the presence of amyloid plaques or neurofibrillary tangles, a diagnosis of Alzheimer's disease will not be made.

### DESCRIPTION OF EMBODIMENTS OF THE INVENTION

Gantenerumab is a fully human IgG1 antibody, which binds with sub-nanomolar affinity to a conformational epitope of Aβ consisting of both N terminal and central amino acids. It prefers binding to the fibrillary forms of the protein. The therapeutic rationale for this antibody is that it acts centrally to disassemble and degrade amyloid plaques by recruiting microglia and activating phagocytosis. It prevents new plaque formation. Gantenerumab preferentially interacts with aggregated brain Aβ, both parenchymal and vascular. The antibody elicits phagocytosis of human Aβ deposits in AD brain slices co-cultured with human macrophages. It also neutralizes oligomeric Aβ42-mediated inhibitory effects on long-term potentiation in rat brains. In APP/PS1 transgenic mice, gantenerumab binds to cerebral Aβ, reduces small plaques by recruiting microglia, and prevents new plaque formation. Gantenerumab does not alter systemic levels of Aβ, which suggests that clearance of soluble Aβ is undisturbed. In the phase 3 multi-center, randomized, double-blind, placebo controlled trial SCarlet RoAD gantenerumab showed target engagement, resulting in clearance of plaques, and reduced levels of phosphorylated tau in the spinal cord fluid (see, e.g., Sumner, I. L., et al., Front. Neurosci. 12 (2018) article 254; https://www.alzforum.org/therapeutics/gantenerumab).

Gantenerumab has two groups of glycosylation sites: the first group encompasses the glycosylation sites in each of the Fabs in the heavy chain CDR2s, the second group encompasses the glycosylation sites in each of the Fc-regions at Asn 297 (numbering according to Kabat).

The glycosylation pattern of the glycosylation sites, i.e. the glyco-occupation, is different between both groups of glycosylation sites. The glyco-occupation of each of the glycosylation sites in the Fc-region is homogeneous and comparable to each other as well as to other recombinantly produced antibodies. In contrast thereto, the glyco-occupation of the glycosylation site in the Fabs is diverging, i.e. either both glycosylation sites are glycosylated, or only one of these glycosylation sites is glycosylated, or none of these glycosylation sites is glycosylated. Thus, recombinantly produced gantenerumab is obtained as a mixture of three different glycosylation isoforms from the producing cells. As the glyco-occupation and glycosylation pattern influence pharmacokinetic properties as well as plaque binding, the non-glycosylated isoform needs to be removed. The different glycoforms can be distinguished and separated using chromatography, such as, e.g. hydrophobic interaction chromatography (HIC).

It has now been found by the current inventors that by introducing a specific mutation in the heavy chain CDR2 and by shortening the heavy chain CDR3 the properties of gantenerumab have been improved. Without being bound by this theory, it is assumed that the introduced modifications resulted in the improved properties by reducing glyco-occupation heterogeneity in the Fab and by removing a de-amidation hotspot and thereby reducing aggregation tendency of the parent antibody gantenerumab.

Additionally, it has been found by the current inventors that the light chain of the parent antibody gantenerumab does not require modification in order to retain binding affinity and specificity when combined with the modified heavy chain according to the current invention.

Thus, aspects of the current invention are antibodies against the human A-beta protein (anti-A-beta protein antibodies), methods for their production, pharmaceutical compositions containing these antibodies, and uses thereof.

The antibodies according to the current invention are variants of the anti-A-beta antibody gantenerumab. They have improved technical and biological properties compared to their parent antibody. The improvements encompass, amongst other things, an improved production titer, an improved production yield, an improved glycosylation homogeneity, a reduced aggregation tendency and an improved process robustness.

The anti-A-beta protein antibody according to the current invention has substantial and up to 100% glyco-occupation of the glycosylation site in the heavy chain CDR2s.

The anti-A-beta protein antibodies according to the current invention have, amongst other things, improved properties in terms of target binding, developability properties, IHC/plaque binding and PK behavior.

The current invention is based, at least in part, on the finding, that complete removal of the glycosylation site in the Fab of gantenerumab resulted in a reduction of plaque binding or increased clearance, respectively. Thus, a modification resulting in reduced or even eliminated glycosylation of gantenerumab's Fab glycosylation sites is disadvantageous and needs to be prevented.

Four different variants of gantenerumab with modified Fab glyco-occupation have been generated:
two variants with modified heavy chain (numbering according to Kabat):
   complete Fab glycosylation by introducing a heavy chain S53T mutation (mAb 675);
   complete removal of Fab glycosylation by introducing a heavy chain N52Q mutation (mAb 663);
two variants with modified heavy and light chains (numbering according to Kabat):
   complete Fab glycosylation by introducing a heavy chain S53T mutation and compensation of the heavy chain modification by introducing a light chain M95H mutation (mAb 651);
   complete removal of Fab glycosylation by introducing a heavy chain N52Q mutation and compensation of the heavy chain modification by introducing a light chain M95H mutation (mAb 638).

The four variants of gantenerumab are either fully Fab glycosylated or completely without Fab glycosylation. Except for mAb 651, all variants have the same in vitro binding affinity to/for human A-beta protein.

| | | light | | Fab | A-beta |
|---|---|---|---|---|---|
| | heavy | chain | heavy | glycosylation | protein |
| | chain | variable | chain | (CE-SDS) | binding |
| antibody | CDR3 | domain | CDR2 | [%] | (EC50) |
| gantenerumab | wt | wt | wt: NAS | 30-80 | 0.2 nM |
| mAb 675 | shortened | wt | NAT | 100 | 0.2 nM |
| mAb 663 | shortened | wt | QAS | 0 | 0.2 nM |
| mAb 651 | shortened | M95H | NAT | 100 | 4 nM |
| mAb 638 | shortened | M95H | QAS | 0 | 0.2 nM |

The different glycosylation levels have been confirmed by hydrophilic interaction chromatography (HIC) as shown in FIGS. 1 to 3.

FIG. 1 shows the partial glycosylation of the parent antibody gantenerumab with the sequence NAS in the heavy chain CDRs (two peaks at an aligned time of about 22-24 sec.).

FIG. 2 shows the complete non-glycosylation of the variant antibody mAb 663 with the sequence QAS in the heavy chain CDRs (one peaks at an aligned time of about 22 sec.).

FIG. 3 shows the complete glycosylation of the variant antibody mAb 675 with the sequence QAS in the heavy chain CDRs (one peaks at an aligned time of about 23 sec.).

The antibodies according to the current invention as well as gantenerumab have been produced in small scale and the by-product distribution has been analyzed after a first purification step using a protein A affinity chromatography and after the second purification step using a preparative size-exclusion chromatography. The results are presented in the following Table.

| | main | | | | | |
|---|---|---|---|---|---|---|
| | product | | | | | |
| | after | | | | | |
| | preparative | | | | | |
| | protein A | | | end | | |
| | purification | by-products | | product | by-products | |
| | monomer | SEC [%] | | by SEC | SEC [%] | |
| antibody | by SEC [%] | HMW | LMW | [%] | HMW | LMW |
| gantenerumab | 100 | 0 | 0 | n.d. | n.d. | n.d. |
| mAb 675 | 95.4 | 3.3 | 1.3 | n.d. | n.d. | n.d. |
| mAb 663 | 89.9 | 0 | 10.1 | 96.8 | 0 | 3.2 |
| mAb 651 | 99.6 | 0.4 | 0 | n.d. | n.d. | n.d. |
| mAb 638 | 100 | 0 | 0 | n.d. | n.d. | n.d. |

n.d.: not determined - no SEC performed as sufficient purity already after protein A chromatography

The four variants have further been characterized with respect to their binding properties and specificities. The qualitative results are shown in the following table:

| antibody | mAb | mAb | mAb | mAb | |
|---|---|---|---|---|---|
| property | 675 | 663 | 651 | 638 | gantenerumab |
| A-beta binding | +++ | +++ | + | +++ | +++ |
| (ELISA) | | | | | |
| in vitro plaque | ++++/− | ++/+++ | +/− | ++/+++ | ++++/++ |
| decoration/ | | | | | |
| background | | | | | |
| in vivo plaque | +++ | ++ | + | ++ | ++ |
| decoration | | | | | |
| plasma | ok | accelerated | ok | ok | ok |
| clearance | | | | | |
| (rat) | | | | | |
| developability | ok | (ok) | n.d. | (ok) | (ok) |

The individual properties are discussed in the following in more detail.

### In Vitro A-Beta Plaque Decoration

The parent antibody gantenerumab shows strong specific plaque binding and some background, non-specific binding, especially at a concentration of 1 μg/ml (FIG. 4).

MAb 675 (“142”) shows strong specific plaque binding and no detectable background, non-specific binding (FIG. 5).

MAb 663 (“143”) shows low specific plaque binding and some background, non-specific binding, especially at a concentration of 1 μg/ml (FIG. 6).

MAb 651 (“144”) shows almost no specific plaque binding as well as no background binding and non-specific binding, even at a concentration of 1 μg/ml (FIG. 7).

MAb 638 shows low specific plaque binding and some background, non-specific binding, especially at a concentration of 1 μg/ml.

Thus, mAb 675 unexpectedly showed equal specific plaque binding when compared to gantenerumab but much less background, non-specific binding. Thus, it has been found that fully glycosylated glycosylation sites in the Fab reduce background, non-specific binding in vitro at a concentration of 1 μg/mL.

The staining results are summarized in the following Table in a qualitative form.

| | antibody | specific | background, |
|---|---|---|---|
| | concentration | plaque | non-specific |
| antibody | [(mg/ml] | staining | staining |
| gantenerumab | 0.1 | ++++ | + |
| | 1.0 | ++++ | ++ |
| mAb 675 | 0.1 | ++++ | − |
| | 1.0 | ++++ | − |
| mAb 663 | 0.1 | + | + |
| | 1.0 | ++ | +++ |
| mAb 651 | 0.1 | − | − |
| | 1.0 | + | − |
| mAb 638 | 0.1 | + | + |
| | 1.0 | ++ | +++ |

### In Vivo A-Beta Plaque Decoration

To male APP/PS2 mice (9 months of age, n=3 per group) 20 mg/kg each of gantenerumab and the four variants has been administered. Seven days post injection antibody distribution in brains slices was determined (detection antibody: goat anti-human IgG antibody conjugated to Alexa 555 (Invitrogen (#A21433); co-staining of A-beta protein: Amylo Glo, Biosensis cat #TR-300-AG; picture settings: 30× magnification). The results are shown in FIGS. 8 and 9.

Thus, mAb 675 (“142” in FIGS. 8 and 9) unexpectedly showed improved in vivo plaque binding when compared to gantenerumab. Thus, it has been found that fully glycosylated glycosylation sites in the Fab increase in vivo A-beta plaque binding.

### Pharmacokinetics

Pharmacokinetics of the four gantenerumab variants were determined in rat and mouse.

It has been found that mAb 663 without Fab glycosylation and without modification in the light chain variable domain showed an increased plasma clearance rate in rats.

All other variants show similar plasma clearance rates as gantenerumab.

The results are shown in FIG. 10.

Unexpectedly, mAb 638, mAb 675 and mAb 651 have a lower clearance rate than gantenerumab (historical data). It has to be pointed out that the difference in the plasma clearance rate does not explain the differences in plaque decoration for mAb 638.

The single-dose pharmacokinetic (SDPK) properties of the gantenerumab variants has further been evaluated using a combination of FcRn and heparin affinity chromatography.

The combination of FcRn affinity chromatography and heparin affinity chromatography allows defining FcRn and heparin affinity chromatography column retention time thresholds and thereby a two-dimensional retention time region, wherein antibodies with slow clearance, i.e. long systemic circulation half-live, can be identified. Thus, this combination allows amongst other things for the selection of antibodies with long systemic circulation half-live.

For the characterization, the retention times on an FcRn affinity chromatography column and on a heparin affinity chromatography column are normalized based on the retention times of reference antibodies on the respective columns. Thereby a relative retention time region comprising predominantly antibodies with slow clearance is defined. In more detail, this region is defined by a relative retention time on the FcRn affinity chromatography column of less than 1.78 (with an oxidized (H₂O₂-treated) anti-Her3 antibody preparation as reference antibody) and by a relative retention time on the heparin affinity chromatography column of less than 0.87 (with an anti-pTau antibody as reference antibody). For more details on the method, see WO 2018/197533.

| | relative retention on heparin | relative retention on FcRn |
|---|---|---|
| antibody | chromatography column | chromatography column |
| mAb 675 | 0.74 | 0.169 |
| mAb 663 | 1.05 | 1.586 |
| mAb 638 | 1.00 | 3 peaks |

### Thermal Stability

The thermal stability of mAb 675, mAb 663 and mAb 638 has been determined by dynamic light scattering. The aggregation temperature (T_{agg}) and melting temperature (Tₘ) have been determined by application of a heat ramp. Unexpectedly, all three tested variants show improved values compared to the parent gantenerumab.

| | mAb 675 | mAb 663 | mAb 638 | gantenerumab |
|---|---|---|---|---|
| Thermal stability | 69° C. | 72.5° C. | 73° C. | 64-66° C. |
| (DLS T_{agg}/DLS Tₘ) | | | | |

### Stability Assessment

MAb 675, mAb 663 and mAb 638 have been subjected to thermal stress by incubation at elevated temperature in different buffer systems.

It has been found that mAb 638 shows a substantial monomer loss by incubation in phosphate buffered saline solution.

However and unexpectedly, all three gantenerumab variants showed an improved stability with respect to target binding compared to the parent gantenerumab.

| | | mAb | mAb | mAb | |
|---|---|---|---|---|---|
| parameter | | 675 | 663 | 638 | gantenerumab |
| CE-SDS [%] | reference | 98 | 97 | 98 | 98 |
| (Caliper, | condition 1 | 96 | 93 | 96 | 97 |
| non-reducing) | condition 2 | 89 | 87 | 81 | 92 |
| SEC | reference | 97 | 100 | 97 | 99 |
| monomer | condition 1 | 96 | 97 | 95 | 97 |
| [%] | condition 2 | 96 | 97 | 92 | 97 |
| Target | reference | 100 | 100 | 100 | 100 |
| binding ± | condition 1 | 94 | 113 | 93 | 93 |
| stress, by | condition 2 | 85 | 102 | 85 | 70 |
| BIAcore | | | | | |

reference: pH 6.0, 20 mM His/His*HCl, 140 mM NaCl; unstressed (frozen −80° C.)
condition 1: pH 6.0, 20 mM His/His*HCl, 140 mM NaCl; stressed at 40° C. for 14 days
condition 2: pH 7.4, PBS; stressed at 37° C. for 14 days

In view of all data (100% homogeneous Fab glyco-occupation, de-amidation site removed, wild-type light chain) mAb 675 was identified as the most advantageous gantenerumab variant with improved properties.

Exemplary Anti-Human a-Beta Protein Antibodies According to the Current Invention

Thus, the current invention encompasses at least the following embodiments:

In one aspect, the invention provides antibodies that bind to human A-beta protein.

In one aspect, the invention provides isolated antibodies that bind to human A-beta protein.

In one aspect, the invention provides antibodies that specifically bind to human A-beta protein.

In certain embodiments of all aspects and embodiment of the current invention, the anti-A-beta antibody according to the current invention has one or more of the following properties
specifically binds to a human A-beta protein of SEQ ID NO: 45;
has a glycosylation site in the heavy chain CDR2 that has a glyco-occupancy of at least 95% as determined by CE-SDS;
is free of species with any non-glycosylated glycosylation site;
has an EC50 value for the human A-beta protein of SEQ ID NO: 45 of 0.5 nM or less;
is binding to A-beta plaques in vitro and not binding to non-A-beta protein molecules in brain samples at a staining concentration of up to and including 1 μg/mL;
is binding to A-beta plaques in vivo more strongly than gantenerumab;
has a relative occupancy of plaques in vivo in the cortex and hippocampus of more than 0.2 determine in a one-way Anova;
has a thermal stability (DLS T_{agg}/DLS Tₘ) of more than 68° C.

In one aspect, the invention provides an anti-A-beta protein antibody comprising at least one, at least two, at least three, at least four, at least five, or all six CDRs selected from
(1)
(a) CDR-H1 comprising the amino acid sequence of SEQ ID NO: 85;
(b) CDR-H2 comprising the amino acid sequence of SEQ ID NO: 86;
(c) CDR-H3 comprising the amino acid sequence of SEQ ID NO: 87;
(d) CDR-L1 comprising the amino acid sequence of SEQ ID NO: 81;
(e) CDR-L2 comprising the amino acid sequence of SEQ ID NO: 82; and (f) CDR-L3 comprising the amino acid sequence of SEQ ID NO: 83;
or
(2)
(a) CDR-H1 comprising the amino acid sequence of SEQ ID NO: 85;
(b) CDR-H2 comprising the amino acid sequence of SEQ ID NO: 89;
(c) CDR-H3 comprising the amino acid sequence of SEQ ID NO: 87;
(d) CDR-L1 comprising the amino acid sequence of SEQ ID NO: 81;
(e) CDR-L2 comprising the amino acid sequence of SEQ ID NO: 82; and
(f) CDR-L3 comprising the amino acid sequence of SEQ ID NO: 83;
or
(3)
(a) CDR-H1 comprising the amino acid sequence of SEQ ID NO: 85;
(b) CDR-H2 comprising the amino acid sequence of SEQ ID NO: 86;
(c) CDR-H3 comprising the amino acid sequence of SEQ ID NO: 87;
(d) CDR-L1 comprising the amino acid sequence of SEQ ID NO: 81;
(e) CDR-L2 comprising the amino acid sequence of SEQ ID NO: 82; and
(f) CDR-L3 comprising the amino acid sequence of SEQ ID NO: 91;
or
(4)
(a) CDR-H1 comprising the amino acid sequence of SEQ ID NO: 85;
(b) CDR-H2 comprising the amino acid sequence of SEQ ID NO: 89;
(c) CDR-H3 comprising the amino acid sequence of SEQ ID NO: 87;
(d) CDR-L1 comprising the amino acid sequence of SEQ ID NO: 81;
(e) CDR-L2 comprising the amino acid sequence of SEQ ID NO: 82; and
(f) CDR-L3 comprising the amino acid sequence of SEQ ID NO: 91.

In one aspect, the invention provides an antibody comprising at least one, at least two, or all three VH CDR sequences selected from
(1)
(a) CDR-H1 comprising the amino acid sequence of SEQ ID NO: 85;
(b) CDR-H2 comprising the amino acid sequence of SEQ ID NO: 86; and
(c) CDR-H3 comprising the amino acid sequence of SEQ ID NO: 87;
or
(2)
(a) CDR-H1 comprising the amino acid sequence of SEQ ID NO: 85;
(b) CDR-H2 comprising the amino acid sequence of SEQ ID NO: 89; and
(c) CDR-H3 comprising the amino acid sequence of SEQ ID NO: 87;
or
(3)
(a) CDR-H1 comprising the amino acid sequence of SEQ ID NO: 85;
(b) CDR-H2 comprising the amino acid sequence of SEQ ID NO: 86; and
(c) CDR-H3 comprising the amino acid sequence of SEQ ID NO: 87;
or
(4)
(a) CDR-H1 comprising the amino acid sequence of SEQ ID NO: 85;
(b) CDR-H2 comprising the amino acid sequence of SEQ ID NO: 89; and
(c) CDR-H3 comprising the amino acid sequence of SEQ ID NO: 87.

In one preferred aspect, the antibody comprises CDR-H3 comprising the amino acid sequence of SEQ ID NO: 87.

In another aspect, the invention provides an antibody comprising at least one, at least two, or all three VL CDR sequences selected from
(1)
(a) CDR-L1 comprising the amino acid sequence of SEQ ID NO: 81;
(b) CDR-L2 comprising the amino acid sequence of SEQ ID NO: 82; and
(c) CDR-L3 comprising the amino acid sequence of SEQ ID NO: 83;
or
(2)
(a) CDR-L1 comprising the amino acid sequence of SEQ ID NO: 81;
(b) CDR-L2 comprising the amino acid sequence of SEQ ID NO: 82; and
(c) CDR-L3 comprising the amino acid sequence of SEQ ID NO: 83;
or
(3)
(a) CDR-L1 comprising the amino acid sequence of SEQ ID NO: 81;
(b) CDR-L2 comprising the amino acid sequence of SEQ ID NO: 82; and
(c) CDR-L3 comprising the amino acid sequence of SEQ ID NO: 91;
or
(4)
(a) CDR-L1 comprising the amino acid sequence of SEQ ID NO: 81;
(b) CDR-L2 comprising the amino acid sequence of SEQ ID NO: 82; and
(c) CDR-L3 comprising the amino acid sequence of SEQ ID NO: 91.

In another aspect, an antibody of the invention comprises
(a) a VH domain comprising at least one, at least two, or all three VH CDR sequences selected from
   (1)
   (a) CDR-H1 comprising the amino acid sequence of SEQ ID NO: 85;
   (b) CDR-H2 comprising the amino acid sequence of SEQ ID NO: 86; and
   (c) CDR-H3 comprising the amino acid sequence of SEQ ID NO: 87;
   or
   (2)
   (a) CDR-H1 comprising the amino acid sequence of SEQ ID NO: 85;
   (b) CDR-H2 comprising the amino acid sequence of SEQ ID NO: 89; and
   (c) CDR-H3 comprising the amino acid sequence of SEQ ID NO: 87;
   or
   (3)
   (a) CDR-H1 comprising the amino acid sequence of SEQ ID NO: 85;
   (b) CDR-H2 comprising the amino acid sequence of SEQ ID NO: 86; and
   (c) CDR-H3 comprising the amino acid sequence of SEQ ID NO: 87;
   or
   (4)
   (a) CDR-H1 comprising the amino acid sequence of SEQ ID NO: 85;
   (b) CDR-H2 comprising the amino acid sequence of SEQ ID NO: 89; and
   (c) CDR-H3 comprising the amino acid sequence of SEQ ID NO: 87,
and (b) a VL domain comprising at least one, at least two, or all three VL CDR sequences selected from
   (1)
   (a) CDR-L1 comprising the amino acid sequence of SEQ ID NO: 81;
   (b) CDR-L2 comprising the amino acid sequence of SEQ ID NO: 82; and
   (c) CDR-L3 comprising the amino acid sequence of SEQ ID NO: 83;
   or
   (2)
   (a) CDR-L1 comprising the amino acid sequence of SEQ ID NO: 81;
   (b) CDR-L2 comprising the amino acid sequence of SEQ ID NO: 82; and
   (c) CDR-L3 comprising the amino acid sequence of SEQ ID NO: 83;
   or
   (3)
   (a) CDR-L1 comprising the amino acid sequence of SEQ ID NO: 81;
   (b) CDR-L2 comprising the amino acid sequence of SEQ ID NO: 82; and
   (c) CDR-L3 comprising the amino acid sequence of SEQ ID NO: 91;
   or
   (4)
   (a) CDR-L1 comprising the amino acid sequence of SEQ ID NO: 81;
   (b) CDR-L2 comprising the amino acid sequence of SEQ ID NO: 82; and
   (c) CDR-L3 comprising the amino acid sequence of SEQ ID NO: 91.

In another aspect, the invention provides an anti-A-beta protein antibody comprising
(1)
(a) CDR-H1 comprising the amino acid sequence of SEQ ID NO: 85;
(b) CDR-H2 comprising the amino acid sequence of SEQ ID NO: 86;
(c) CDR-H3 comprising the amino acid sequence of SEQ ID NO: 87;
(d) CDR-L1 comprising the amino acid sequence of SEQ ID NO: 81;
(e) CDR-L2 comprising the amino acid sequence of SEQ ID NO: 82; and
(f) CDR-L3 comprising the amino acid sequence of SEQ ID NO: 83;
or
(2)
(a) CDR-H1 comprising the amino acid sequence of SEQ ID NO: 85;
(b) CDR-H2 comprising the amino acid sequence of SEQ ID NO: 89;
(c) CDR-H3 comprising the amino acid sequence of SEQ ID NO: 87;
(d) CDR-L1 comprising the amino acid sequence of SEQ ID NO: 81;
(e) CDR-L2 comprising the amino acid sequence of SEQ ID NO: 82; and (f) CDR-L3 comprising the amino acid sequence of SEQ ID NO: 83;
or
(3)
(a) CDR-H1 comprising the amino acid sequence of SEQ ID NO: 85;
(b) CDR-H2 comprising the amino acid sequence of SEQ ID NO: 86;
(c) CDR-H3 comprising the amino acid sequence of SEQ ID NO: 87;
(d) CDR-L1 comprising the amino acid sequence of SEQ ID NO: 81;
(e) CDR-L2 comprising the amino acid sequence of SEQ ID NO: 82; and
(f) CDR-L3 comprising the amino acid sequence of SEQ ID NO: 91;
or
(4)
(a) CDR-H1 comprising the amino acid sequence of SEQ ID NO: 85;
(b) CDR-H2 comprising the amino acid sequence of SEQ ID NO: 89;
(c) CDR-H3 comprising the amino acid sequence of SEQ ID NO: 87;
(d) CDR-L1 comprising the amino acid sequence of SEQ ID NO: 81;
(e) CDR-L2 comprising the amino acid sequence of SEQ ID NO: 82; and
(f) CDR-L3 comprising the amino acid sequence of SEQ ID NO: 91.

In another aspect, an anti-A-beta protein antibody according to the invention comprises one or more of the CDR sequences of the VH of SEQ ID NO: 84 or SEQ ID NO: 88. In another embodiment, an anti-A-beta protein antibody comprises one or more of the CDR sequences of the VL of SEQ ID NO: 80 or SEQ ID NO: 90. In another embodiment, an anti-A-beta antibody comprises (1) the CDR sequences of the VH of SEQ ID NO: 84 and the CDR sequences of the VL of SEQ ID NO: 80; or (2) the CDR sequences of the VH of SEQ ID NO: 88 and the CDR sequences of the VL of SEQ ID NO: 80; or (3) the CDR sequences of the VH of SEQ ID NO: 84 and the CDR sequences of the VL of SEQ ID NO: 90; or (4) the CDR sequences of the VH of SEQ ID NO: 88 and the CDR sequences of the VL of SEQ ID NO: 90.

In one preferred aspect, an anti-A-beta protein antibody comprises the CDR-H1, CDR-H2 and CDR-H3 amino acid sequences of the VH domain of SEQ ID NO: 84 and the CDR-L1, CDR-L2 and CDR-L3 amino acid sequences of the VL domain of SEQ ID NO: 80.

In one aspect, an anti-A-beta protein antibody according to the current invention comprises one or more of the heavy chain CDR amino acid sequences of the VH domain of SEQ ID NO: 84 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO 84. In one aspect, the anti-A-beta protein antibody according to the current invention comprises the three heavy chain CDR amino acid sequences of the VH domain of SEQ ID NO: 84 and a framework of at least 85%, 86%, 87%, 88%, 89%, 90%, 91%, 92%, 93%, 94%, 95%, 96%, 97%, 98%, 99% sequence identity to the framework amino acid sequence of the VH domain of SEQ ID NO: 84. In one aspect, the anti-A-beta protein antibody comprises the three heavy chain CDR amino acid sequences of the VH domain of SEQ ID NO: 84 and a framework of at least 95% sequence identity to the framework amino acid sequence of the VH domain of SEQ ID NO: 84. In another aspect, the anti-A-beta protein antibody comprises the three heavy chain CDR amino acid sequences of the VH domain of SEQ ID NO: 84 and a framework of at least of at least 98% sequence identity to the framework amino acid sequence of the VH domain of SEQ ID NO: 84.

In one aspect, an anti-A-beta protein antibody comprises one or more of the light chain CDR amino acid sequences of the VL domain of SEQ ID NO: 80 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 80. In one aspect, the anti-A-beta protein antibody comprises the three light chain CDR amino acid sequences of the VL domain of SEQ ID NO: 80 and a framework of at least 85%, 86%, 87%, 88%, 89%, 90%, 91%, 92%, 93%, 94%, 95%, 96%, 97%, 98%, 99% sequence identity to the framework amino acid sequence of the VL domain of SEQ ID NO: 80. In one aspect, the anti-A-beta protein antibody comprises the three light chain CDR amino acid sequences of the VL domain of SEQ ID NO: 80 and a framework of at least 95% sequence identity to the framework amino acid sequence of the VL domain of SEQ ID NO: 80. In another aspect, the anti-A-beta protein antibody comprises the three light chain CDR amino acid sequences of the VL domain of SEQ ID NO: 80 and a framework of at least particularly of at least 98% sequence identity to the framework amino acid sequence of the VH domain of SEQ ID NO: 80.

In one aspect, the anti-A-beta protein antibody comprises (a) CDR-H1 comprising the amino acid sequence of SEQ ID NO: 85; (b) CDR-H2 comprising the amino acid sequence of SEQ ID NO: 86; (c) CDR-H3 comprising the amino acid sequence of SEQ ID NO: 87; (d) CDR-L1 comprising the amino acid sequence of SEQ ID NO: 81; (e) CDR-L2 comprising the amino acid sequence of SEQ ID NO: 82; and (f) CDR-L3 comprising the amino acid sequence of SEQ ID NO: 83, and a VH domain having at least 90%, 91%, 92%, 93%, 94%, 95%, 96%, 97%, 98%, 99%, or 100% sequence identity to the amino acid sequence of SEQ ID NO: 84, and a VL domain having at least 90%, 91%, 92%, 93%, 94%, 95%, 96%, 97%, 98%, 99%, or 100% sequence identity to the amino acid sequence of SEQ ID NO: 80. In one preferred embodiment, the VH domain has at least 95% sequence identity to the amino acid sequence of SEQ ID NO: 84 and the VL domain has at least 95% sequence identity to the amino acid sequence of SEQ ID NO: 80.

In one aspect, the anti-A-beta protein antibody comprises (a) CDR-H1 comprising the amino acid sequence of SEQ ID NO: 85; (b) CDR-H2 comprising the amino acid sequence of SEQ ID NO: 86; (c) CDR-H3 comprising the amino acid sequence of SEQ ID NO: 87; (d) CDR-L1 comprising the amino acid sequence of SEQ ID NO: 81; (e) CDR-L2 comprising the amino acid sequence of SEQ ID NO: 82; and (f) CDR-L3 comprising the amino acid sequence of SEQ ID NO: 83, and a VH domain having at least 90%, 91%, 92%, 93%, 94%, 95%, 96%, 97%, 98%, 99%, or 100% sequence identity to the amino acid sequence of SEQ ID NO: 84, and a VL domain having at least 90%, 91%, 92%, 93%, 94%, 95%, 96%, 97%, 98%, 99%, or 100% sequence identity to the amino acid sequence of SEQ ID NO: 80; wherein the antibody specifically binds to human A-beta protein. In one preferred embodiment, the VH domain has at least 95% sequence identity to the amino acid sequence of SEQ ID NO: 84 and the VL domain has at least 95% sequence identity to the amino acid sequence of SEQ ID NO: 80.

In another aspect, an anti-A-beta protein antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 84. In one aspect, an anti-A-beta protein antibody comprises a heavy chain variable domain (VH) sequence having at least 95%, sequence identity to the amino acid sequence of SEQ ID NO: 84. In certain aspects, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-A-beta protein antibody comprising that sequence retains the ability to bind to human A-beta protein. In certain aspects, a total of 1 to 10 amino acids have been substituted, inserted and/or deleted in SEQ ID NO: 84. In certain aspects, substitutions, insertions, or deletions occur in regions outside the CDRs (i.e., in the FRs). Optionally, the anti-A-beta protein antibody comprises the VH sequence in SEQ ID NO: 84, including post-translational modifications of that sequence. In a particular aspect, the VH comprises one, two or three CDRs selected from: (a) CDR-H1, comprising the amino acid sequence of SEQ ID NO: 85, (b) CDR-H2, comprising the amino acid sequence of SEQ ID NO: 86, and (c) CDR-H3, comprising the amino acid sequence of SEQ ID NO: 87. In another aspect, an anti-A-beta protein antibody is provided, wherein the antibody comprises a light chain variable domain (VL) sequence having at least 90%, 91%, 92%, 93%, 94%, 95%, 96%, 97%, 98%, 99%, or 100% sequence identity to the amino acid sequence of SEQ ID NO: 80. In one aspect, an anti-A-beta protein antibody comprises a light chain variable domain (VL) sequence having at least 95% sequence identity to the amino acid sequence of SEQ ID NO: 80. In certain aspects, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-A-beta protein antibody comprising that sequence retains the ability to bind to human A-beta protein. In certain aspects, a total of 1 to 10 amino acids have been substituted, inserted and/or deleted in SEQ ID NO: 80. In certain aspects, the substitutions, insertions, or deletions occur in regions outside the CDRs (i.e., in the FRs). Optionally, the anti-A-beta protein antibody comprises the VL sequence in SEQ ID NO: 80, including post-translational modifications of that sequence. In a particular aspect, the VL comprises one, two or three CDRs selected from: (a) CDR-L1, comprising the amino acid sequence of SEQ ID NO: 81, (b) CDR-L2, comprising the amino acid sequence of SEQ ID NO: 82, and (c) CDR-L3, comprising the amino acid sequence of SEQ ID NO: 83.

In another aspect, an anti-A-beta protein antibody is provided, wherein the antibody comprises a VH sequence as in any of the aspects provided above, and a VL sequence as in any of the aspects provided above. In one aspect, the antibody comprises the VH and VL sequences in SEQ ID NO: 84 and SEQ ID NO: 80, respectively, including post-translational modifications of those sequences.

In a further aspect of the invention, an anti-A-beta protein antibody according to any of the above aspects is a monoclonal antibody, including a chimeric, humanized or human antibody. In one aspect, an anti-A-beta protein antibody is an antibody fragment, e.g., an Fv, Fab, Fab′, scFv, diabody, or F(ab′)2 fragment. In another aspect, the antibody is a full-length antibody, e.g., an intact IgG1 antibody or other antibody class or isotype as defined herein.

| antibody | sequence | SEQ ID NO: |
|---|---|---|
| mAb 675 VL (kappa) | DIVLTQSPATLSLSPGERATLSC | 80 |
| | RASQSVSSSYLAWYQQKPGQA | |
| | PRLLIYGASSRATGVPARFSGSG | |
| | SGTDFTLTISSLEPEDFATYYCL | |
| | QIYNMPITFGQGTKVEIK | |
| mAb 675 VL (CDR1) | RASQSVSSSYLA | 81 |
| mAb 675 VL (CDR2) | GASSRAT | 82 |
| mAb 675 VL (CDR3) | LQIYNMPIT | 83 |
| mAb 675 VH | QVELVESGGGLVQPGGSLRLSC | 84 |
| | AASGFTFSSYAMSWVRQAPGK | |
| | GLEWVSAINATGTRTYYADSV | |
| | KGRFTISRDNSKNTLYLQMNSL | |
| | RAEDTAVYYCARGKGSSGYVR | |
| | YFDVWGQGTLVTVSS | |
| mAb 675 VH (CDR1) | GFTFSSYAMS | 85 |
| mAb 675 VH (CDR2) | AINATGTRTYYADSVKG | 86 |
| mAb 675 VH (CDR3) | GKGSSGYVRYFDV | 87 |
| mAb 663 VL (kappa) | DIVLTQSPATLSLSPGERATLSC | 80 |
| | RASQSVSSSYLAWYQQKPGQA | |
| | PRLLIYGASSRATGVPARFSGSG | |
| | SGTDFTLTISSLEPEDFATYYCL | |
| | QIYNMPITFGQGTKVEIK | |
| mAb 663 VL (CDR1) | RASQSVSSSYLA | 81 |
| mAb 663 VL (CDR2) | GASSRAT | 82 |
| mAb 663 VL (CDR3) | LQIYNMPIT | 83 |
| mAb 663 VH | QVELVESGGGLVQPGGSLRLSC | 88 |
| | AASGFTFSSYAMSWVRQAPGK | |
| | GLEWVSAIQASGTRTYYADSVK | |
| | GRFTISRDNSKNTLYLQMNSLR | |
| | AEDTAVYYCARGKGSSGYVRY | |
| | FDVWGQGTLVTVSS | |
| mAb 663 VH (CDR1) | GFTFSSYAMS | 85 |
| mAb 663 VH (CDR2) | AIQASGTRTYYADSVKG | 89 |
| mAb 663 VH (CDR3) | GKGSSGYVRYFDV | 87 |
| mAb 651 VL (kappa) | DIVLTQSPATLSLSPGERATLSC | 90 |
| | RASQSVSSSYLAWYQQKPGQA | |
| | PRLLIYGASSRATGVPARFSGSG | |
| | SGTDFTLTISSLEPEDFATYYCL | |
| | QIYNHPITFGQGTKVEIK | |
| mAb 651 VL (CDR1) | RASQSVSSSYLA | 81 |
| mAb 651 VL (CDR2) | GASSRAT | 82 |
| mAb 651 VL (CDR3) | LQIYNHPIT | 91 |
| mAb 651 VH | QVELVESGGGLVQPGGSLRLSC | 84 |
| | AASGFTFSSYAMSWVRQAPGK | |
| | GLEWVSAINATGTRTYYADSV | |
| | KGRFTISRDNSKNTLYLQMNSL | |
| | RAEDTAVYYCARGKGSSGYVR | |
| | YFDVWGQGTLVTVSS | |
| mAb 651 VH (CDR1) | GFTFSSYAMS | 85 |
| mAb 651 VH (CDR2) | AINATGTRTYYADSVKG | 86 |
| mAb 651 VH (CDR3) | GKGSSGYVRYFDV | 87 |
| mAb 638 VL (kappa) | DIVLTQSPATLSLSPGERATLSC | 90 |
| | RASQSVSSSYLAWYQQKPGQA | |
| | PRLLIYGASSRATGVPARFSGSG | |
| | SGTDFTLTISSLEPEDFATYYCL | |
| | QIYNHPITFGQGTKVEIK | |
| mAb 638 VL (CDR1) | RASQSVSSSYLA | 81 |
| mAb 638 VL (CDR2) | GASSRAT | 82 |
| mAb 638 VL (CDR3) | LQIYNHPIT | 91 |
| mAb 638 VH | QVELVESGGGLVQPGGSLRLSC | 88 |
| | AASGFTFSSYAMSWVRQAPGK | |
| | GLEWVSAIQASGTRTYYADSVK | |
| | GRFTISRDNSKNTLYLQMNSLR | |
| | AEDTAVYYCARGKGSSGYVRY | |
| | FDVWGQGTLVTVSS | |
| mAb 638 VH (CDR1) | GFTFSSYAMS | 85 |
| mAb 638 VH (CDR2) | AIQASGTRTYYADSVKG | 89 |
| mAb 638 VH (CDR3) | GKGSSGYVRYFDV | 87 |
| mAb 675, mAb 663, | DIVLTQSPAT LSLSPGERAT | 80 |
| gantenerumab VL | LSCRASQSVS SSYLAWYQQK | |
| | PGQAPRLLIY GASSRATGVP | |
| | ARFSGSGSGT DFTLTISSLE | |
| | PEDFATYYCL QIYNMPITFG | |
| | QGTKVEIK | |
| mAb 675, mAb 663, | RASQSVSSSYLA | 81 |
| gantenerumab VL | | |
| (CDR1) | | |
| mAb 675, mAb 663, | GASSRAT | 82 |
| gantenerumab VL | | |
| (CDR2) | | |
| gantenerumab VL | LQIYNMPIT | 83 |
| (CDR3) | | |
| gantenerumab VH | QVELVESGGG LVQPGGSLRL | 76 |
| | SCAASGFTFS SYAMSWVRQA | |
| | PGKGLEWVSA INASGTRTYY | |
| | ADSVKGRFTI SRDNSKNTLY | |
| | LQMNSLRAED TAVYYCARGK | |
| | GNTHKPYGYV RYFDVWGQGT | |
| | LVTVSS | |
| mAb 675, mAb 663, | GFTFSSYAMS | 77, 85 |
| mAb 651, mAb 638, | | |
| gantenerumab VH | | |
| (CDR1) | | |
| gantenerumab VH | AINASGTRTYYADSVKG | 78 |
| (CDR2) | | |
| gantenerumab VH | GKGNTHKPYGYVRYFDV | 79 |
| (CDR3) | | |

In certain embodiments, amino acid sequence variants of the antibody according to the current invention are contemplated. For example, it may be desirable to improve the binding affinity and/or other biological properties of the antibody according to the current invention. Amino acid sequence variants of an antibody according to the current invention may be prepared by introducing appropriate modifications into the nucleotide sequence encoding the antibody according to the current invention, or by peptide synthesis. Such modifications include, for example, deletions from, and/or insertions into and/or substitutions of residues within the amino acid sequences of the antibody according to the current invention. Any combination of deletion, insertion, and substitution can be made to arrive at the final construct, provided that the final construct possesses the desired characteristics, e.g., antigen-binding.

In certain embodiments of all aspects and embodiments according to the current invention, the heavy chain variable domain of the antibody according to the current invention comprises as first residue instead of a glutamine (Q) residue a pyroglutamic acid (pE) residue.

### a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in the Table below under the heading of “preferred substitutions”. More substantial changes are provided in the Table below under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity-matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, P. S., Methods Mol. Biol. 207 (2008) 179-196), and/or residues that contact antigen, with the resulting variant VH or VL being tested for binding affinity. Affinity maturation by constructing and reselecting from secondary libraries has been described, e.g., in Hoogenboom, H. R. et al. in Methods in Molecular Biology 178 (2002) 1-37. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody according to the current invention to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham, B. C. and Wells, J. A., Science 244 (1989) 1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

### b) Fc-Region Glycosylation Variants

In certain embodiments, an antibody according to the current invention is altered to increase or decrease the extent to which the antibody Fc-region is glycosylated. Addition or deletion of glycosylation sites to an antibody according to the current invention may be conveniently accomplished by altering the amino acid sequence such that one or more glycosylation sites is created or removed.

Where the antibody comprises an Fc-region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright, A. and Morrison, S. L., TIBTECH 15 (1997) 26-32. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody as reported herein may be made in order to create antibody variants with certain improved properties.

In certain embodiments, antibody variants are provided having a carbohydrate structure that lacks fucose attached (directly or indirectly) to the Fc-region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US 2003/0157108; US 2004/0093621. Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO 2005/053742; WO 2002/031140; Okazaki, A. et al., J. Mol. Biol. 336 (2004) 1239-1249; Yamane-Ohnuki, N. et al., Biotech.

Bioeng. 87 (2004) 614-622. Examples of cell lines capable of producing defucosylated antibodies include Lec13 CHO cells deficient in protein fucosylation (Ripka, J. et al., Arch. Biochem. Biophys. 249 (1986) 533-545; US 2003/0157108; and WO 2004/056312, especially at Example 11), and knockout cell lines, such as alpha-1,6-fucosyltransferase gene, FUT8, knockout CHO cells (see, e.g., Yamane-Ohnuki, N. et al., Biotech. Bioeng. 87 (2004) 614-622; Kanda, Y. et al., Biotechnol. Bioeng. 94 (2006) 680-688; and WO 2003/085107).

Antibodies variants are further provided with bisected oligosaccharides, e.g., in which a biantennary oligosaccharide attached to the Fc-region of the antibody is bisected by GlcNAc. Such antibody variants may have reduced fucosylation and/or improved ADCC function. Examples of such antibody variants are described, e.g., in WO 2003/011878; U.S. Pat. No. 6,602,684; and US 2005/0123546. Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

### c) Fc-Region Variants

In certain embodiments, one or more amino acid modifications may be introduced into the Fc-region of an antibody according to the current invention, thereby generating an Fc-region variant. The Fc-region variant may comprise a human Fc region sequence (e.g., a human IgG1, IgG2, IgG3 or IgG4 Fc region) comprising an amino acid modification (e.g. a substitution) at one or more amino acid positions.

In certain embodiments, herein is contemplated an antibody variant that possesses some but not all effector functions, which make it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch, J. V. and Kinet, J. P., Annu. Rev. Immunol. 9 (1991) 457-492. Non-limiting examples of in vitro assays to assess ADCC activity of a molecule of interest is described in U.S. Pat. No. 5,500,362 (see, e.g. Hellstrom, I. et al., Proc. Natl. Acad. Sci. USA 83 (1986) 7059-7063; and Hellstrom, I. et al., Proc. Natl. Acad. Sci. USA 82 (1985) 1499-1502); U.S. Pat. No. 5,821,337 (see Bruggemann, M. et al., J. Exp. Med. 166 (1987) 1351-1361). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes, R. et al., Proc. Natl. Acad. Sci. USA 95 (1998) 652-656. C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro, H. et al., J. Immunol. Methods 202 (1996) 163-171; Cragg, M. S. et al., Blood 101 (2003) 1045-1052; and Cragg, M. S. and M. J. Glennie, Blood 103 (2004) 2738-2743). FcRn binding and in vivo clearance/half-life determinations can also be performed using methods known in the art (see, e.g., Petkova, S. B. et al., Int. Immunol. 18 (2006: 1759-1769).

Antibodies with reduced effector function include those with substitution of one or more of Fc-region residues 238, 265, 269, 270, 297, 327 and 329 (U.S. Pat. No. 6,737,056). Such Fc-region variants include Fc-region variants with substitutions at two or more of amino acid positions 265, 269, 270, 297 and 327, including the so-called “DANA” Fc-region variant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields, R. L. et al., J. Biol. Chem. 276 (2001) 6591-6604).

In certain embodiments, an antibody variant comprises an Fc-region with one or more amino acid substitutions that improve ADCC, e.g., substitutions at positions 298, 333, and/or 334 of the Fc region (EU numbering of residues).

In some embodiments, alterations are made in the Fc-region that result in altered (i.e., either improved or diminished) C1q binding and/or Complement Dependent Cytotoxicity (CDC), e.g., as described in U.S. Pat. No. 6,194,551, WO 99/51642, and Idusogie, E. E. et al., J. Immunol. 164 (2000) 4178-4184.

Antibodies with increased half-lives and improved binding to the neonatal Fc receptor (FcRn), which is responsible for the transfer of maternal IgGs to the fetus (Guyer, R. L. et al., J. Immunol. 117 (1976) 587-593, and Kim, J. K. et al., J. Immunol. 24 (1994) 2429-2434), are described in US 2005/0014934. Those antibodies comprise an Fc-region with one or more substitutions therein which improve binding of the Fc-region to FcRn. Such Fc-region variants include those with substitutions at one or more of Fc-region residues: 238, 256, 265, 272, 286, 303, 305, 307, 311, 312, 317, 340, 356, 360, 362, 376, 378, 380, 382, 413, 424 or 434, e.g., substitution of Fc-region residue 434 (U.S. Pat. No. 7,371,826).

See also Duncan, A. R. and Winter, G., Nature 322 (1988) 738-740; U.S. Pat. Nos. 5,648,260; 5,624,821; and WO 94/29351 concerning other examples of Fc-region variants.

### d) Cysteine Engineered Antibody Variants

In certain embodiments, it may be desirable to create cysteine-engineered antibodies, e.g., “thioMAbs,” in which one or more residues of an antibody are substituted with cysteine residues. In certain embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

### e) Antibody Derivatives

In certain embodiments, an antibody according to the current invention may be further modified to contain additional non-proteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, polypropylene oxide/ethylene oxide copolymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In certain embodiments, conjugates of an antibody and a non-proteinaceous moiety that may be selectively heated by exposure to radiation are provided. In certain embodiments, the non-proteinaceous moiety is a carbon nanotube (Kam, N. W. et al., Proc. Natl. Acad. Sci. USA 102 (2005) 11600-11605). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed.

### Recombinant Methods and Compositions

Antibodies according to the current invention may be produced using recombinant methods and compositions, e.g., as described in U.S. Pat. No. 4,816,567. The current invention provides one or more isolated nucleic acid molecules encoding an anti-human A-beta antibody according to the current invention. Such nucleic acid molecules may encode an amino acid sequence comprising the VL and/or an amino acid sequence comprising the VH of the antibody according to the current invention (e.g., the light and/or heavy chains of the antibody according to the current invention). In certain embodiments, one or more plasmids (e.g., expression plasmids) comprising such nucleic acid molecules are provided. In certain embodiments, a host cell comprising such nucleic acid molecules is provided. In one preferred embodiment, a host cell comprises (e.g., has been transformed with): (1) a plasmid comprising a first nucleic acid molecule that encodes an amino acid sequence comprising the VL of the antibody and a second nucleic acid molecule that encodes an amino acid sequence comprising the VH of the antibody, or (2) a first plasmid comprising a nucleic acid molecule that encodes an amino acid sequence comprising the VL of the antibody and a second plasmid comprising a nucleic acid molecule that encodes an amino acid sequence comprising the VH of the antibody. In certain embodiments, the host cell is eukaryotic, e.g. a Chinese Hamster Ovary (CHO) cell or lymphoid cell (e.g., Y0, NS0, Sp2/0 cell). In certain embodiments, a method of making an anti-human A-beta protein antibody is provided, wherein the method comprises culturing a host cell comprising one or more nucleic acid molecules encoding the antibody, as provided above, under conditions suitable for expression of the antibody, and optionally recovering the antibody from the host cell (or host cell culture medium).

For recombinant production of an anti-human A-beta protein antibody, nucleic acid molecules encoding an antibody, e.g., as described above, are isolated and inserted into one or more plasmids for further cloning and/or expression in a host cell. Such nucleic acid molecules may be readily isolated and sequenced using conventional procedures (e.g., by using oligonucleotide probes that are capable of binding specifically to genes encoding the heavy and light chains of the antibody).

Suitable host cells for cloning or expression of antibody-encoding nucleic acid molecules include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of glycosylated antibodies are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather, J. P. et al., Annals N.Y. Acad. Sci. 383 (1982) 44-68; MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NSO and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

### Methods and Compositions for Diagnostics and Detection

In certain embodiments, any of the anti-human A-beta protein antibodies according to the current invention are useful for detecting the presence of the A-beta protein in a biological sample. The term “detecting” as used herein encompasses quantitative or qualitative detection. In certain embodiments, a biological sample comprises a cell or tissue.

In certain embodiments, an anti-human A-beta protein antibody according to the current invention for use in a method of diagnosis or detection is provided. In certain embodiments, a method of detecting the presence of the A-beta protein in a biological sample is provided. In certain embodiments, the method comprises contacting the biological sample with an anti-human A-beta protein antibody according to the current invention under conditions permissive for binding of the anti-human A-beta protein antibody to the A-beta protein, and detecting whether a complex is formed between the anti-human A-beta protein antibody and the A-beta protein. Such method may be an in vitro or in vivo method.

In certain embodiments, labeled anti-human A-beta protein antibodies according to the current invention are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes 32P, 14C, 1251, 3H, and 1311, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, 0-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

### Pharmaceutical Formulations

Pharmaceutical formulations of an anti-human A-beta antibody according to the current invention are prepared by mixing such antibody having the desired degree of purity with one or more optional pharmaceutically acceptable carriers (Remington's Pharmaceutical Sciences, 16th edition, Oslo, A. (ed.) (1980)), in the form of lyophilized formulations or aqueous solutions. Pharmaceutically acceptable carriers are generally non-toxic to recipients at the dosages and concentrations employed, and include, but are not limited to: buffers such as phosphate, citrate, and other organic acids; antioxidants including ascorbic acid and methionine; preservatives (such as octadecyl dimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as poly(vinylpyrrolidone); amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rhuPH20 (HYLENEX®, Baxter International, Inc.). Certain exemplary sHASEGPs and methods of use, including rhuPH20, are described in US 2005/0260186 and US 2006/0104968. In certain embodiments, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO 2006/044908, the latter formulations including a histidine-acetate buffer.

The formulation herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methyl methacrylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences, 16th edition, Oslo, A. (ed.) (1980).

Sustained-release preparations may be prepared. Suitable examples of sustained-release preparations include semi-permeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration are generally sterile. Sterility may be readily accomplished, e.g., by filtration through sterile filtration membranes. In one embodiment, the formulation is isotonic.

### Therapeutic Methods and Compositions

Any of the anti-human A-beta protein antibodies according to the current invention may be used in therapeutic methods.

In one aspect, an anti-human A-beta protein antibody according to the current invention for use as a medicament is provided. In further aspects, an anti-human A-beta antibody according to the current invention for use in preventing and/or treating a disease associated with amyloidogenesis and/or amyloid-plaque formation is provided. In certain embodiments, an anti-human A-beta protein antibody according to the current invention for use in a method of treatment is provided. In certain embodiments, herein is provided an anti-human A-beta protein antibody according to the current invention for use in a method of treating an individual having a disease associated with amyloidogenesis and/or amyloid-plaque formation comprising administering to the individual an effective amount of the anti-human A-beta protein antibody according to the current invention. In certain embodiments, the method further comprises administering to the individual an effective amount of at least one additional therapeutic agent, such as listed below or an anti-pTau or an anti-alpha-synuclein antibody. In further embodiments, herein is provided an anti-human A-beta protein antibody according to the current invention for use in inhibiting the formation of plaques and/or disintegrating β-amyloid plaques. In certain embodiments, herein is provided an anti-human A-beta protein antibody according to the current invention for use in a method of inhibiting the formation of plaques and/or disintegrating β-amyloid plaques in an individual comprising administering to the individual an effective of the anti-human A-beta protein antibody according to the current invention to inhibit the formation of plaques and/or to disintegrate β-amyloid plaques. An “individual” according to any of the above embodiments is preferably a human.

In a further aspect, herein is provided the use of an anti-human A-beta antibody according to the current invention in the manufacture or preparation of a medicament. In certain embodiments, the medicament is for treatment of a disease associated with amyloidogenesis and/or amyloid-plaque formation. In certain embodiments, the medicament is for use in a method of treating a disease associated with amyloidogenesis and/or amyloid-plaque formation comprising administering to an individual having a disease associated with amyloidogenesis and/or amyloid-plaque formation an effective amount of the medicament. In certain embodiments, the method further comprises administering to the individual an effective amount of at least one additional therapeutic agent, such as listed below or an anti-pTau or an anti-alpha synuclein antibody. In certain embodiments, the medicament is for the inhibition of the formation of plaques and/or the disintegration of β-amyloid plaques.

In certain embodiments, the medicament is for use in a method of inhibiting the formation of plaques and/or the disintegration of β-amyloid plaques in an individual comprising administering to the individual an amount effective of the medicament to inhibit the formation of plaques and/or to disintegrate β-amyloid plaques. An “individual” according to any of the above embodiments may be a human.

In a further aspect, herein is provided a method for treating a disease associated with amyloidogenesis and/or amyloid-plaque formation. In certain embodiments, the method comprises administering to an individual having a disease associated with amyloidogenesis and/or amyloid-plaque formation an effective amount of an anti-human A-beta protein antibody according to the current invention. In certain embodiments, the method further comprises administering to the individual an effective amount of at least one additional therapeutic agent, such as given below or an anti-pTau or an anti-alpha-synuclein antibody. An “individual” according to any of the above embodiments may be a human.

In a further aspect, herein is provided a method for inhibiting the formation of plaques and/or for disintegrating β-amyloid plaques in an individual. In certain embodiments, the method comprises administering to the individual an effective amount of an anti-human A-beta protein antibody according to the current invention to inhibit the formation of plaques and/or to disintegrate β-amyloid plaques. In one embodiment, an “individual” is a human.

In a further aspect, herein are provided pharmaceutical formulations comprising any of the anti-human A-beta protein antibodies according to the current invention, e.g., for use in any of the above therapeutic methods. In certain embodiments, a pharmaceutical formulation comprises any of the anti-human A-beta protein antibodies according to the current invention and a pharmaceutically acceptable carrier. In certain embodiments, a pharmaceutical formulation comprises any of the anti-human A-beta protein antibodies according to the current invention and at least one additional therapeutic agent, e.g., as given below or an anti-pTau or an anti-alpha-synuclein antibody.

Antibodies according to the current invention can be used either alone or in combination with other agents in a therapy. For instance, an antibody according to the current invention may be co-administered with at least one additional therapeutic agent. In certain embodiments, an additional therapeutic agent is a therapeutic agent effective to treat the same or a different neurological disorder as the antibody according to the current invention is being employed to treat. Exemplary additional therapeutic agents include, but are not limited to: the various neurological drugs described above, cholinesterase inhibitors (such as donepezil, galantamine, rovastigmine, and tacrine), NMDA receptor antagonists (such as memantine), amyloid beta peptide aggregation inhibitors, antioxidants, γ-secretase modulators, nerve growth factor (NGF) mimics or NGF gene therapy, PPARy agonists, HMS-CoA reductase inhibitors (statins), ampakines, calcium channel blockers, GABA receptor antagonists, glycogen synthase kinase inhibitors, intravenous immunoglobulin, muscarinic receptor agonists, nicrotinic receptor modulators, active or passive amyloid beta peptide immunization, phosphodiesterase inhibitors, serotonin receptor antagonists and anti-amyloid beta peptide antibodies. In certain embodiments, the at least one additional therapeutic agent is selected for its ability to mitigate one or more side effects of the neurological drug.

Such combination therapies noted above encompass combined administration (where two or more therapeutic agents are included in the same or separate formulations), and separate administration, in which case, administration of the antibody as reported herein can occur prior to, simultaneously, and/or following, administration of the additional therapeutic agent or agents. In certain embodiments, administration of the anti-human A-beta antibody according to the current invention and administration of an additional therapeutic agent occur within about one month, or within about one, two or three weeks, or within about one, two, three, four, five, or six days, of each other. Antibodies according to the current invention can also be used in combination with other interventional therapies such as, but not limited to, radiation therapy, behavioral therapy, or other therapies known in the art and appropriate for the neurological disorder to be treated or prevented.

An antibody according to the current invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies according to the current invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody according to the current invention present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

Lipid-based methods of transporting the antibody according to the current invention or a fusion construct comprising the antibody according to the current invention across the BBB include, but are not limited to, encapsulating the antibody or the fusion construct in liposomes that are coupled to monovalent binding entity that bind to receptors on the vascular endothelium of the BBB (see e.g., US 2002/0025313), and coating the monovalent binding entity in low-density lipoprotein particles (see e.g., US 2004/0204354) or apolipoprotein E (see e.g., US 2004/0131692).

For the prevention or treatment of disease, the appropriate dosage of an antibody according to the current invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody according to the current invention is suitably administered to the patient at one time or over a series of treatments. Depending on the type and severity of the disease, about 1 μg/kg to 15 mg/kg (e.g. 0.5 mg/kg-10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. However, other dosage regimens may be useful. The progress of this therapy is easily monitored by conventional techniques and assays.

It is understood that any of the above formulations or therapeutic methods may be carried out using an immunoconjugate as reported herein in place of or in addition to an anti-human A-beta protein antibody according to the current invention.

### Articles of Manufacture

In another aspect according to the current invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition that is by itself or combined with another composition effective for treating, preventing and/or diagnosing the condition and may have a sterile access port (for example, the container may be an intravenous solution bag or a vial having a stopper pierceable by a hypodermic injection needle). At least one active agent in the composition is an antibody according to the current invention. The label or package insert indicates that the composition is used for treating the condition of choice. Moreover, the article of manufacture may comprise (a) a first container with a composition contained therein, wherein the composition comprises an antibody according to the current invention; and (b) a second container with a composition contained therein, wherein the composition comprises a further cytotoxic or otherwise therapeutic agent. The article of manufacture in this embodiment may further comprise a package insert indicating that the compositions can be used to treat a particular condition. Alternatively, or additionally, the article of manufacture may further comprise a second (or third) container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate as reported herein in place of or in addition to an antibody according to the current invention.

The following figures, sequences and examples are provided to aid the understanding of the present invention, the true scope of which is set forth in the appended claims.

It is understood that modifications can be made in the procedures set forth without departing from the spirit of the invention.

### DESCRIPTION OF THE FIGURES

FIG. 1 HIC chromatogram of gantenerumab.

FIG. 2 HIC chromatogram of mAb 663.

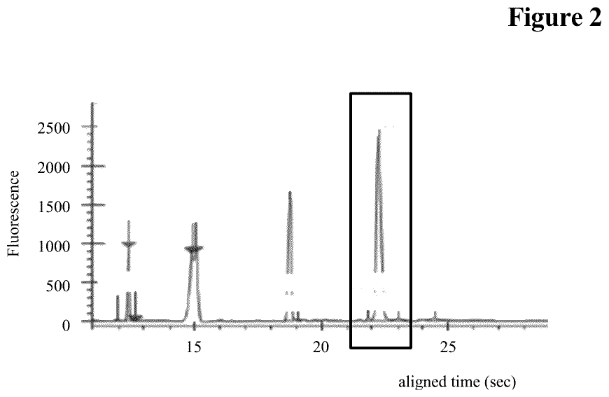

FIG. 3 HIC chromatogram of mAb 675.

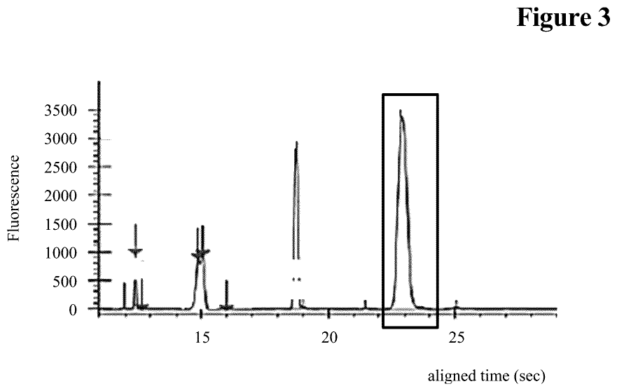

FIG. 4 In vitro A-beta plaque decoration by gantenerumab.

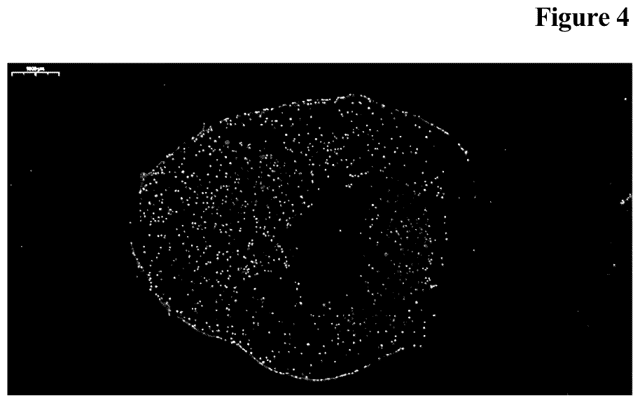

FIG. 5 In vitro A-beta plaque decoration by mAb 675.

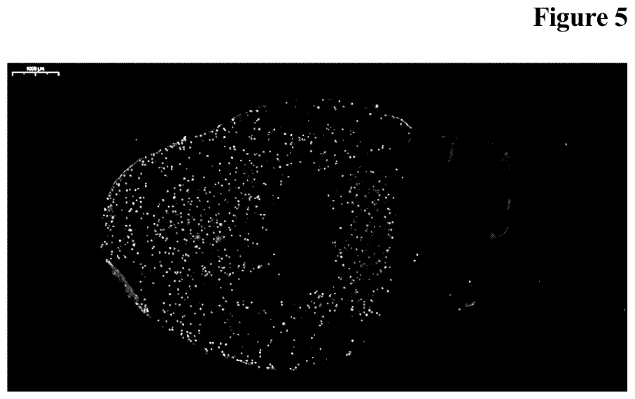

FIG. 6 In vitro A-beta plaque decoration by mAb 663.

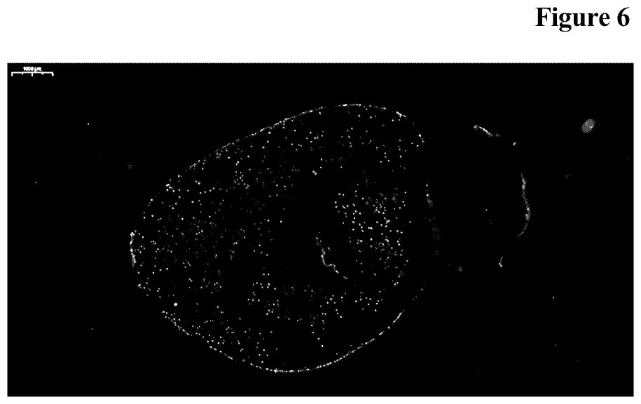

FIG. 7 In vitro A-beta plaque decoration by mAb 651.

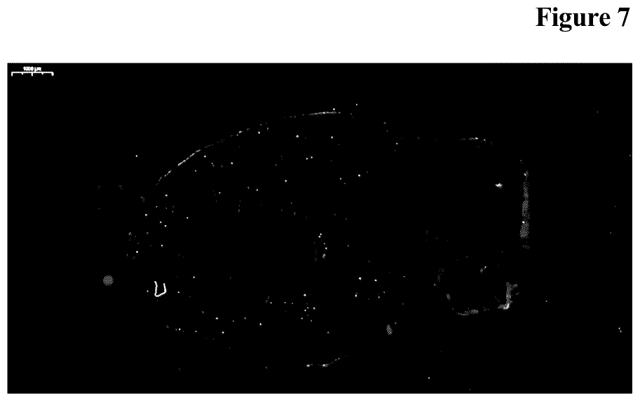

FIG. 8 In vivo A-beta plaque decoration results.

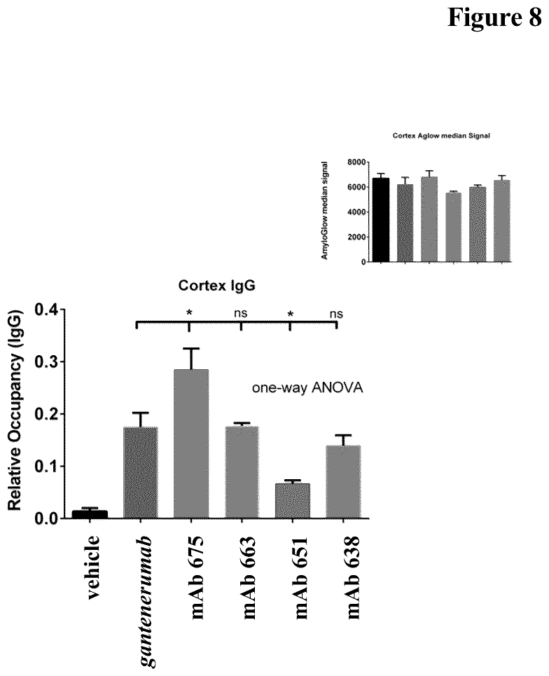

FIG. 9 In vivo A-beta plaque decoration results.

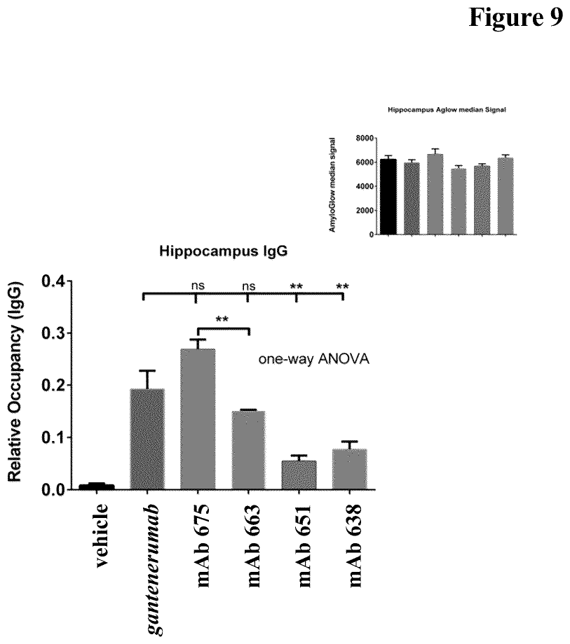

FIG. 10 Pharmacokinetic results.

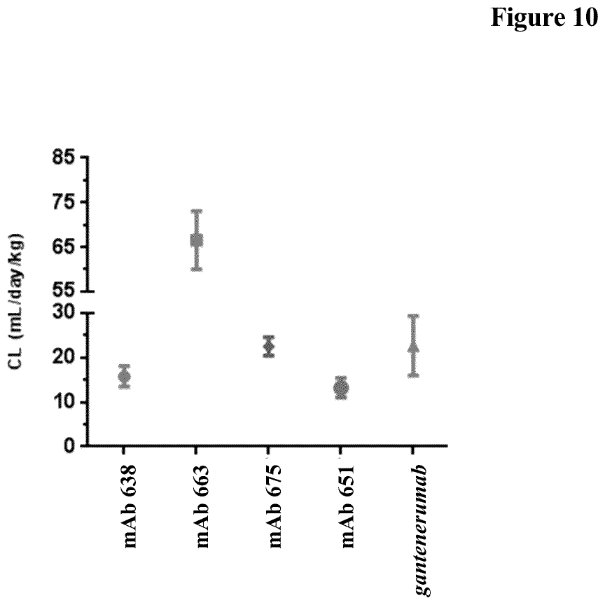

### Description of the Sequences

SEQ ID NO—description

| | |
|---|---|
| 1 | Human IgG1 heavy chain constant region polypeptide |
| 2 | Human IgG2 heavy chain constant region polypeptide |
| 3 | Human IgG3 heavy chain constant region polypeptide |
| 4 | Human IgG4 heavy chain constant region |
| 5 | Human IgG1 Fc-region polypeptide |
| 6 | Human IgG1 Fc-region derived Fc-region polypeptide with the |
| | mutations L234A, L235A |
| 7 | Human IgG1 Fc-region derived Fc-region polypeptide with Y349C, |
| | T366S, L368A and Y407V mutations |
| 8 | Human IgG1 Fc-region derived Fc-region polypeptide with S354C, |
| | T366W mutations |
| 9 | Human IgG1 Fc-region derived Fc-region polypeptide with L234A, |
| | L235A mutations and Y349C, T366S, L368A, Y407V mutations |
| 10 | Human IgG1 Fc-region derived Fc-region polypeptide with a |
| | L234A, L235A and S354C, T366W mutations |
| 11 | Human IgG1 Fc-region derived Fc-region polypeptide with a |
| | P329G mutation |
| 12 | Human IgG1 Fc-region derived Fc-region polypeptide with L234A, |
| | L235A mutations and P329G mutation |
| 13 | Human IgG1 Fc-region derived Fc-region polypeptide with a |
| | P329G mutation and Y349C, T366S, L368A, Y407V mutations |
| 14 | Human IgG1 Fc-region derived Fc-region polypeptide with a |
| | P329G mutation and S354C, T366W mutation |
| 15 | Human IgG1 Fc-region derived Fc-region polypeptide with L234A, |
| | L235A, P329G and Y349C, T366S, L368A, Y407V mutations |
| 16 | Human IgG1 Fc-region derived Fc-region polypeptide with L234A, |
| | L235A, P329G mutations and S354C, T366W mutations |
| 17 | Human IgG4 Fc-region polypeptide |
| 18 | Human IgG4 Fc-region derived Fc-region polypeptide with S228P |
| | and L235E mutations |
| 19 | Human IgG4 Fc-region derived Fc-region polypeptide with S228P, |
| | L235E mutations and P329G mutation |
| 20 | Human IgG4 Fc-region derived Fc-region polypeptide with S354C, |
| | T366W mutations |
| 21 | Human IgG4 Fc-region derived Fc-region polypeptide with Y349C, |
| | T366S, L368A, Y407V mutations |
| 22 | Human IgG4 Fc-region derived Fc-region polypeptide with a |
| | S228P, L235E and S354C, T366W mutations |
| 23 | Human IgG4 Fc-region derived Fc-region polypeptide with a |
| | S228P, L235E and Y349C, T366S, L368A, Y407V mutations |
| 24 | Human IgG4 Fc-region derived Fc-region polypeptide with a |
| | P329G mutation |
| 25 | Human IgG4 Fc-region derived Fc-region polypeptide with a |
| | P329G and Y349C, T366S, L368A, Y407V mutations |
| 26 | Human IgG4 Fc-region derived Fc-region polypeptide with a |
| | P329G and S354C, T366W mutations |
| 27 | Human IgG4 Fc-region derived Fc-region polypeptide with a |
| | S228P, L235E, P329G and Y349C, T366S, L368A, Y407V |
| | mutations |
| 28 | Human IgG4 Fc-region derived Fc-region polypeptide with a |
| | S228P, L235E, P329G and S354C, T366W mutations |
| 29 | Human kappa constant domain |
| 30 | Human lambda constant domain |
| 31 | peptidic linker (G4S)4 |
| 32 | peptidic linker (G4S)6GG |
| 33 | L3 |
| 34 | 2L |
| 35 | LoxFAS |
| 36 | SV40 polyA signal sequence |
| 37 | bGH polyA signal sequence |
| 38 | hGT terminator sequence |
| 39 | SV40 promoter sequence |
| 40 | puromycin AA |
| 41 | puromycin CDS |
| 42 | CMV promoter 1 |
| 43 | CMV promoter 2 |
| 44 | CMV promoter wild-type |
| 45 | Human A-beta 42 protein |
| 46 | Human transferrin receptor 1 |
| 47 | Human CD20 |
| 48 | Human alpha synuclein |
| 49 | Human TREM2 |
| 50 | anti-pTau antibody HC |
| 51 | anti-pTau antibody LC |
| 52 | anti-Her3 antibody HC |
| 53 | anti-Her3 antibody LC |
| 54 | anti-CD20 antibody VH |
| 55 | anti-CD20 antibody VL |
| 56 | humanized anti-alpha synuclein antibody 9E4 VH |
| 57 | humanized anti-alpha synuclein antibody 9E4 VL |
| 58 | humanized anti-alpha synuclein antibody 5C1 VH |
| 59 | humanized anti-alpha synuclein antibody 5C1 VL |
| 60 | humanized anti-alpha synuclein antibody 1H7 VH |
| 61 | humanized anti-alpha synuclein antibody 1H7 VL |
| 62 | humanized anti-alpha synuclein antibody 0017 VH |
| 63 | humanized anti-alpha synuclein antibody 0017 VL |
| 64 | humanized anti-alpha synuclein antibody 0018 VH |
| 65 | humanized anti-alpha synuclein antibody 0018 VL |
| 66 | humanized anti-alpha synuclein antibody 0081 VH |
| 67 | humanized anti-alpha synuclein antibody 0081 VL |
| 68 | anti-TfR antibody VH |
| 69 | anti-TfR antibody VH-CDR1 |
| 70 | anti-TfR antibody VH-CDR2 |
| 71 | anti-TfR antibody VH-CDR3 |
| 72 | anti-TfR antibody VL |
| 73 | anti-TfR antibody VL-CDR1 |
| 74 | anti-TfR antibody VL-CDR2 |
| 75 | anti-TfR antibody VL-CDR3 |
| 76 | gantenerumab VH |
| 77 | gantenerumab VH-CDR1 |
| 78 | gantenerumab VH-CDR2 |
| 79 | gantenerumab VH-CDR3 |
| 80 | gantenerumab VL |
| 81 | gantenerumab VL-CDR1 |
| 82 | gantenerumab VL-CDR2 |
| 83 | gantenerumab VL-CDR3 |
| 84 | gantenerumab VH NAT variant |
| 85 | gantenerumab VH-CDR1 NAT variant |
| 86 | gantenerumab VH-CDR2 NAT variant |
| 87 | gantenerumab VH-CDR3 NAT variant |
| 88 | gantenerumab VH QAS variant |
| 89 | gantenerumab VH CDR-2 QAS variant |
| 90 | gantenerumab VL variant |
| 91 | gantenerumab VL CDR-3 variant |
| 92 | anti-TREM2 antibody mAb21 VH |
| 93 | anti-TREM2 antibody mAb21 VL |
| 94 | anti-TREM2 antibody mAb52 VH |
| 95 | anti-TREM2 antibody mAb52 VL |
| 96 | anti-TREM2 antibody 7E8 VH |
| 97 | anti-TREM2 antibody 7E8 VL |
| 98 | anti-TREM2 antibody 8F8 VH |
| 99 | anti-TREM2 antibody 8F8 VL |
| 100 | anti-TREM2 antibody 9F5 VH |
| 101 | anti-TREM2 antibody 9F5 VL |
| 102 | anti-TREM2 antibody Pi 1.02.002 VH |
| 103 | anti-TREM2 antibody Pi 1.02.002 VL |
| 104 | anti-TREM2 antibody Pi 1.02.007 VH |
| 105 | anti-TREM2 antibody Pi1.02.007 VL |
| 106 | anti-TREM2 antibody AL2p58 VH |
| 107 | anti-TREM2 antibody AL2p58 VL |
| 108 | anti-TREM2 antibody RS9.F6 VH |
| 109 | anti-TREM2 antibody RS9.F6 VL |
| 110 | anti-TREM2 antibody 6E7 VH |
| 111 | anti-TREM2 antibody 6E7 VL |
| 112 | anti-TREM2 antibody antibody 1 VH |
| 113 | anti-TREM2 antibody antibody 1 VL |
| 114 | anti-TREM2 antibody GXC101 VH |
| 115 | anti-TREM2 antibody GXC101 VL |
| 116 | anti-TREM2 antibody HJ23.4 VH |
| 117 | anti-TREM2 antibody HJ23.4 VL |
| 118 | anti-TREM2 antibody MOR42596 VH |
| 119 | anti-TREM2 antibody MOR42596 VL |

### EXAMPLES

### Example 1

### Materials & General Methods

General information regarding the nucleotide sequences of human immunoglobulins light and heavy chains is given in: Kabat, E. A., et al., Sequences of Proteins of Immunological Interest, 5th ed., Public Health Service, National Institutes of Health, Bethesda, MD (1991). Amino acids of antibody chains are numbered and referred to according to numbering according to Kabat (Kabat, E. A., et al., Sequences of Proteins of Immunological Interest, 5th ed., Public Health Service, National Institutes of Health, Bethesda, MD (1991)).

### Recombinant DNA Techniques

Standard methods were used to manipulate DNA as described in Sambrook, J. et al., Molecular Cloning: A laboratory manual; Cold Spring Harbor Laboratory Press, Cold Spring Harbor, New York, 1989. The molecular biological reagents were used according to the manufacturer's instructions.

### Gene Synthesis

Desired gene segments were prepared from oligonucleotides made by chemical synthesis. The long gene segments, which were flanked by singular restriction endonuclease cleavage sites, were assembled by annealing and ligating oligonucleotides including PCR amplification and subsequently cloned via the indicated restriction sites. The DNA sequences of the subcloned gene fragments were confirmed by DNA sequencing. Gene synthesis fragments were ordered according to given specifications at Geneart (Regensburg, Germany).

### DNA Sequence Determination

DNA sequences were determined by double strand sequencing performed at MediGenomix GmbH (Martinsried, Germany) or SequiServe GmbH (Vaterstetten, Germany).

### DNA and Protein Sequence Analysis and Sequence Data Management

The GCG's (Genetics Computer Group, Madison, Wisconsin) software package version 10.2 and Infomax's Vector NT1 Advance suite version 8.0 was used for sequence creation, mapping, analysis, annotation and illustration.

### Expression Vectors

For the expression of the antibodies according to the current invention, expression plasmids for transient expression (e.g. in HEK293 cells) based either on a cDNA organization with or without a CMV-intron A promoter or on a genomic organization with a CMV promoter can be applied.

Beside the antibody expression cassette, the vectors contain:
an origin of replication which allows replication of this plasmid in E. coli, and
a β-lactamase gene which confers ampicillin resistance in E. coli.

The transcription unit of the antibody gene is composed of the following elements:
unique restriction site(s) at the 5′ end
the immediate early enhancer and promoter from the human cytomegalovirus,
the intron A sequence in the case of cDNA organization,
a 5′-untranslated region derived from a human antibody gene,
an immunoglobulin heavy chain signal sequence,
the respective antibody chain encoding nucleic acid either as cDNA or with genomic exon-intron organization,
a 3′ untranslated region with a polyadenylation signal sequence, and
unique restriction site(s) at the 3′ end.

The fusion genes encoding the antibody chains are generated by PCR and/or gene synthesis and assembled by known recombinant methods and techniques by connection of the according nucleic acid segments e.g. using unique restriction sites in the respective vectors. The subcloned nucleic acid sequences are verified by DNA sequencing. For transient transfections, larger quantities of the plasmids are prepared by plasmid preparation from transformed E. coli cultures (Nucleobond AX, Macherey-Nagel).

### Cell Culture Techniques

Standard cell culture techniques as described in Current Protocols in Cell Biology (2000), Bonifacino, J. S., Dasso, M., Harford, J. B., Lippincott-Schwartz, J. and Yamada, K. M. (eds.), John Wiley & Sons, Inc., are used.

Transient transfections in HEK293-F system

The antibodies were produced by transient expression. Therefore a transfection with the respective plasmids using the HEK293-F system (Invitrogen) according to the manufacturer's instruction was done. Briefly, HEK293-F cells (Invitrogen) growing in suspension either in a shake flask or in a stirred fermenter in serum-free FreeStyle™ 293 expression medium (Invitrogen) were transfected with a mix of the respective expression plasmids and 293fectin™ or fectin (Invitrogen). For 2 L shake flask (Corning) HEK293-F cells are seeded at a density of 1.0*1E6 cells/mL in 600 mL and incubated at 120 rpm, 8% CO2. On the next day the cells are transfected at a cell density of approx. 1.5*1E6 cells/mL with approx. 42 mL of a mixture of A) 20 mL Opti-MEM medium (Invitrogen) comprising 600 μg total plasmid DNA (1 μg/mL) and B) 20 ml Opti-MEM medium supplemented with 1.2 mL 293 fectin or fectin (2 μl/mL). According to the glucose consumption, glucose solution is added during the course of the fermentation. The supernatant containing the secreted antibody is harvested after 5-10 days and antibodies are either directly purified from the supernatant or the supernatant is frozen and stored.

### Protein Determination

The protein concentration of purified antibodies and derivatives was determined by determining the optical density (OD) at 280 nm, using the molar extinction coefficient calculated on the basis of the amino acid sequence according to Pace, et al., Protein Science 4 (1995) 2411-1423.

Antibody concentration determination in supernatants

The concentration of antibodies in cell culture supernatants was estimated by immunoprecipitation with protein A agarose-beads (Roche Diagnostics GmbH, Mannheim, Germany). Therefore, 60 μL protein A Agarose beads were washed three times in TBS-NP40 (50 mM Tris buffer, pH 7.5, supplemented with 150 mM NaCl and 1% Nonidet-P40). Subsequently, 1-15 mL cell culture supernatant was applied to the protein A Agarose beads pre-equilibrated in TBS-NP40. After incubation for at 1 hour at room temperature the beads were washed on an Ultrafree-MC-filter column (Amicon) once with 0.5 mL TBS-NP40, twice with 0.5 mL 2× phosphate buffered saline (2×PBS, Roche Diagnostics GmbH, Mannheim, Germany) and briefly four times with 0.5 mL 100 mM Na-citrate buffer (pH 5.0). Bound antibody was eluted by addition of 35 μl NuPAGE® LDS sample buffer (Invitrogen). Half of the sample was combined with NuPAGE® sample reducing agent or left unreduced, respectively, and heated for 10 min at 70° C. Consequently, 5-30 μl were applied to a 4-12% NuPAGE® Bis-Tris SDS-PAGE gel (Invitrogen) (with MOPS buffer for non-reduced SDS-PAGE and MES buffer with NuPAGE® antioxidant running buffer additive (Invitrogen) for reduced SDS-PAGE) and stained with Coomassie Blue.

The concentration of the antibodies in cell culture supernatants was quantitatively measured by affinity HPLC chromatography. Briefly, cell culture supernatants containing antibodies that bind to protein A were applied to an Applied Biosystems Poros A/20 column in 200 mM KH₂PO₄, 100 mM sodium citrate, pH 7.4 and eluted with 200 mM NaCl, 100 mM citric acid, pH 2.5 on an Agilent HPLC 1100 system. The eluted antibody was quantified by UV absorbance and integration of peak areas. A purified standard IgG1 antibody served as a standard.

Alternatively, the concentration of antibodies and derivatives in cell culture supernatants was measured by Sandwich-IgG-ELISA. Briefly, StreptaWell High Bind Streptavidin A-96 well microtiter plates (Roche Diagnostics GmbH, Mannheim, Germany) were coated with 100 μL/well biotinylated anti-human IgG capture molecule F(ab′)2<h-Fcγ>BI (Dianova) at 0.1 μg/mL for 1 hour at room temperature or alternatively overnight at 4° C. and subsequently washed three times with 200 μL/well PBS, 0.05% Tween (PBST, Sigma). Thereafter, 100 μL/well of a dilution series in PBS (Sigma) of the respective antibody containing cell culture supernatants was added to the wells and incubated for 1-2 hour on a shaker at room temperature. The wells were washed three times with 200 μL/well PBST and bound antibody was detected with 100 μl F(ab′)2<hFcγ>POD (Dianova) at 0.1 μg/mL as the detection antibody by incubation for 1-2 hours on a shaker at room temperature. Unbound detection antibody was removed by washing three times with 200 μL/well PBST. The bound detection antibody was detected by addition of 100 μL ABTS/well followed by incubation. Determination of absorbance was performed on a Tecan Fluor Spectrometer at a measurement wavelength of 405 nm (reference wavelength 492 nm).

### Preparative Antibody Purification

Antibodies were purified from filtered cell culture supernatants referring to standard protocols. In brief, antibodies were applied to a protein A Sepharose column (GE healthcare) and washed with PBS. Elution of antibodies was achieved at pH 2.8 followed by immediate neutralization. Aggregated protein was separated from monomeric antibodies by size exclusion chromatography (Superdex 200, GE Healthcare) in PBS or in 20 mM Histidine buffer comprising 150 mM NaCl (pH 6.0). Monomeric antibody fractions were pooled, concentrated (if required) using e.g., a MILLIPORE Amicon Ultra (30 MWCO) centrifugal concentrator, frozen and stored at −20° C. or −80° C. Part of the samples were provided for subsequent protein analytics and analytical characterization e.g. by SDS-PAGE, size exclusion chromatography (SEC) or mass spectrometry.

### SDS-PAGE

The NuPAGE® Pre-Cast gel system (Invitrogen) was used according to the manufacturer's instructions. In particular, 10% or 4-12% NuPAGE® Novex® Bis-TRIS Pre-Cast gels (pH 6.4) and a NuPAGE® MES (reduced gels, with NuPAGE® antioxidant running buffer additive) or MOPS (non-reduced gels) running buffer was used.

### CE-SDS

Purity and antibody integrity were analyzed by CE-SDS using microfluidic Labchip technology (PerkinElmer, USA). Therefore, 5 μl of antibody solution was prepared for CE-SDS analysis using the HT Protein Express Reagent Kit according to the manufacturer's instructions and analyzed on LabChip GXII system using a HT Protein Express Chip. Data were analyzed using LabChip GX Software.

### Analytical Size Exclusion Chromatography

Size exclusion chromatography (SEC) for the determination of the aggregation and oligomeric state of antibodies was performed by HPLC chromatography. Briefly, protein A purified antibodies were applied to a Tosoh TSKgel G3000SW column in 300 mM NaCl, 50 mM KH₂PO₄/K2HPO₄ buffer (pH 7.5) on an Dionex Ultimate® system (Thermo Fischer Scientific), or to a Superdex 200 column (GE Healthcare) in 2×PBS on a Dionex HPLC-System. The eluted antibody was quantified by UV absorbance and integration of peak areas. BioRad Gel Filtration Standard 151-1901 served as a standard.

### Mass Spectrometry

This section describes the characterization of the bispecific antibodies with emphasis on their correct assembly. The expected primary structures were analyzed by electrospray ionization mass spectrometry (ESI-MS) of the deglycosylated intact antibody and in special cases of the deglycosylated/limited LysC digested antibody.

The antibodies were deglycosylated with N-Glycosidase F in a phosphate or Tris buffer at 37° C. for up to 17 h at a protein concentration of 1 mg/ml. The limited LysC (Roche Diagnostics GmbH, Mannheim, Germany) digestions were performed with 100 μg deglycosylated antibody in a Tris buffer (pH 8) at room temperature for 120 hours, or at 37° C. for 40 min, respectively. Prior to mass spectrometry the samples were desalted via HPLC on a Sephadex G25 column (GE Healthcare). The total mass was determined via ESI-MS on a maXis 4G UHR-QTOF MS system (Bruker Daltonik) equipped with a TriVersa NanoMate source (Advion).

### Chemical Degradation Test

Samples were split into three aliquots and re-buffered into 20 mM His/His*HCl, 140 mM NaCl, pH 6.0 or into PBS, respectively, and stored at 40° C. (His/NaCl) or 37° C. (PBS). A control sample was stored at −80° C.

After incubation ended, samples were analyzed for relative active concentration (BIAcore), aggregation (SEC) and fragmentation (capillary electrophoresis or SDS-PAGE) and compared with the untreated control.

### Thermal Stability

Samples were prepared at a concentration of 1 mg/mL in 20 mM Histidine/Histidine chloride, 140 mM NaCl, pH 6.0, transferred into an optical 384-well plate by centrifugation through a 0.4 μm filter plate and covered with paraffin oil. The hydrodynamic radius was measured repeatedly by dynamic light scattering on a DynaPro Plate Reader (Wyatt) while the samples were heated with a rate of 0.05° C./min from 25° C. to 80° C.

Alternatively, samples were transferred into a 10 μL micro-cuvette array and static light scattering data as well as fluorescence data upon excitation with a 266 nm laser were recorded with an Optim1000 instrument (Avacta Inc.), while they were heated at a rate of 0.1° C./min from 25° C. to 90° C.

The aggregation onset temperature is defined as the temperature at which the hydrodynamic radius (DLS) or the scattered light intensity (Optim1000) starts to increase.

Alternatively, samples were transferred in a 9 μL multi-cuvette array. The multi-cuvette array was heated from 35° C. to 90° C. at a constant rate of 0.1° C./minute in an Optim1000 instrument (Avacta Analytical Inc.). The instrument continuously records the intensity of scattered light of a 266 nm laser with a data point approximately every 0.5° C. Light scattering intensities were plotted against the temperature. The aggregation onset temperature (T_{agg}) is defined as the temperature at which the scattered light intensity begins to increase.

The melting temperature is defined as the inflection point in fluorescence intensity vs. wavelength graph.

### Mice

B6.Cg-Fcgrt^{tmlDcr} Tg(FCGRT)276Dcr mice deficient in mouse FcRn-chain gene, but hemizygous transgenic for a human FcRn-chain gene (muFcRn−/− huFcRn tg +/−, line 276) were used for the pharmacokinetic studies. Mouse husbandry was carried out under specific pathogen free conditions. Mice were obtained from the Jackson Laboratory (Bar Harbor, ME, USA) (female, age 4-10 weeks, weight 17-22 g at time of dosing). All animal experiments were approved by the Government of Upper Bavaria, Germany (permit number 55.2-1-54-2532.2-28-10) and performed in an AAALAC accredited animal facility according to the European Union Normative for Care and Use of Experimental Animals. The animals were housed in standard cages and had free access to food and water during the whole study period.

### Pharmacokinetic Studies

A single dose of antibody was injected i.v. via the lateral tail vein at a dose level of 10 mg/kg. The mice were divided into 3 groups of 6 mice each to cover 9 serum collection time points in total (at 0.08, 2, 8, 24, 48, 168, 336, 504 and 672 hours post dose). Each mouse was subjected twice to retro-orbital bleeding, performed under light anesthesia with Isoflurane™ (CP-Pharma GmbH, Burgdorf, Germany); a third blood sample was collected at the time of euthanasia. Blood was collected into serum tubes (Microvette 500Z-Gel, Sarstedt, Numbrecht, Germany). After 2 h incubation, samples were centrifuged for 3 min at 9.300 g to obtain serum. After centrifugation, serum samples were stored frozen at −20° C. until analysis.

PK analysis

The pharmacokinetic parameters were calculated by non-compartmental analysis using WinNonlin™ 1.1.1 (Pharsight, CA, USA).

Briefly, area under the curve (AUC_{0-inf}) values were calculated by logarithmic trapezoidal method due to non-linear decrease of the antibodies and extrapolated to infinity using the apparent terminal rate constant λz, with extrapolation from the observed concentration at the last time point.

Plasma clearance was calculated as Dose rate (D) divided by AUC_{0-inf}. The apparent terminal half-life (T1/2) was derived from the equation T1/2=In2/λz.

### Example 2

### Expression and Purification

The antibodies were produced as described above in the general materials and methods section.

The antibodies were purified from the supernatant by a combination of protein A affinity chromatography and size exclusion chromatography. The obtained products were characterized for identity by mass spectrometry and analytical properties such as purity by CE-SDS, monomer content and stability.

The expected primary structures were analyzed by electrospray ionization mass spectrometry (ESI-MS) of the deglycosylated intact antibody and deglycosylated/plasmin digested or alternatively deglycosylated/limited LysC digested antibody as described in the general methods section.

Additional analytical methods (e.g. thermal stability, mass spectrometry and functional assessment) were only applied after protein A and SEC purification.

### Example 3

Determination of binding to Aβ1-40 fibers in vitro by ELISA

Binding of the antibodies to fibrillar Aβ is measured by an ELISA assay. Briefly, AD(1-40) is coated at 7 μg/mL in PBS onto Maxisorb plates for 3 days at 37° C. to produce fibrillar Abeta, and then dried for 3 h at RT. The plate is blocked with 1% CroteinC and 0.1% RSA in PBS (blocking buffer) for 1 h at RT, then washed once with wash buffer. Antibodies or controls are added at concentrations up to 100 nM in blocking buffer and incubated at 4° C. overnight. After 4 wash steps, constructs are detected by addition of anti-human-IgG-HRP (Jackson Immunoresearch) at 1:10,000 dilution in blocking buffer (1 RT), followed by 6 washes and incubation in TMB (Sigma). Absorbance is read out at 450 nm after stopping color development with 1 N HCl.

### Example 4

Staining of Native Human β-Amyloid Plaques from Brain Sections of an Alzheimer's Disease Patient by Indirect Immunofluorescence Using an Antibody According to the Current Invention

The antibodies can be tested for the ability to stain native human R amyloid plaques by immunohistochemistry analysis using indirect immunofluorescence. Specific and sensitive staining of genuine human β-amyloid plaques can be demonstrated. Cryostat sections of unfixed tissue from the temporal cortex obtained postmortem from patients positively diagnosed for Alzheimer's disease are labeled by indirect immunofluorescence. A two-step incubation is used to detect bound bispecific antibody, which is revealed by affinity-purified goat anti-human (GAH555) IgG (H+L) conjugated to Alexa 555 dye (Molecular Probes). Controls can include unrelated human IgG1 antibodies (Sigma) and the secondary antibody alone, which all should give negative results.

### Example 5

### In Vivo β-Amyloid Plaque Decoration by an Antibody According to the Current Invention in a Mouse Model of Alzheimer's Disease

Antibodies were tested in APP/PS2 double transgenic mice, a mouse model for AD-related amyloidosis (Richards, J. Neuroscience, 23 (2003) 8989-9003) for their ability to immuno-decorate 0-amyloid plaques in vivo. This enabled assessment of the extent of brain penetration and binding to amyloid-D plaques. The antibodies were administered at different doses and after 6 days, animals are perfused with phosphate-buffered saline and the brains frozen on dry ice and prepared for cryosectioning.

The presence of the antibodies bound to D-amyloid plaques were assessed using unfixed cryostat sections either by single-labeled indirect immunofluorescence with goat anti-human IgG (H+L) conjugated to Alexa555 dye (GAH555) (Molecular Probes) at a concentration of 15 μg/ml for 1 hour at room temperature. A counterstaining for amyloid plaques can be done by incubation with BAP-2, a mouse monoclonal antibody against AR conjugated to Alexa 488 at a concentration of 0.5 μg/ml for 1 hour at room temperature. Slides are embedded with fluorescence mounting medium (53023 Dako) and imaging is done by confocal laser microscopy.

### Example 6

### FcRn and Heparin Affinity Chromatography

### Preparation of FcRn Affinity Column

### Expression of FcRn in HEK293 Cells

FcRn was transiently expressed by transfection of HEK293 cells with two plasmids containing the coding sequence of FcRn and of beta-2-microglobulin. The transfected cells were cultured in shaker flasks at 36.5° C., 120 rpm (shaker amplitude 5 cm), 80% humidity and 7% CO2. The cells were diluted every 2-3 days to a density of 3 to 4*105 cells/ml.

For transient expression, a 14 l stainless steel bioreactor was started with a culture volume of 8 l at 36.5° C., pH 7.0±0.2, pO₂ 35% (gassing with N2 and air, total gas flow 200 ml min-1) and a stirrer speed of 100-400 rpm. When the cell density reached 20*105 cells/ml, 10 mg plasmid DNA (equimolar amounts of both plasmids) was diluted in 400 ml Opti-MEM (Invitrogen). 20 ml of 293fectin (Invitrogen) was added to this mixture, which was then incubated for 15 minutes at room temperature and subsequently transferred into the fermenter. From the next day on, the cells were supplied with nutrients in continuous mode: a feed solution was added at a rate of 500 ml per day and glucose as needed to keep the level above 2 g/l. The supernatant was harvested 7 days after transfection using a swing head centrifuge with 1 l buckets: 4000 rpm for 90 minutes. The supernatant (13 L) was cleared by a Sartobran P filter (0.45 μm+0.2 μm, Sartorius) and the FcRn beta-2-microglobulin complex was purified therefrom.

### Biotinylation of Neonatal Fc Receptor

3 mg FcRn beta-2-microglobulin complex were solved/diluted in 5.3 mL 20 mM sodium dihydrogen phosphate buffer containing 150 mM sodium chloride and added to 250 μL PBS and 1 tablet complete protease inhibitor (complete ULTRA Tablets, Roche Diagnostics GmbH). FcRn was biotinylated using the biotinylation kit from Avidity according to the manufacturer instructions (Bulk BIRA, Avidity LLC). The biotinylation reaction was done at room temperature overnight.

The biotinylated FcRn was dialyzed against 20 mM MES buffer comprising 140 mM NaCl, pH 5.5 (buffer A) at 4° C. overnight to remove excess of biotin.

### Coupling to Streptavidin Sepharose

For coupling to streptavidin Sepharose, 1 mL streptavidin Sepharose (GE Healthcare, United Kingdom) was added to the biotinylated and dialyzed FcRn beta-2-microglobulin complex and incubated at 4° C. overnight. The FcRn beta-2-microglobulin complex derivatized Sepharose was filled in a 4.6 mm×50 mm chromatographic column (Repligen). The column was stored in 80% buffer A and 20% buffer B (20 mM Tris(hydroxymethyl)aminomethane pH 8.8, 140 mM NaCl).

Chromatography using FcRn affinity column and pH gradient

### Conditions:

column dimensions: 50 mm×4.6 mm
loading: 30 μg sample
buffer A: 20 mM MES, with 140 mM NaCl, adjusted to pH 5.5
buffer B: 20 mM Tris/HCl, with 140 mM NaCl, adjusted to pH 8.8

30 μg of samples were applied onto the FcRn affinity column equilibrated with buffer A. After a washing step of 10 minutes in 20% buffer B at a flow rate of 0.5 mL/min, elution was performed with a linear gradient from 20% to 70% buffer B over 70 minutes. The UV light absorption at a wavelength of 280 nm was used for detection. The column was regenerated for 10 minutes using 20% buffer B after each run.

For the calculation of relative retention times, a standard sample (anti-Her3 antibody (SEQ ID NO: 52 and 53), oxidized for 18 hours with 0.02% hydrogen peroxide according to (Bertoletti-Ciarlet, A., et al., Mol. Immunol. 46 (2009) 1878-1882) was run at the beginning of a sequence and after each 10 sample injections.

Briefly, the antibody (at 9 mg/mL) in 10 mM sodium phosphate pH 7.0 was mixed with H₂O₂ to a final concentration of 0.02% and incubated at room temperature for 18 h. To quench the reaction the samples were thoroughly dialyzed into pre-cooled 10 mM sodium acetate buffer pH 5.0.

Relative retention times were calculated according to the following equation:

$$t\_ ( rel , i ) = ( t\_i - t\_peak 2 ) / ( t\_peak 3 - t\_peak 2 )$$

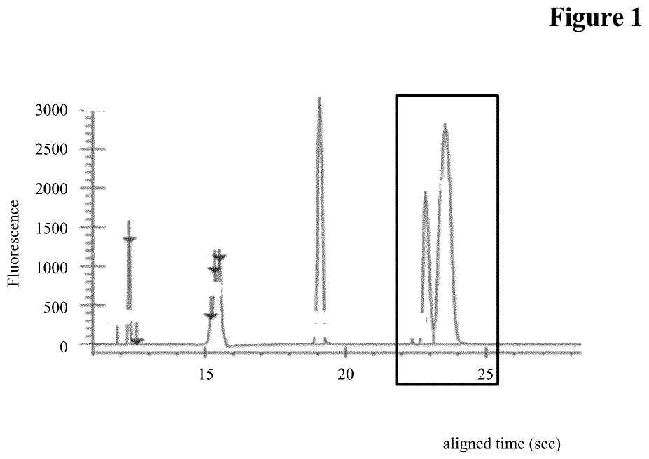

For peak definition, see FIG. 1 of WO 2018/197533.

### Chromatography Using Heparin Affinity Column and pH Gradient

### Conditions:

column dimensions: 50 mm×5.0 mm
loading: 20-50 μg sample
buffer A: 50 mM TRIS pH 7.4
buffer B: 50 mM TRIS pH 7.4, 1000 mM NaCl

20 to 50 μg of protein samples in low-salt buffer (<25 mM ionic strength) were applied to a TSKgel Heparin-5PW Glass column, 5.0×50 mm (Tosoh Bioscience, Tokyo/Japan), which was pre-equilibrated with buffer A at room temperature. Elution was performed with a linear gradient from 0-100% buffer B over 32 minutes at a flow rate of 0.8 mg/mL. The UV light absorption at a wavelength of 280 nm was used for detection. Every injection sequence started with a retention time standard (anti-pTau antibody; SEQ ID NO: 50 and 51) which was used to calculate relative retention times according to the following formula:

$$t\_ ( rel , i ) = t\_i / t\_pTau$$

(trel,i: relative retention time of peak i; ti: retention time of peak i; tpTau: retention time of the anti-pTau antibody peak).

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. An antibody that binds to human A-beta protein, wherein the antibody comprises a heavy chain variable domain (VH) and a light chain variable domain comprising CDRs selected from
(1)
(a) a CDR-H1 comprising the amino acid sequence of SEQ ID NO: 85;
(b) a CDR-H2 comprising the amino acid sequence of SEQ ID NO: 86;
(c) a CDR-H3 comprising the amino acid sequence of SEQ ID NO: 87;
(d) a CDR-L1 comprising the amino acid sequence of SEQ ID NO: 81;
(e) a CDR-L2 comprising the amino acid sequence of SEQ ID NO: 82; and
(f) a CDR-L3 comprising the amino acid sequence of SEQ ID NO: 83;
or
(2)
(a) a CDR-H1 comprising the amino acid sequence of SEQ ID NO: 85;
(b) a CDR-H2 comprising the amino acid sequence of SEQ ID NO: 89;
(c) a CDR-H3 comprising the amino acid sequence of SEQ ID NO: 87;
(d) a CDR-L1 comprising the amino acid sequence of SEQ ID NO: 81;
(e) a CDR-L2 comprising the amino acid sequence of SEQ ID NO: 82; and
(f) a CDR-L3 comprising the amino acid sequence of SEQ ID NO: 83;
or
(3)
(a) a CDR-H1 comprising the amino acid sequence of SEQ ID NO: 85;
(b) a CDR-H2 comprising the amino acid sequence of SEQ ID NO: 86;
(c) a CDR-H3 comprising the amino acid sequence of SEQ ID NO: 87;
(d) a CDR-L1 comprising the amino acid sequence of SEQ ID NO: 81;
(e) a CDR-L2 comprising the amino acid sequence of SEQ ID NO: 82; and
(f) a CDR-L3 comprising the amino acid sequence of SEQ ID NO: 91;
or
(4)
(a) a CDR-H1 comprising the amino acid sequence of SEQ ID NO: 85;
(b) a CDR-H2 comprising the amino acid sequence of SEQ ID NO: 89;
(c) a CDR-H3 comprising the amino acid sequence of SEQ ID NO: 87;
(d) a CDR-L1 comprising the amino acid sequence of SEQ ID NO: 81;
(e) a CDR-L2 comprising the amino acid sequence of SEQ ID NO: 82; and
(f) a CDR-L3 comprising the amino acid sequence of SEQ ID NO: 91.

2. The antibody according to claim 1, wherein the antibody has one or more of the following properties
specifically binds to a human A-beta protein of SEQ ID NO: 45;
has a glycosylation site in the heavy chain CDR2 that has a glyco-occupancy of at least 95% as determined by CE-SDS;
is free of species with any non-glycosylated glycosylation site;
has an EC50 value for the human A-beta protein of SEQ ID NO: 45 of 0.5 nM or less;
is binding to A-beta plaques in vitro and not binding to non-A-beta protein molecules in brain samples at a staining concentration of up to and including 1 μg/mL;
is binding to A-beta plaques in vivo more strongly than gantenerumab;
has a relative occupancy in vivo of plaques in the cortex and hippocampus of more than 0.2 determine in a one-way Anova;
has a thermal stability (DLS T_{agg}/DLS Tₘ) of more than 68° C.

3. The antibody according to claim 1, wherein the antibody is a monoclonal antibody.

4. The antibody according to claim 1, wherein the antibody is an antibody fragment that binds to human A-beta protein.

5. The antibody according to claim 1, wherein the antibody comprises
(a) a VH sequence having at least 95% sequence identity to the amino acid sequence of SEQ ID NO: 84;
(b) a VL sequence having at least 95% sequence identity to the amino acid sequence of SEQ ID NO: 80; or
(c) a VH sequence as defined in (a) and a VL sequence as defined in (b).

6. The antibody according to claim 1,
wherein the antibody comprises a VH sequence of SEQ ID NO: 84 and a VL sequence of SEQ ID NO: 80.

7. The antibody according to claim 1, wherein the antibody is
a) a full-length antibody of the human subclass IgG1,
b) a full-length antibody of the human subclass IgG4,
c) a full-length antibody of the human subclass IgG1 with the mutations L234A, L235A and P329G,
d) a full-length antibody of the human subclass IgG1 with the mutations L234A, L235A and P329G in both heavy chains and the mutations T366W and S354C in one heavy chain and the mutations T366S, L368A, Y407V and Y349C in the respective other heavy chain,
e) a full-length antibody of the human subclass IgG1 with the mutations L234A, L235A and P329G in both heavy chains and the mutations T366W and Y349C in one heavy chain and the mutations T366S, L368A, Y407V and S354C in the respective other heavy chain,
f) a full-length antibody of the human subclass IgG4 with the mutations T366W and S354C in one heavy chain and the mutations T366S, L368A, Y407V and Y349C in the respective other heavy chain,
g) a full-length antibody of the human subclass IgG4 with the mutations T366W and Y349C in one heavy chain and the mutations T366S, L368A, Y407V and S354C in the respective other heavy chain,
h) a full-length antibody of the human subclass IgG1 with the mutations L234A, L235A, P329G, I253A, H310A and H435A in both heavy chains and the mutations T366W and S354C in one heavy chain and the mutations T366S, L368A, Y407V and Y349C in the respective other heavy chain,
i) a full-length antibody of the human subclass IgG1 with the mutations L234A, L235A, P329G, I253A, H310A and H435A in both heavy chains and the mutations T366W and Y349C in one heavy chain and the mutations T366S, L368A, Y407V and S354C in the respective other heavy chain,
j) a full-length antibody of the human subclass IgG1 with the mutations L234A, L235A, P329G, M252Y, S254T and T256E in both heavy chains and the mutations T366W and S354C in one heavy chain and the mutations T366S, L368A, Y407V and Y349C in the respective other heavy chain,
k) a full-length antibody of the human subclass IgG1 with the mutations L234A, L235A, P329G, M252Y, S254T and T256E in both heavy chains and the mutations T366W and Y349C in one heavy chain and the mutations T366S, L368A, Y407V and S354C in the respective other heavy chain, or
l) a full-length antibody of the human subclass IgG1 with the mutations L234A, L235A, P329G, H310A, H433A and Y436A in both heavy chains and the mutations i) T366W, and ii) S354C or Y349C, in one heavy chain and the mutations i) T366S, L368A, and Y407V, and ii) Y349C or S354C, in the respective other heavy chain, or
m) one of a) to 1) without the C-terminal lysine residue.

8. The antibody according to claim 1, wherein the antibody comprises a heavy chain comprising a heavy chain variable domain of SEQ ID NO: 84 and a heavy chain constant region of SEQ ID NO: 01 and a light chain comprising a light chain variable domain of SEQ ID NO: 80 and a light chain kappa constant domain of SEQ ID NO: 29.

9. The antibody according to claim 1, wherein the antibody comprises a heavy chain comprising a heavy chain variable domain of SEQ ID NO: 84 with a pyroglutamic acid (pE) residue instead of a glutamine (Q) residue as first N-terminal amino acid residue and a heavy chain constant region of SEQ ID NO: 01 and a light chain comprising a light chain variable domain of SEQ ID NO: 80 and a light chain kappa constant domain of SEQ ID NO: 29.

10. The antibody according to claim 1, wherein the antibody is a multispecific antibody.

11. A composition of isolated nucleic acid molecules wherein each of the isolated nucleic acid molecules of the composition encodes one chain of the antibody according to claim 1.

12. A host cell, comprising the composition of nucleic acid molecules of claim 11.

13. A method of producing an antibody that binds to human A-beta protein, comprising the steps of
culturing the host cell of claim 12 in a cultivation medium,
recovering the antibody from the host cell or/and the cultivation medium, and
purifying the antibody using one or more chromatography steps.

14. A pharmaceutical composition, comprising the antibody according to claim 1 and a pharmaceutically acceptable carrier.

15. A medicament, comprising the antibody according to claim 1 or the pharmaceutical composition according to claim 14.
